SEQUENCE LISTING (16 sequences)

SEQ ID NO: 1 - protein, 16 aa, synthetic construct
  REGION 1..16: note = T cell receptor beta chain (TRB)
CASSFSTCSANYGYTF

SEQ ID NO: 2 - protein, 17 aa, synthetic construct
  REGION 1..17: note = T cell receptor alpha chain (TRA)
CAVSDLEPNSSASKIIF

SEQ ID NO: 3 - protein, 15 aa, synthetic construct
  REGION 1..15: note = T cell receptor beta chain (TRB)
CASFSTCSANYGYTF

SEQ ID NO: 4 - RNA, 11 nt, synthetic construct
  misc_feature 1..11: note = Poly(A)
  source 1..11: mol_type = other RNA
aaaaaaaaaaa

SEQ ID NO: 5 - RNA, 15 nt, synthetic construct
  misc_feature 1..15: note = Poly(A)
  source 1..15: mol_type = other RNA
aaaaaaaaaaaaaaa

SEQ ID NO: 6 - RNA, 16 nt, synthetic construct
  misc_feature 1..16: note = Poly(A)
  source 1..16: mol_type = other RNA
aaaaaaaaaaaaaaaa

SEQ ID NO: 7 - DNA, 12 bp, synthetic construct
  misc_feature 1..12: note = Poly(C)
  source 1..12: mol_type = other DNA
cccccccccccc

SEQ ID NO: 8 - DNA, 17 bp, synthetic construct
  misc_feature 1..17: note = Poly(C)
  source 1..17: mol_type = other DNA
ccccccccccccccccc

SEQ ID NO: 9 - protein, 9 aa, synthetic construct
  REGION 1..9: note = heteroclitic peptide
ALAAAAAAV

SEQ ID NO: 10 - protein, 9 aa, synthetic construct
  REGION 1..9: note = heteroclitic peptide
ATAAAAAAK

SEQ ID NO: 11 - protein, 9 aa, synthetic construct
  REGION 1..9: note = heteroclitic peptide
AYAAAAAAL

SEQ ID NO: 12 - protein, 9 aa, synthetic construct
  REGION 1..9: note = heteroclitic peptide
APAAAAAAV

SEQ ID NO: 13 - protein, 9 aa, synthetic construct
  REGION 1..9: note = heteroclitic peptide
RYAAAAALL

SEQ ID NO: 14 - protein, 8 aa, synthetic construct
  REGION 1..8: note = heteroclitic peptide
ASYAAAAV

SEQ ID NO: 15 - protein, 8 aa, synthetic construct
  REGION 1..8: note = heteroclitic peptide
TSYKFESV

SEQ ID NO: 16 - protein, 10 aa, synthetic construct
  REGION 1..10: note = control peptide
TPGPGVRYPL

(19) United States
(12) Patent Application Publication
(10) Pub. No.: US 2026/0250663 A1
(43) Pub. Date: Aug. 27, 2026

(54) METHODS FOR CHARACTERIZATION OF ANTIGEN-BINDING MOLECULES FROM BIOLOGICAL SAMPLES

(71) Applicant: 10X Genomics, Inc., Pleasanton, CA (US)

(72) Inventors: Wyatt James McDonnell, Pleasanton, CA (US); Bruce Alexander Adams, Pleasanton, CA (US); Michael John Terry Stubbington, Bristol (GB); David Benjamin Jaffe, Pleasanton, CA (US); Christina Galonska, Stockholm (SE); Ariel Royall, Pleasanton, CA (US); Malte Kuhnemund, Stockholm (SE); Camilla Engblom, Stockholm (SE); Kim A. Thrane, Stockholm (SE); Jeffrey Eron Mold, Stockholm (SE); Jonas Frisen, Stockholm (SE); Joakim Lundeberg, Stockholm (SE); Qirong Lin, Stockholm (SE); Zachary W. Bent, Pleasanton, CA (US); Marlon Stoeckius, Stockholm (SE); Katherine Pfeiffer, Pleasanton, CA (US); Caroline Julie Gallant, Stockholm (SE)

(21) Appl. No.: 18/993,965

(22) PCT Filed: Jul. 12, 2023
(86) PCT No.: PCT/US2023/070060

§ 371 Date: Jan. 13, 2025

Related U.S. Application Data
(60) Provisional application No. 63/388,774, filed on Jul. 13, 2022.

(51) Int. Cl. C12N 15/10; C12Q 1/6804; G01N 33/53

(52) U.S. Cl. CPC C12N 15/1065; C12Q 1/6804; G01N 33/5308; G01N 2333/7051; G01N 2458/10

(57) ABSTRACT

The present disclosure relates generally to the field of immunology, and particularly relates to methods for the identification and/or characterization of target specific antigen-binding molecules (e.g., antibodies) produced by immune cells within biological samples using approaches involving barcode enabled antigen mapping.

### CROSS-REFERENCE TO RELATED APPLICATIONS

This application claims the benefit of priority to U.S. Provisional Patent Application Ser. No. 63/388,774, filed on Jul. 13, 2022. The disclosure of the above-referenced application is herein expressly incorporated by reference it its entirety, including any drawings.

### FIELD

The present disclosure relates generally to the field of immunology, and particularly relates to methods for the identification and characterization of antigen-binding molecules (e.g., antibodies) in biological samples.

### INCORPORATION OF THE SEQUENCE LISTING

This application contains a Sequence Listing, which is hereby incorporated herein by reference in its entirety. The accompanying Sequence Listing, named “057862-619001WO_SequenceListing_ST26.xml,” was created on Jul. 12, 2023 and is 21,013 bytes.

### BACKGROUND

Significant advances in analyzing and characterizing biological and biochemical materials and systems have led to unprecedented advances in understanding the mechanisms of life, health, disease and treatment. Among these advances, technologies that target and characterize the genomic make up of biological systems have yielded some of the most groundbreaking results, especially for therapeutics. In particular, a number of approaches and systems are currently available for the isolation and characterization of antigen-binding molecules (ABM, e.g., antibodies). Such ABMs that target and bind to specific antigens of interest have been developed as new immunotherapeutic agents.

Generally, however, the existing approaches and methods involve laborious processes of isolating ABMs (e.g., antibodies from activated human B-cells). Furthermore, these approaches are cumbersome, cost-prohibitive, time-consuming, not adaptable to high-throughput and inefficient at retrieving rare antibodies that are produced by a minor fraction of the total repertoire of immune cells, such as B cells. Limitations of current approaches include, e.g., (i) a lack of heavy-light chain pairing (bulk approaches), (ii) inability to efficiently amplify B cell receptor sequences due to poor RNA quality or sample preparation conditions, and (iii) generation of antibodies that are not fully humanized.

Therefore, there is a need for alternative approaches useful for the characterization of these ABMs. In particular, there is a need for improved methods and reagents that allow for reliably and rapidly identifying and characterizing ABMs capable of recognizing an antigen of interest in three-dimensional biological samples, such as tissues.

### SUMMARY

The present disclosure provides, inter alia, methods, compositions and kits for the identification and/or characterization of ABMs obtained from biological samples, using spatial immune profiling methodologies. Characterization of ABMs having desirable properties, e.g., that recognize and bind to cells or tissues displaying a particular antigen (e.g., tumor or viral antigens), can be useful in the development of new immunotherapies to treat cancers and/or infectious disease. Thus, the disclosure also provides methods to produce recombinant ABMs with desired properties (e.g., having specificity for an antigen or a biological sample, such as a tumor sample) for their subsequent characterization and use in various downstream applications, e.g., in diagnostic or therapeutic applications.

In one aspect, provided herein are methods for identifying and/or characterizing an antigen-binding molecule (ABM) in a biological sample, the methods including: a) contacting the biological sample with a plurality of antigens, wherein the plurality of antigens comprises a target antigen coupled to a first reporter oligonucleotide comprising (i) a first reporter barcode sequence that identifies the target antigen and (ii) a capture handle sequence, and wherein the contacting provides the ABM bound to the target antigen; b) providing an array of capture probes, the array comprising a first capture probe comprising a first spatial barcode and a first capture domain, and a second capture probe comprising a second spatial barcode and a second capture domain; c) hybridizing (i) the capture handle sequence of the first reporter oligonucleotide to the first capture domain of the first capture probe and (ii) a nucleic acid comprising a sequence encoding at least a portion of the ABM or a reverse complement thereof to the second capture domain of the second capture probe; d) generating a first barcoded polynucleotide comprising (i) the first reporter barcode sequence or a reverse complement thereof and (ii) the first spatial barcode or a reverse complement thereof; and e) generating a second barcoded polynucleotide comprising (i) the nucleic acid encoding at least a portion of the ABM or a reverse complement thereof and (ii) the spatial barcode or a reverse complement thereof.

In some embodiments, the methods further include f) determining the identity and spatial location of the ABM in the biological sample and/or identifying the ABM as having bound to the target antigen using the first and second barcoded polynucleotides.

The plurality of antigens can further include a non-target antigen coupled to a second reporter oligonucleotide, wherein the second reporter oligonucleotide comprises (i) a second reporter barcode sequence that identifies the non-target antigen, and (ii) the capture handle sequence. In some embodiments, the non-target antigen is an antigen to which the ABM is not expected to bind. In some embodiments, the plurality of antigens comprises two or more distinct target antigens, wherein each distinct target antigen is coupled to a reporter oligonucleotide comprising (i) a reporter barcode sequence that identifies the target antigen and (ii) the capture handle sequence.

In some embodiments, the methods further include assessing the binding specificity of the ABM. This can involve, for example: (i) identifying the ABM as having a binding specificity for the target antigen if the ABM binds to the target antigen and does not significantly bind the non-target antigen, or (ii) identifying the ABM as non-specific for the target antigen if the ABM significantly binds to the non-target antigen.

In some embodiments, the array of capture probes comprises a plurality of the first capture probe and a plurality of the second capture probe. In some embodiments, the capture handle sequence of the first reporter oligonucleotide is partially or fully complementary to the first capture domain of the first capture probe. In some embodiments, the first spatial barcode of the first capture probe is identical to the second spatial barcode of the second capture probe. In some embodiments, the first spatial barcode of the first capture probe is different from the second spatial barcode of the second capture probe.

In some embodiments, the target antigen and first reporter oligonucleotide are indirectly coupled. For example, the target antigen can be comprised in a labeling agent, wherein the labeling agent further comprises a support, and wherein (i) the target antigen is coupled to the support via a ligand, and (ii) the first reporter oligonucleotide is coupled to the support. In some embodiments, the target antigen is covalently conjugated to the ligand.

In some embodiments, the target antigen comprises a target MHC molecule complex, wherein the target MHC molecule complex comprises an MHC molecule bound to a target antigenic molecule. The target MHC molecule complex can be further coupled to a support via a ligand, and the first reporter oligonucleotide can be coupled to the support. In some embodiments, the target antigenic molecule is an antigenic peptide, a lipid, or a small molecule. In some embodiments, the target antigen comprises a plurality of target MHC molecule complexes, optionally wherein the target MHC molecule complexes are covalently conjugated to the ligand. In some embodiments, wherein the target antigen comprises a plurality of target MHC molecule complexes. For example, in some embodiments, the target antigen comprises four target MHC molecule complexes.

The support can be selected from avidin, streptavidin, deglycosylated avidin (e.g., NeutrAvidin™), traptavidin, tamavidin, xenavidin, bradavidin, AVR2 (avidin related protein 2), AVR4 (avidin related protein 4), and variants, mutants, derivatives, and homologs of any thereof. In some embodiments, the ligand comprises biotin. In some embodiments, the labeling agent or target MHC molecule complex further comprises a fluorescent agent.

In some embodiments, the ABM is expressed by a cell comprised within the biological sample. The cell can be, for example, an immune cell. Exemplary immune cells include, without limitation, B-cells and T-cells. In some embodiments, the B cell is a plasmablast, a plasma cell, a memory B cell, a regulatory B cell, or a lymphoplasmacytoid cell. In some embodiments, the ABM is an antibody or fragment thereof, or a T-cell receptor or fragment thereof. In some embodiments, the antibody is a secreted antibody. In some embodiments, the secreted antibody is in proximity to the cell. The methods can further include in step (a) contacting the biological sample with a plurality of labeling agents, wherein the labeling agents are configured to bind or otherwise couple to one or more surface features (e.g., cell surface proteins) of the cell.

In some embodiments, the biological sample is disposed (e.g., mounted) on the array. In some embodiments, the biological sample is disposed on a first substrate and the array is attached to a second substrate, and wherein the method further includes aligning the first substrate with the second substrate such that at least a portion of the biological sample is aligned with at least a portion of the array.

In some embodiments, the biological sample is disposed on a first substrate during (b) and the array is attached to a second substrate, and wherein the method includes, following (b): mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the biological sample comprising the ABM is aligned with a portion of the array of capture probes and within a threshold distance of the array of capture probes, and such that the portion of the biological sample and the capture probes contact the reagent medium. In some embodiments, the permeabilization agent releases the nucleic acid comprising a sequence encoding at least a portion of the ABM or a reverse complement thereof from the biological sample.

In some embodiments, the methods further include prior to the hybridizing in step (c), releasing the first reporter oligonucleotide and migrating the first reporter oligonucleotide to the array. In some embodiments, the nucleic acid comprising a sequence encoding at least a portion of the ABM or a reverse complement thereof is comprised in the biological sample, and the method further comprises releasing the nucleic acid comprising a sequence encoding at least a portion of the ABM or a reverse complement thereof from the biological sample, and migrating the nucleic acid comprising a sequence encoding at least a portion of the ABM or a reverse complement thereof to the array. The migrating can include passive migration or active migration. For example, active migration includes electrophoresis.

In some embodiments, the first reporter oligonucleotide and second reporter oligonucleotide further include one or more functional domains. In some embodiments, the first and/or second capture probes further include a cleavage domain, one or more functional domains, a unique molecular identifier, or a combination thereof.

In some embodiments, the first capture domain of the first capture probe and the second capture domain of the second capture probe are identical. In some embodiments, the first capture domain of the first capture probe and the second capture domain of the second capture probe are different. In some embodiments, the first capture domain of the first capture probe is a defined non-homopolymeric sequence or a homopolymeric sequence. The homopolymeric sequence can include a poly(T) sequence and/or the non-homopolymeric sequence include a fixed sequence or a degenerate sequence. In some embodiments, the second capture domain of the second capture probe hybridizes to the nucleic acid comprising a sequence encoding at least a portion of the ABM or a reverse complement thereof, wherein the ABM is an antibody or TCR. In some embodiments, the second capture domain of the second capture probe hybridizes a poly(A) sequence of the nucleic acid comprising a sequence encoding at least a portion of the antibody or TCR or a reverse complement thereof. In some embodiments, the second capture domain of the second capture probe hybridizes to the nucleic acid comprising a sequence encoding at least a portion of the ABM (e.g., antibody or TCR) or the reverse complement thereof in a region encoding a constant region of the ABM (e.g., antibody or TCR).

In some embodiments, generating a second barcoded polynucleotide in step (e) comprises extending the capture probe using the nucleic acid comprising a sequence encoding at least a portion of the antibody or TCR or a reverse complement thereof as a template, thereby generating an extended capture probe; and optionally generating a complement of the extended capture probe. The methods can further include amplifying the second barcoded polynucleotide with a first primer that hybridizes to a functional sequence of the capture probe or reverse complement thereof and a second primer that hybridizes to a nucleic acid sequence encoding a variable region of the antibody or TCR cell or reverse complement thereof, wherein the first primer flanks the spatial barcode of the second barcoded polynucleotide.

In some embodiments, the nucleic acid comprising a sequence encoding at least a portion of the antibody or TCR or a reverse complement thereof comprises a sequence encoding the variable region and constant region of the antibody or TCR. In some embodiments, generating a second barcoded polynucleotide in step (e) comprises extending the capture probe using the nucleic acid comprising the sequence encoding the variable region and constant region of the antibody or TCR as a template, thereby generating an extended capture probe; and amplifying the extended capture probe to provide a nucleic acid library. The methods can further include circularizing a member of the nucleic acid library to generate a circularized nucleic acid, and amplifying the circularized nucleic acid using a first primer and a second primer to generate a double-stranded member of the nucleic acid library lacking all, or a portion of, the sequence encoding a constant region of the antibody or TCR.

In some embodiments, said generating a second barcoded polynucleotide in step (e) comprises (i) contacting the biological sample with a first primer that hybridizes to the nucleic acid comprising a sequence encoding at least a portion of the ABM or a reverse complement thereof, wherein the first primer comprises a functional domain; (ii) extending the first primer using the nucleic acid comprising a sequence encoding at least a portion of the ABM or a reverse complement thereof as a template to generate an extension product; (iii) adding a polynucleotide sequence comprising at least three nucleotides to the 3′ end of the extension product; (iv) hybridizing a second primer to the polynucleotide sequence comprising at least three nucleotides of the extension product of (iii), wherein the second primer comprises a capture sequence; (v) extending the extension product using the second primer as a template, thereby incorporating a complement of the capture sequence into the extension product; (vi) hybridizing the complement of the capture sequence of the extension product to the second capture domain of the second capture probe; and (vii) extending the 3′ end of the extension product of (v) using the capture probe as a template, thereby generating an extended capture product.

In some embodiments, the first primer hybridizes to a region of the nucleic acid encoding a constant region of the ABM.

In some embodiments, said generating a first barcoded polynucleotide in step (d) comprises extending the first reporter oligonucleotide using the first capture probe as a template, thereby providing an extended first reporter oligonucleotide. In some embodiments, the methods further include amplifying the extended first reporter oligonucleotide.

In some embodiments, the methods further include generating a third barcoded polynucleotide or a plurality of third barcoded polynucleotides comprising (i) the second reporter barcode sequence or reverse complement thereof, and (ii) the first or second spatial barcode or reverse complement thereof, and optionally using the third barcoded polynucleotide or plurality of third barcoded polynucleotides to identify the ABM as having bound to the non-target antigen coupled to the second reporter oligonucleotide. In some embodiments, the determining and/or identifying in step (f) comprises determining sequences of the first barcoded polynucleotide and the second barcoded polynucleotide, and optionally determining a sequence of the third barcoded polynucleotide. In any of the foregoing, the determining can be performed by sequencing.

In some embodiments, the method includes identifying the ABM based on the determined sequence of the second barcoded polynucleotide. The determined sequence can include a nucleotide sequence. In some embodiments, the determined sequence can include an amino acid sequence encoded by the nucleotide sequence.

In some embodiments, the binding affinity of the ABM to the target antigen is assessed based on the determined sequence of the first barcoded polynucleotide.

In some embodiments, the nucleic acid comprising a sequence encoding at least a portion of the ABM or a reverse complement thereof is RNA or DNA. In some embodiments, the RNA is mRNA and the DNA is genomic DNA or cDNA.

The methods further can further involve fixing the biological sample. Fixing the biological sample can include the use of a fixative selected from the group consisting of: ethanol, methanol, acetone, formaldehyde, paraformaldehyde-Triton, glutaraldehyde, and combinations thereof.

In some embodiments, the methods further comprise staining the biological sample. The staining can include use of eosin and/or hematoxylin. In some embodiments, the staining comprises the use of a detectable label selected from the group consisting of a radioisotope, a fluorophore, a chemiluminescent compound, a bioluminescent compound, or a combination thereof.

In some embodiments, the method further includes imaging the biological sample. The imaging can involve one or more of expansion microscopy, bright field microscopy, dark field microscopy, phase contrast microscopy, electron microscopy, fluorescence microscopy, reflection microscopy, interference microscopy and confocal microscopy.

The methods can further include a step of permeabilizing the biological sample. The permeabilizing can involve the use of an organic solvent, a detergent, an enzyme, or a combination thereof. In some embodiments, the permeabilizing comprises the use of an endopeptidase, wherein the endopeptidase is pepsin or proteinase K, a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof.

In some embodiments, the biological sample is from a vertebrate subject. For example, the vertebrate subject can be a mammalian subject, such as a human. In some embodiments, the biological sample is a tissue sample. The tissue sample can be a fixed tissue sample, such as a formalin-fixed paraffin embedded tissue sample, a paraformaldehyde fixed tissue sample, a methanol fixed tissue sample, or an acetone fixed tissue sample. In some embodiments, the tissue sample is a fresh frozen tissue sample, such as a fresh frozen tissue section.

In some embodiments, the biological sample is a tissue section. The tissue section can be a fixed tissue section, such as a formalin-fixed paraffin embedded tissue section, a paraformaldehyde fixed tissue section, a methanol fixed tissue section, or an acetone fixed tissue section.

In some embodiments, the biological sample is a diseased tissue sample and/or a tissue sample derived from a subject having a disease or disorder. The disease or disorder can be cancer, an autoimmune disease, a neurodegenerative disease, an infectious disease, or an inflammatory disease. In some embodiments, the target antigen is associated with the disease or disorder. For example, the target antigen can be a cancer antigen or a coronavirus derived antigen. In some embodiments, the target antigen is a peptide.

Also provided herein is an isolated ABM identified by any of the foregoing methods and pharmaceutical compositions thereof. For example, disclosed herein is a pharmaceutical composition comprising an isolated ABM identified according to a method disclosed herein, or a cell expressing the ABM, and a pharmaceutically acceptable excipient.

Further provided herein are kits for the practice of a method described herein. In some embodiments, a kit for identifying and/or characterizing an ABM or fragment thereof having binding affinity for an antigen includes: (a) a plurality of target antigens and non-target antigens, wherein each of the target antigens and non-target antigens comprise a reporter oligonucleotide comprising (i) a barcode sequence that identifies the antigen or non-antigen, and (ii) a capture handle sequence; (b) a spatial array comprising a first capture probe comprising (i) a barcode sequence, and (ii) a first capture domain, and a second capture probe comprising (i) a barcode sequence, and (ii) a second capture domain. In some embodiments, the kit further includes instructions for performing a method disclosed herein.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A-6C schematically illustrate examples of labelling agents.

FIG. 7 depicts an example of a barcode carrying bead.

FIG. 8 schematically depicts an example workflow for processing nucleic acid molecules.

FIGS. 9A-9B are schematic representations of non-limiting examples of the labelling reagents in accordance with some embodiments of the disclosure.

FIG. 10 is a schematic diagram depicting an exemplary sandwiching process between a first substrate comprising a biological sample and a second substrate comprising a spatially barcoded array.

FIGS. 11A-11B are schematic diagrams depicting exemplary sandwiching embodiments. FIG. 11A shows an exemplary sandwiching process where a first substrate including a biological sample and a second substrate are brought into proximity with one another and a liquid reagent drop is introduced on the second substrate in proximity to the capture probes and in between the biological sample. FIG. 11B shows a fully formed sandwich configuration creating a chamber formed from one or more spacers, the first substrate, and the second substrate including spatially barcoded capture probes.

FIGS. 12A-12B are schematic illustrations of capture probes. FIG. 12A shows an exemplary capture probe with a capture sequence (also referred to as “capture domain”) complementary to a sequence encoding a constant region of an analyte (e.g., an antigen-binding molecule). FIG. 12B shows an exemplary capture probe with a poly(dT) capture domain.

FIG. 13 shows an exemplary enrichment strategy with a Read1 primer and a primer(s) complementary to a region of a nucleic acid analyte encoding a variable region of an antigen-binding molecule (e.g., antibody or T-cell receptor).

FIG. 14 shows an exemplary sequencing strategy with a sequencing handle (P5) and a custom sequencing primer complementary to a portion of a nucleic acid encoding or corresponding to the constant region of an antigen-binding molecule.

FIG. 15 shows an exemplary nucleic acid library preparation method to remove a portion of a nucleic acid analyte sequence via double circularization of a member of a nucleic acid library.

FIG. 16 depicts another exemplary workflow for processing a double-stranded circularized nucleic acid product.

FIG. 17 shows an exemplary nucleic acid library preparation method to remove all or a portion of a nucleic acid analyte encoding a constant region of an ABM from a member of a nucleic acid library via circularization.

FIG. 18 shows an exemplary nucleic acid library method to reverse the orientation of an analyte sequence in a member of a nucleic acid library.

FIG. 19 is a schematic diagram showing an exemplary feature comprising an attached first probe comprising a poly(T) capture domain and second probe comprising a poly(GI) capture domain.

FIGS. 20A-20C are workflow schematics illustrating exemplary steps for generating a spatially barcoded sample for analysis and for use in further steps of the methods described herein (FIG. 20A), specific binding of the extended first probe with the second probe (FIG. 20B), and generating a spatially barcoded sample for analysis that allows for the sequencing of the target nucleic acid from both the 3′ end and the 5′ end (FIG. 20C).

FIG. 21 is a schematic diagram showing reverse transcription of a target nucleic acid with a first primer and the addition of the complement of a capture sequence into an extension product which is capable of hybridizing to a capture domain of a capture probe.

FIG. 22 is a schematic diagram showing capture and extension on an array of the extension product (e.g., cDNA product) shown in FIG. 21 and extension of the capture probe and the captured extension product (e.g., cDNA product) followed by release of the extended capture product.

FIGS. 23A-23B are mouse brain images showing fluorescently labeled cDNA post reverse transcription (FIG. 23A) and post permeabilization and cDNA extension (FIG. 23B).

FIGS. 24A-24B show mouse brain images. FIG. 24A shows a brightfield image and FIG. 24B shows fluorescently labeled extended cDNA generated by extension in the presence of Cy3-dCTPs.

FIGS. 25A-25C shows spatial gene expression clusters (FIG. 25A), the corresponding t-SNE plot (FIG. 25B), and spatial gene expression heat map (FIG. 25C).

FIGS. 26A-26D show spatial gene expression clustering with a first primer including a poly(T) sequence (FIG. 26A) and the corresponding t-SNE plot (FIG. 26B) and spatial gene expression clustering with a first primer including a random decamer (FIG. 26C) and the corresponding t-SNE plot (FIG. 26D).

FIG. 27 shows fluorescently labeled extended cDNA post permeabilization and cDNA extension in mouse brain tissue using a template switch ribonucleotide with an alternative handle.

FIGS. 28A-28B are graphs showing correlation between fresh frozen capture using standard Visium spatial gene expression (10× Genomics) and spatial 5′ end capture (FIG. 28A) and a graph showing normalized position of each mapped read within the full-length transcript and confirming successful 5′ enrichment with a primer including a random decamer (FIG. 28B).

### DETAILED DESCRIPTION OF THE DISCLOSURE

The present disclosure generally relates to, inter alia, methods, compositions and kits for the identification and characterization of antigen-binding molecules (e.g., antibodies, TCRs) produced by immune cells (e.g., B-cells, T-cells) in a biological sample, using spatial immune profiling methodologies. The disclosed methods, compositions and kits are particularly useful for generation of recombinant antigen-binding molecules (ABMs) with desired properties and for their use in various downstream applications. As described in greater detail below, the approaches described in the present disclosure allow for the identification and isolation of ABMs using “barcode-enabled antigen mapping by sequencing” (BEAM-seq). In particular, by contacting biological samples with antigens labeled with 1) nucleic acid (e.g., DNA) oligonucleotides, 2) biotin-strepavidin or a functional equivalent, optionally, 3) fluorophores (e.g., PE or APC), and optionally tetramerizing the antigens as needed, it is possible to successfully identify ABMs (e.g., antibodies or TCRs) that specifically bind the antigen, while excluding non-specific ABMs that, for example, bind biotin/strepavidin, the fluorophores, or an irrelevant control antigen (e.g., human serum albumin). The BEAM-seq workflows disclosed herein can identify ABMs within a much shorter timeframe than traditional discovery approaches. In addition, the BEAM-seq workflows disclosed herein allows direct capture of full-length ABM (e.g., antibody) sequences, facilitating rapid expression of the native ABM.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

The term “barcode” is used herein to refer to a label, or identifier, that conveys or is capable of conveying information (e.g., information about an analyte in a sample, a bead, and/or a nucleic acid barcode molecule). A barcode can be part of an analyte or nucleic acid barcode molecule, or independent of an analyte or nucleic acid barcode molecule. A barcode can be attached to an analyte or nucleic acid barcode molecule in a reversible or irreversible manner. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for or facilitates identification and/or quantification of individual sequencing-reads. In some embodiments, a barcode can be configured for use as a fluorescent barcode. For example, in some embodiments, a barcode can be configured for hybridization to fluorescently labeled oligonucleotide probes. Barcodes can be configured to spatially resolve molecular components found in biological samples, for example, at single-cell resolution (e.g., a barcode can be or can include a “spatial barcode”). In some embodiments, a barcode includes two or more sub-barcodes that together function as a single barcode. For example, a polynucleotide barcode can include two or more polynucleotide sequences (e.g., sub-barcodes). In some embodiments, the two or more sub-barcodes are separated by one or more non-barcode sequences. In some embodiments, the two or more sub-barcodes are not separated by non-barcode sequences.

In some embodiments, a barcode can include one or more unique molecular identifiers (UMIs). Generally, a unique molecular identifier is a contiguous nucleic acid segment or two or more non-contiguous nucleic acid segments that function as a label or identifier for a particular analyte, or for a nucleic acid barcode molecule that binds a particular analyte (e.g., mRNA) via the capture sequence. A UMI can include one or more specific polynucleotides sequences, one or more random nucleic acid and/or amino acid sequences, and/or one or more synthetic nucleic acid and/or amino acid sequences. In some embodiments, the UMI is a nucleic acid sequence that does not substantially hybridize to analyte nucleic acid molecules in a biological sample. In some embodiments, the UMI has less than 80% sequence identity (e.g., less than 70%, 60%, 50%, or less than 40% sequence identity) to the nucleic acid sequences across a substantial part (e.g., 80% or more) of the nucleic acid molecules in the biological sample. These nucleotides can be completely contiguous, i.e., in a single stretch of adjacent nucleotides, or they can be separated into two or more separate subsequences that are separated by 1 or more nucleotides.

The term “analyte carrier,” as used herein, generally refers to a discrete biological system derived from a biological sample. The analyte carrier may be or comprise a biological particle. The analyte carrier, e.g., biological particle, may be a macromolecule. The analyte carrier, e.g., biological particle, may be a small molecule. The analyte carrier, e.g., biological particle, may be a virus, e.g., a phage. The analyte carrier, e.g., biological particle, may be a cell or derivative of a cell. The analyte carrier, e.g., biological particle, may be an organelle. The analyte carrier, e.g., biological particle, may be a rare cell from a population of cells. The analyte carrier, e.g., biological particle, may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The analyte carrier, e.g., biological particle, may be a constituent of a cell. The analyte carrier, e.g., biological particle, may be or may include DNA, RNA, organelles, proteins, or any combination thereof. The analyte carrier, e.g., biological particle, may be or may include a matrix (e.g., a gel or polymer matrix) including a cell or one or more constituents from a cell (e.g., cell bead), such as DNA, RNA, organelles, proteins, or any combination thereof, from the cell. The analyte carrier, e.g., biological particle, may be obtained from a tissue of a subject. The analyte carrier, e.g., biological particle, may be a hardened cell. Such hardened cell may or may not include a cell wall or cell membrane. The analyte carrier, e.g., biological particle, may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when including a gel or polymer matrix.

An “equivalent amino acid residue” refers to an amino acid residue capable of replacing another amino acid residue in a polypeptide without substantially altering the structure and/or functionality of the polypeptide. Equivalent amino acids thus have similar properties such as bulkiness of the side-chain, side chain polarity (polar or non-polar), hydrophobicity (hydrophobic or hydrophilic), pH (acidic, neutral or basic) and side chain organization of carbon molecules (aromatic/aliphatic). As such, “equivalent amino acid residues” can be regarded as “conservative amino acid substitutions” to each other.

Within the meaning of the term “equivalent amino acid substitution” as applied herein, one amino acid may be substituted for another without substantially altering the structure and/or functionality of the polypeptide. Exemplary equivalent or conserved amino acid substitutions are within the groups of amino acids indicated herein below:
i) Amino acids having polar side chains (Asp, Glu, Lys, Arg, His, Asn, Gln, Ser, Thr, Tyr, and Cys);
ii) Amino acids having non-polar side chains (Gly, Ala, Val, Leu, Ile, Phe, Trp, Pro, and Met);
iii) Amino acids having aliphatic side chains (Gly, Ala Val, Leu, Ile);
iv) Amino acids having cyclic side chains (Phe, Tyr, Trp, His, Pro);
v) Amino acids having aromatic side chains (Phe, Tyr, Trp);
vi) Amino acids having acidic side chains (Asp, Glu);
vii) Amino acids having basic side chains (Lys, Arg, His);
viii) Amino acids having amide side chains (Asn, Gln);
ix) Amino acids having hydroxy side chains (Ser, Thr);
x) Amino acids having sulphur-containing side chains (Cys, Met);
xi) Neutral, weakly hydrophobic amino acids (Pro, Ala, Gly, Ser, Thr);
xii) Hydrophilic, acidic amino acids (Gin, Asn, Glu, Asp); and.
xiii) Hydrophobic amino acids (Leu, Ile, Val).

In some embodiments, a Point Accepted Mutation (PAM) matrix is used to determine equivalent amino acid substitutions. In some embodiments, a BLOck SUbstitution Matrix (BLOSUM) is used to determine equivalent amino acid substitutions.

As used herein, “isolated” antigen-binding molecules (e.g., antibodies, TCRs, or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or antigen-binding fragments.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant proteins and nucleic acids include proteins and nucleic acids produced by laboratory methods. Recombinant proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, a “subject” or an “individual” includes animals, such as human (e.g., human individuals) and non-human animals. The term “non-human animals” includes all vertebrates, e.g., mammals, e.g., rodents, e.g., mice, non-human primates, and other mammals, such as e.g., rat, mouse, cat, dog, cow, pig, sheep, horse, goat, rabbit; and non-mammals, such as amphibians, reptiles, etc. A subject can be a mammal, preferably a human or humanized animal, e.g., an animal with humanized VDJC loci. The subject may be non-human animals with humanized VDJC loci and knockouts of a target of interest. The subject may be in need of prevention and/or treatment of a disease or disorder such as viral infection or cancer. The subject may have a viral infection, e.g., a coronavirus infection, or be predisposed to developing an infection. Subjects predisposed to developing an infection, or subjects who may be at elevated risk for contracting an infection (e.g., of coronavirus), include subjects with compromised immune systems because of autoimmune disease, subjects receiving immunosuppressive therapy (for example, following organ transplant), subjects afflicted with human immunodeficiency syndrome (HIV) or acquired immune deficiency syndrome (AIDS), subjects with forms of anemia that deplete or destroy white blood cells, subjects receiving radiation or chemotherapy, or subjects afflicted with an inflammatory disorder. Additionally, subjects of very young (e.g., 5 years of age or younger) or old age (e.g., 65 years of age or older) are at increased risk. Moreover, a subject may be at risk of contracting a viral infection due to proximity to an outbreak of the disease, e.g., subject resides in a densely-populated city or in close proximity to subjects having confirmed or suspected infections of a virus, or choice of employment, e.g. hospital worker, pharmaceutical researcher, traveler to infected area, or frequent flier.

A “variant” of a polypeptide, such as an immunoglobulin chain (e.g., VH, VL, HC, or LC), refers to a polypeptide comprising an amino acid sequence that has at least about 70-99.9% (e.g., 70%, 72%, 74%, 75%, 76%, 79%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9%) sequence identity or similarity to a referenced amino acid sequence that is set forth herein. In some embodiments, the term “percent identity,” as used herein in the context of two or more proteins, refers to two or more sequences or subsequences that are the same or have a specified percentage of amino acids that are the same, e.g., about 70%, 72%, 74%, 75%, 76%, 79%, 80%, 81%, 82%, 83%, 84%, 85%, 86%, 87%, 88%, 89%, 90%, 91%, 92%, 93%, 94%, 95%, 96%, 97%, 98%, 9%9, 99.5%, 99.9%, or higher identity over a specified region, when compared and aligned for maximum correspondence over a comparison window or designated region as measured using a BLAST or BLAST 2.0 sequence comparison algorithms with default parameters described below, or by manual alignment and visual inspection. See, e.g., the NCBI web site at ncbi.nlm.nih.gov/BLAST. In some embodiments, this definition also refers to, or may be applied to, the complement of a query sequence. In some embodiments, this definition includes sequence comparison performed by a BLAST algorithm wherein the parameters of the algorithm are selected to give the largest match between the respective sequences over the entire length of the respective reference sequences. In some embodiments, this definition also includes sequences that have deletions and/or additions, as well as those that have substitutions. Sequence identity can be calculated over a region that is at least about 20 amino acids or nucleotides in length, or over a region that is 10-100 amino acids or nucleotides in length, or over the entire length of a given sequence. In some embodiments, sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403), IgBLAST, and IMGT/V-QUEST. In some embodiments, sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Additional methodologies that can suitably be utilized to determine structural similarity or identity amino acid sequences include those relying on position-specific structure-scoring matrix (P3SM) that incorporates structure-prediction scores from Rosetta, as well as those based on a length-normalized edit distance as described previously in, e.g., Setliff et al., Cell Host & Microbe 23(6), May 2018.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value ±up to 10%, up to ±5%, or up to ±1%.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Methods of the Disclosure

### Methods and Systems for Spatial Analysis of a Biological Sample

The present disclosure provides methods for the identification and/or characterization of ABMs obtained from biological samples using spatial profiling. In particular, spatial profiling employs approaches that allow for characterization of the identity and spatial location of a biomolecule, such as an ABM, within a biological sample. A general description of spatial profiling or analysis as used in the disclosed methods for the identification and/or characterization of ABMs is provided below.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode, a ligation product) associated with the intermediate agent. Detection of the intermediate agent is therefore indicative of the analyte in the biological sample (e.g., cell, tissue section, etc.).

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198; U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621; WO 2018/091676, WO 2020/176788, WO 2022/061152, WO 2021/252747, PCT/US2021/061401; Rodriques et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), both of which are available at the 10× Genomics Support Documentation website, and can be used herein in any combination. Further non-limiting aspects of spatial analysis methodologies and compositions are described herein.

Some general terminologies that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Examples of nucleic acid analytes include, but are not limited to, DNA (e.g., genomic DNA, cDNA) and RNA, including coding and non-coding RNA (e.g., mRNA, rRNA, tRNA, ncRNA).

Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is generally obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

In some instances, a capture probe and a nucleic acid analyte (or any other nucleic acid to nucleic acid interaction) occurs because the sequences of the two nucleic acids are substantially complementary to one another. By “substantial,” “substantially” and the like, two nucleic acid sequences can be complementary when at least 60% of the nucleotide residues of one nucleic acid sequence are complementary to nucleotide residues in the other nucleic acid sequence. The complementary residues within a particular complementary nucleic acid sequence need not always be contiguous with each other, and can be interrupted by one or more non-complementary residues within the complementary nucleic acid sequence. In some embodiments, at least 60%, but less than 100%, of the residues of one of the two complementary nucleic acid sequences are complementary to residues in the other nucleic acid sequence. In some embodiments, at least 70%, 80%, 90%, 95% or 99% of the residues of one nucleic acid sequence are complementary to residues in the other nucleic acid sequence. Sequences are said to be “substantially complementary” when at least 60% (e.g., at least 70%, at least 80%, or at least 90%) of the residues of one nucleic acid sequence are complementary to residues in the other nucleic acid sequence.

In some embodiments, the biological sample is mounted on a first substrate and the substrate comprising the array of capture probes is a second substrate. During this process, one or more analytes or analyte derivatives (e.g., intermediate agents) are released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

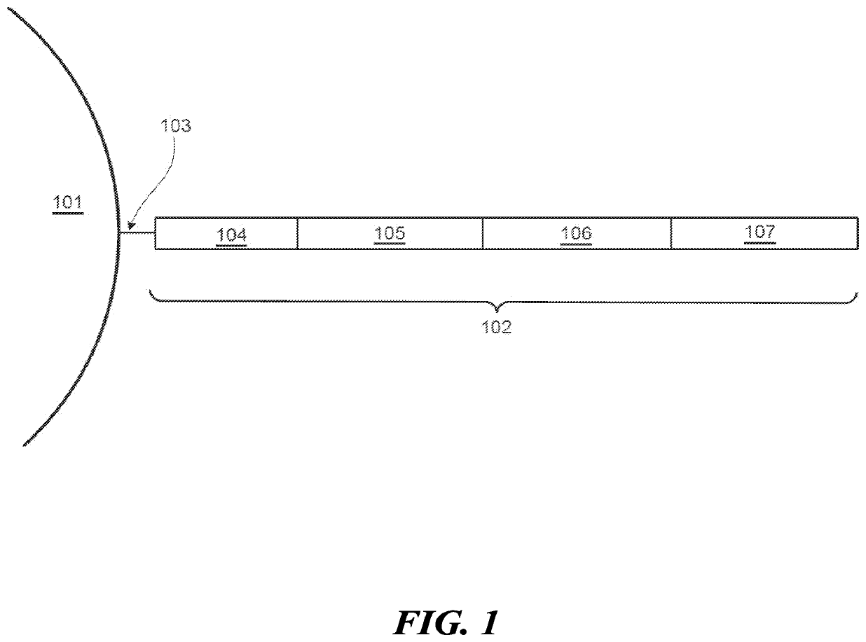

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent or labeling agent. The capture domain can have a sequence complementary to a splint oligonucleotide. A splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

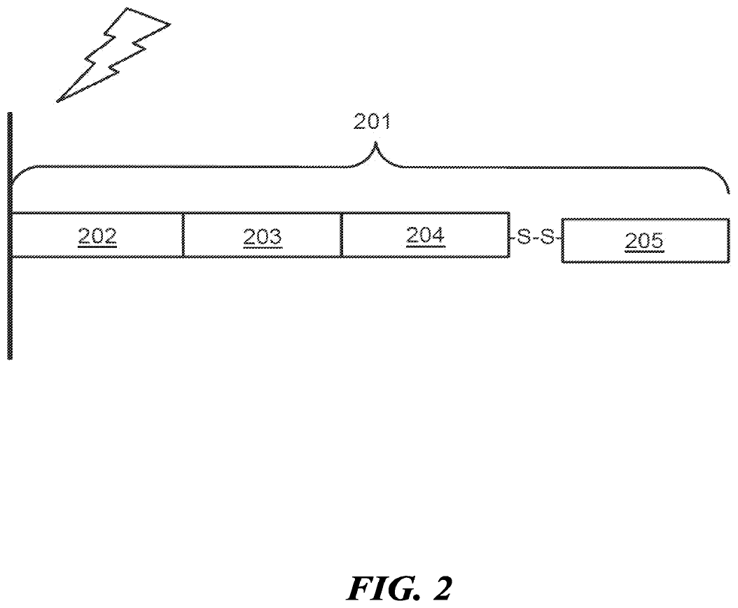

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

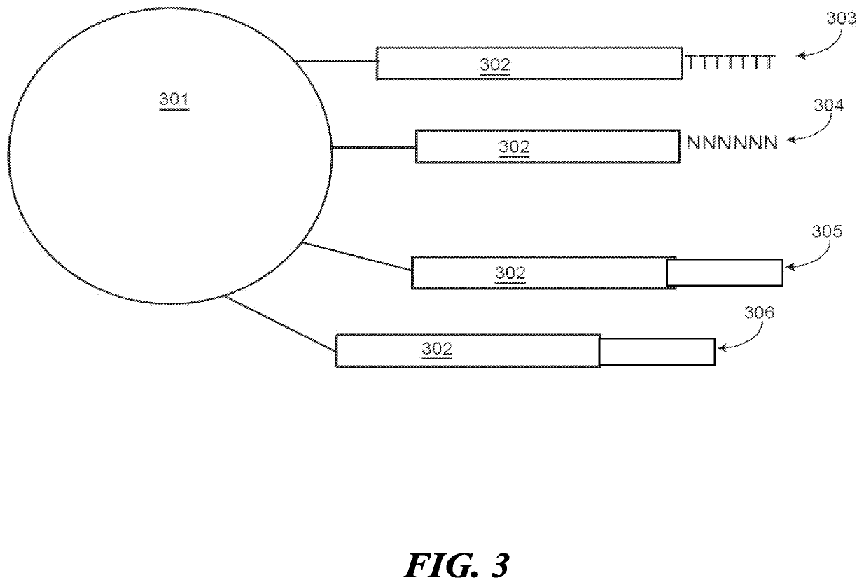

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

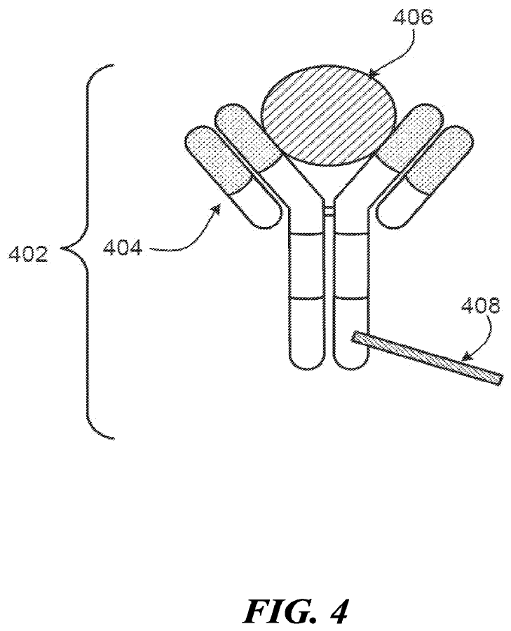

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408 which serves to identify the analyte binding moiety, and a capture domain which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

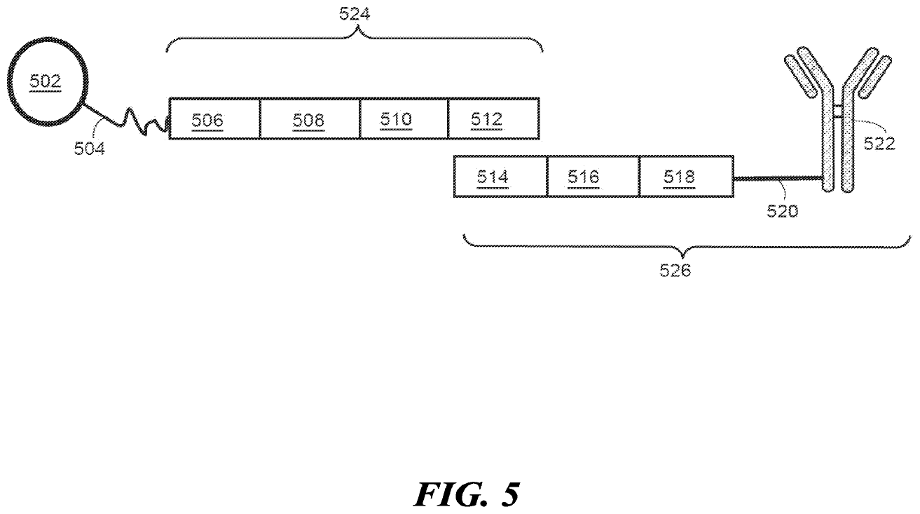

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can be affixed 504 to a feature such as a bead 502. The capture probe can also include a capture domain 512 that is capable of binding to an analyte capture agent 526. The analyte-binding moiety barcode domain of the analyte capture agent 526 can include a functional sequence 518, analyte binding moiety barcode 516, and an analyte capture sequence 514 that is capable of binding (e.g., hybridizing) to the capture domain 512 of the capture probe 524. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the analyte-binding moiety barcode domain (e.g., including the functional sequence 518, analyte binding barcode 516, and analyte capture sequence/capture handle sequence 514) to couple to the analyte binding moiety 522. In some embodiments, the linker is a cleavable linker. In some embodiments, the cleavable linker is a photo-cleavable linker, a UV-cleavable linker, or an enzyme cleavable linker. In some instances, the cleavable linker is a disulfide linker. A disulfide linker can be cleaved by use of a reducing agent, such as dithiothreitol (DTT), Beta-mercaptoethanol (BME), or Tris (2-carboxyethyl) phosphine (TCEP).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In general, spatial transcriptomics methods comprise a spatially-barcoded array populated with capture probes (as described further herein) that is contacted with a biological sample, the biological sample is permeabilized thereby allowing the analytes in the biological sample to migrate away from the sample and toward the array, for example via passive (e.g., gravitational) or active (e.g., electrophoretic) forces. The analyte hybridizes with a capture domain on a capture probe on the spatially-barcoded array. Once the analyte hybridizes/is bound to the capture domain of the capture probe, the capture probe is extended, using the capture analyte as a template, and the sequence of the extended capture probe, or a complement thereof, is analyzed to obtain spatially-resolved analyte information. The biological sample can also be optionally removed from the array following analyte capture on the array. In some instances, the spatially-barcoded array populated with capture probes (as described further herein) is contacted with a biological sample, and the biological sample is permeabilized, allowing the analyte to migrate away from the sample and toward the array. The analyte interacts with a capture probe on the spatially-barcoded array.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, generating an extended capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

As used herein, “an extension product” refers to an analyte, such as RNA (e.g., mRNA), that has been reverse transcribed (e.g., with a reverse transcriptase) to generate cDNA. In some embodiments, the cDNA is hybridized to the RNA (e.g., RNA/cDNA duplex). In some embodiments, the cDNA is hybridized to the mRNA. In some embodiments, the extension product is further extended by adding a polynucleotide sequence (e.g., a heteropolynucleotide sequence, a homopolynucleotide sequence) to the 3′ end of the extension product (e.g., cDNA). For example, a reverse transcriptase or a terminal transferase can add at least three nucleotides (e.g., a polynucleotide sequence, such as 5′-CCC-3′) to the 3′ end of the extension product in a template-independent manner. In some embodiments, the extension product is further extended when a second primer hybridizes to the polynucleotide sequence and the extension product is further extended using the second primer as a template. In some embodiments, the second primer includes a capture sequence which is incorporated (e.g., a complement thereof) into the sequence of the extension product. In some embodiments, the RNA (e.g., mRNA) is removed (e.g., by digestion) from the extension product. In such embodiments, the term extension product also refers to a single-stranded DNA (e.g., cDNA) product that includes a complement of the target nucleic acid (e.g., RNA, mRNA).

As used herein, “an extended capture product” refers to an extension product that has been captured on a spatial array and extended using the capture probe as a template. For example, an extension product, as described herein, after capture by a capture probe can be extended to include a capture sequence, or a complement thereof, that is capable of hybridizing to a capture domain of a capture probe. In some embodiments, when the extension product hybridizes to the capture domain of the capture probe, an end of the extension product (e.g., a 3′ end) can be extended to generate the extended capture product. In such examples, the extended capture product includes the domains (e.g., a UMI, a spatial barcode, one or more functional domains, etc.) present in the capture probe on the spatial array. In some embodiments, the extended capture product is released from the capture probe and collected for downstream applications, such as amplification and sequencing.

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes using the captured analyte or intermediate agent as a template, sequencing (e.g., of a cleaved extended capture probe and/or a nucleic acid molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

As a further examples, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Generally, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof), such as an extension product, can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020). In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2021/102039 and/or U.S. patent application Ser. No. 16/951,864, each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843, each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### Sandwiching Processes

In some embodiments of the methods disclosed herein for the identification and/or characterization of ABMs obtained from biological samples using spatial profiling, one or more analytes from the biological sample are released from the biological sample and migrate to a substrate comprising an array of capture probes for attachment to the capture probes of the array. In some embodiments, the release and migration of the analytes to the substrate comprising the array of capture probes occurs in a manner that preserves the original spatial context of the analytes in the biological sample. In some embodiments, the biological sample is mounted on a first substrate and the substrate comprising the array of capture probes is a second substrate. In some embodiments, the method is facilitated by a sandwiching process. Sandwiching processes are described in, e.g., US. Patent Application Pub. No. 20210189475, PCT Patent Application Pub. No. WO 2021/252747 A1, and WO 2022/061152, which are hereby incorporated by reference. In some embodiments, the sandwiching process may be facilitated by a device, sample holder, sample handling apparatus, or system described in, e.g., US. Patent Application Pub. No. 20210189475, PCT/US2021/036788, or PCT/US2021/050931.

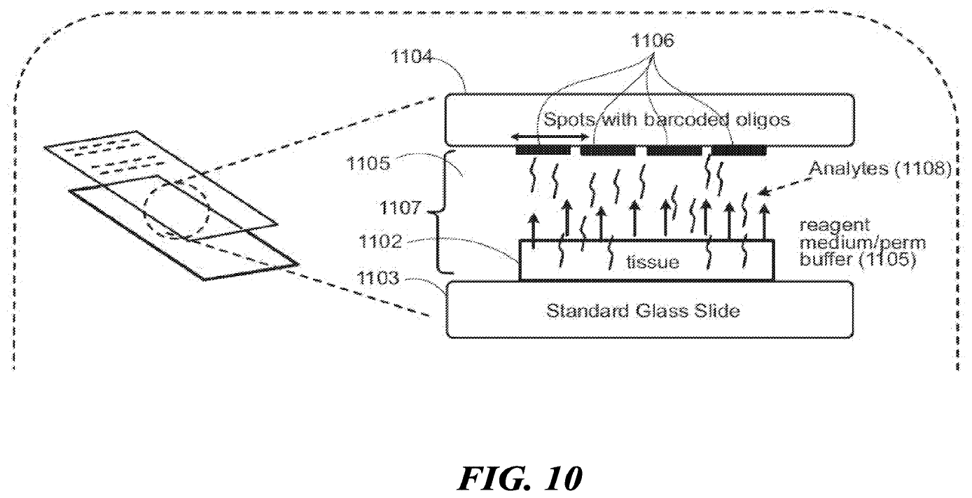

FIG. 10 is a schematic diagram depicting an exemplary sandwiching process between a first substrate comprising a biological sample (e.g., a tissue section 1102 on a slide 1103) and a second substrate comprising a spatially barcoded array, e.g., a slide 1104 that is populated with spatially-barcoded capture probes 1106. During the exemplary sandwiching process, the first substrate is aligned with the second substrate, such that at least a portion of the biological sample is aligned with at least a portion of the array (e.g., aligned in a sandwich configuration). As shown, the second substrate (e.g., slide 1104) is in a superior position to the first substrate (e.g., slide 1103). In some embodiments, the first substrate (e.g., slide 1103) may be positioned superior to the second substrate (e.g., slide 1104). A reagent medium 1105 (e.g., permeabilization solution) within a gap 1107 between the first substrate (e.g., slide 303) and the second substrate (e.g., slide 1104) creates a permeabilization buffer which permeabilizes or digests the sample 1102 and the analytes (e.g., mRNA, DNA, reporter oligonucleotides associated with target and non-target antigens) 1108 of the biological sample 1102 may release, actively or passively migrate (e.g., diffuse) across the gap 1107 toward the capture probes 1106, and bind on the capture probes 1106.

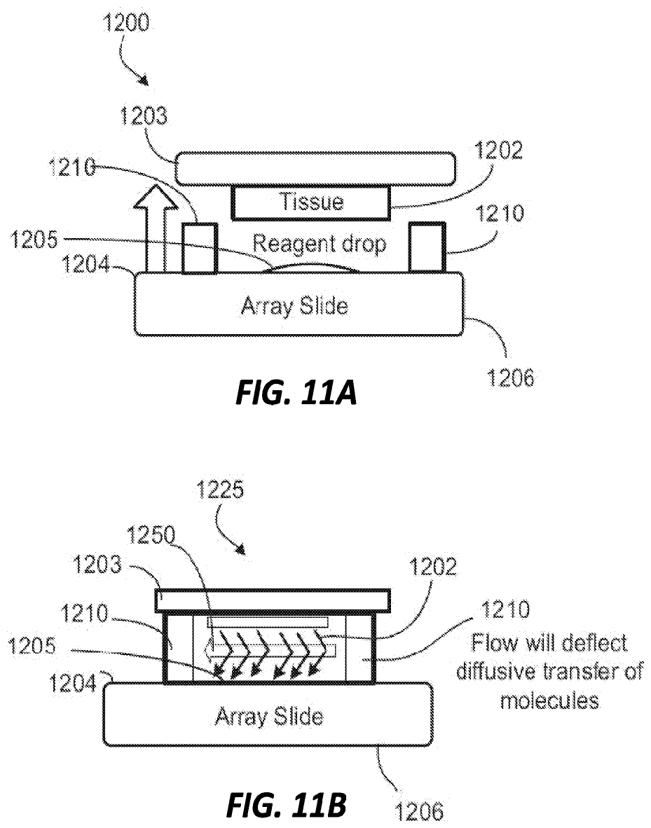

After the analytes (e.g., transcripts) 1108 bind the capture probes 1106, an extension reaction may occur, thereby generating a spatially barcoded library. For example, in the case of mRNA transcripts, reverse transcription may be used to generate a cDNA library associated with a particular spatial barcode. Barcoded cDNA libraries may be mapped back to a specific location on a capture area of the capture probes 1106. This data may be subsequently layered over a high-resolution microscope image of the biological sample (e.g., tissue section), making it possible to visualize the data within the morphology of the biological sample in a spatially-resolved manner. In some embodiments, the extension reaction can be performed separately from the sample handling apparatus described herein that is configured to perform the exemplary sandwiching process illustrated in FIGS. 10 and 11A-11B. The sandwich configuration of the sample 1102, the first substrate (e.g., slide 1103) and the second substrate (e.g., slide 1104) may provide advantages over other methods of spatial analysis and/or analyte capture. For example, the sandwich configuration may reduce a burden of users to develop in house tissue sectioning and/or tissue mounting expertise. Further, the sandwich configuration may decouple sample preparation/tissue imaging from the barcoded array (e.g., spatially-barcoded capture probes 1106) and enable selection of a particular region of interest of analysis (e.g., for a tissue section larger than the barcoded array). The sandwich configuration also beneficially allows spatial analysis without having to place a biological sample (e.g., tissue section) 1102 directly on the second substrate (e.g., slide 1104).

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism (also referred to herein as an adjustment mechanism) of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the analyte from the biological sample.

The sandwiching process methods described above can be implemented using a variety of hardware components. For example, the sandwiching process methods can be implemented using a sample holder (also referred to herein as a support device, a sample handling apparatus, and an array alignment device).

In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further includes an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

In the example sandwich maker workflows described herein, the reagent medium (e.g., liquid reagent medium, permeabilization solution 1205) may fill a gap (e.g., the gap 1207) between a first substrate (e.g., slide 1203) and a second substrate (e.g., slide 1204 with barcoded capture probes 1206) to warrant or enable transfer of target molecules with spatial information. Various filling methods that may suppress bubble formation and suppress undesirable flow of transcripts and/or target molecules or analytes can be used. Robust fluidics in the sandwich making described herein may preserve spatial information by reducing or preventing deflection of molecules as they move from the tissue slide to the capture slide.

FIG. 11A shows an exemplary sandwiching process 1200 where a first substrate (e.g., slide 1203), including a biological sample 1202 (e.g., a tissue section), and a second substrate (e.g., slide 1204 including spatially barcoded capture probes 1206) are brought into proximity with one another. As shown in FIG. 11A, a liquid reagent drop (e.g., permeabilization solution 1205) is introduced on the second substrate in proximity to the capture probes 1206 and in between the biological sample 1202 and the second substrate (e.g., slide 1204 including spatially barcoded capture probes 1206). The permeabilization solution 1205 may release analytes that can be captured by the capture probes 1206 of the array. As further shown, one or more spacers 1210 may be positioned between the first substrate (e.g., slide 1203) and the second substrate (e.g., slide 124 including spatially barcoded capture probes 1206). The one or more spacers 1210 may be configured to maintain a separation distance between the first substrate and the second substrate. While the one or more spacers 1210 is shown as disposed on the second substrate, the spacer may additionally or alternatively be disposed on the first substrate.

In some embodiments, the one or more spacers 1210 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 11B shows a fully formed sandwich configuration creating a chamber 1250 formed from the one or more spacers 1210, the first substrate (e.g., the slide 1203), and the second substrate (e.g., the slide 1204 including spatially barcoded capture probes 1206) in accordance with some example implementations. In the example of FIG. 11B, the liquid reagent (e.g., the permeabilization solution 1205) fills the volume of the chamber 1250 and may create a permeabilization buffer that allows analytes (e.g., mRNA, DNA, reporter oligonucleotides associated with target and non-target antigens) to diffuse from the biological sample 1202 toward the capture probes 1206 of the second substrate (e.g., slide 1204). In some aspects, flow of the permeabilization buffer may deflect transcripts and/or molecules from the biological sample 1202 and may affect diffusive transfer of analytes for spatial analysis. A partially or fully sealed chamber 1250 resulting from the one or more spacers 1210, the first substrate, and the second substrate may reduce or prevent flow from undesirable convective movement of transcripts and/or molecules over the diffusive transfer from the biological sample 1202 to the capture probes 1206.

In some instances, the first substrate and the second substrate are arranged in an angled sandwich assembly as described herein. For example, during the sandwiching of the two substrates (e.g., the slide 1203 and the slide 1204), an angled closure workflow may be used to suppress or eliminate bubble formation.

Methods for Identifying and/or Characterizing ABMs with Binding Affinity to a Target Antigen

In described in more detail below, one aspect of the disclosure relates to approaches and methods for the identification and/or characterization of antigen-binding molecules (ABMs), e.g., antibodies, TCRs, and antigen-binding fragments thereof. In some embodiments, the disclosed methods are used to identify and/or characterize antigen-binding molecules that are derived from or expressed by B-cells or T-cells within biological samples, e.g., by using spatial immune profiling methodologies. Such methods are advantageous to identify and/or generate ABMs (e.g., antibodies, TCRs, and antigen-binding fragments thereof) having a binding specificity for a target antigen, e.g., having the ability to discriminate the target antigen from non-target antigens.

Advantages of the new approaches and methods disclosed herein are numerous. For example, the ABM antibody hits identified via the workflows disclosed herein are likely to have lower developability burden than those identified using display methodologies. It is contemplated that the workflows disclosed herein yield greater numbers of ABMs with superior properties as compared to traditional antibody discovery workflows. Furthermore, the workflows disclosed herein may allow for rapid identification of many ABMs (e.g., antibodies) with broad and robust binding affinity against several antigens, including target antigens and variants of the target antigens. Such workflows are particularly advantageous in the face of rapidly changing disease landscapes where variants of concern evolve over time. In particular, the workflows disclosed herein may be beneficial for identification of patient-specific ABMs, for example by using biological samples (e.g., tissue samples) from a diseased subject in the performance of the disclosed methods. This may allow for rapid identification of potentially therapeutic ABMs (e.g., specific antibodies, TCRs, or fragments thereof) that bind to different antigenic epitopes associated with a disease of interest.

In some embodiments, the methods for identifying and/or characterizing an antigen-binding molecule (ABM) in a biological sample, include: a) contacting the biological sample with a plurality of antigens, wherein the plurality of antigens comprises a target antigen coupled to a first reporter oligonucleotide comprising (i) a first reporter barcode sequence that identifies the target antigen and (ii) a capture handle sequence, and wherein the contacting provides the ABM bound to the target antigen; b) providing an array of capture probes, the array comprising a first capture probe comprising a first spatial barcode and a first capture domain, and a second capture probe comprising a second spatial barcode and a second capture domain; c) hybridizing (i) the capture handle sequence of the first reporter oligonucleotide to the first capture domain of the first capture probe and (ii) a nucleic acid comprising a sequence encoding at least a portion of the ABM or a reverse complement thereof to the second capture domain of the second capture probe; d) generating a first barcoded polynucleotide comprising (i) the first reporter barcode sequence or a reverse complement thereof and (ii) the first spatial barcode or a reverse complement thereof; and e) generating a second barcoded polynucleotide comprising (i) the nucleic acid encoding at least a portion of the ABM or a reverse complement thereof and (ii) the second spatial barcode or a reverse complement thereof.

In some embodiments, the methods further include f) determining the identity and spatial location of the ABM in the biological sample and/or identifying the ABM as having bound to the target antigen using the first and second barcoded polynucleotides.

The plurality of antigens can further include a non-target antigen coupled to a second reporter oligonucleotide, wherein the second reporter oligonucleotide comprises (i) a second reporter barcode sequence that identifies the non-target antigen, and (ii) the capture handle sequence. In some embodiments, the non-target antigen is an antigen to which the ABM is not expected to bind. In some embodiments, the plurality of antigens comprises two or more distinct target antigens, wherein each distinct target antigen is coupled to a reporter oligonucleotide comprising (i) a reporter barcode sequence that identifies the target antigen and (ii) the capture handle sequence.

In some embodiments, the methods further include assessing the binding specificity of the ABM. This can involve, for example: (i) identifying the ABM as having a binding specificity for the target antigen if the ABM binds to the target antigen and does not significantly bind the non-target antigen, or (ii) identifying the ABM as non-specific for the target antigen if the ABM significantly binds to the non-target antigen.

In some embodiments, the array of capture probes comprises a plurality of the first capture probe and a plurality of the second capture probe. In some embodiments, the capture handle sequence of the first reporter oligonucleotide is partially or fully complementary to the first capture domain of the first capture probe. In some embodiments, the first spatial barcode of the first capture probe is identical to the second spatial barcode of the second capture probe. In some embodiments, the first spatial barcode of the first capture probe is different from the second spatial barcode of the second capture probe.

In some embodiments, the target antigen and first reporter oligonucleotide are indirectly coupled. For example, the target antigen can be comprised in a labeling agent, wherein the labeling agent further comprises a support, and wherein (i) the target antigen is coupled to the support via a ligand, and (ii) the first reporter oligonucleotide is coupled to the support. In some embodiments, the target antigen is covalently conjugated to the ligand.

In some embodiments, the target antigen comprises a target MHC molecule complex, wherein the target MHC molecule complex comprises an MHC molecule bound to a target antigenic molecule. The target MHC molecule complex can be further coupled to a support via a ligand, and the first reporter oligonucleotide can be coupled to the support. In some embodiments, the target antigenic molecule is an antigenic peptide, a lipid, or a small molecule. In some embodiments, the target antigen comprises a plurality of target MHC molecule complexes, optionally wherein the target MHC molecule complexes are covalently conjugated to the ligand. In some embodiments, wherein the target antigen comprises a plurality of target MHC molecule complexes. For example, in some embodiments, the target antigen comprises four target MHC molecule complexes

The support can be selected from avidin, streptavidin, deglycosylated avidin (e.g., NeutrAvidin™), traptavidin, tamavidin, xenavidin, bradavidin, AVR2 (avidin related protein 2), AVR4 (avidin related protein 4), and variants, mutants, derivatives, and homologs of any thereof. In some embodiments, the ligand comprises biotin. In some embodiments, the labeling agent or target MHC molecule complex further comprises a fluorescent agent.

In some embodiments, the ABM is expressed by a cell comprised within the biological tissue. The cell can be, for example, an immune cell. Exemplary immune cells include, without limitation, B-cells and T-cells. In some embodiments, the B cell is a plasmablast, a plasma cell, a memory B cell, a regulatory B cell, or a lymphoplasmacytoid cell. In some embodiments, the ABM is an antibody or fragment thereof, or a T-cell receptor or fragment thereof. In some embodiments, the antibody is a secreted antibody. In some embodiments, the secreted antibody is in proximity to the cell. The methods can further include in step (a) contacting the biological sample with a plurality of labeling agents, wherein the labeling agents are configured to bind or otherwise couple to one or more surface features of the cell.

In some embodiments, the biological sample is disposed (e.g., mounted) on the array. In some embodiments, the biological sample is disposed on a first substrate and the array is attached to a second substrate, and wherein the method further includes aligning the first substrate with the second substrate such that at least a portion of the biological sample is aligned with at least a portion of the array.

In some embodiments, the biological sample is disposed on a first substrate during (b) and the array is attached to a second substrate, and wherein the method includes, following (b): mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the biological sample comprising the ABM is aligned with a portion of the array of capture probes and within a threshold distance of the array of capture probes, and such that the portion of the biological sample and the capture probes contact the reagent medium. In some embodiments, the permeabilization agent releases the nucleic acid comprising a sequence encoding at least a portion of the ABM or a reverse complement thereof from the biological sample.

In some embodiments, the methods further include prior to the hybridizing in step (c), releasing the first reporter oligonucleotide and migrating the first reporter oligonucleotide to the array. In some embodiments, the nucleic acid comprising a sequence encoding at least a portion of the ABM or a reverse complement thereof is comprised in the biological sample, and the method further comprises releasing the nucleic acid comprising a sequence encoding at least a portion of the ABM or a reverse complement thereof from the biological sample, and migrating the nucleic acid comprising a sequence encoding at least a portion of the ABM or a reverse complement thereof to the array. The migrating can include passive migration or active migration. For example, active migration includes electrophoresis.

In some embodiments, the first reporter oligonucleotide and second reporter oligonucleotide further include one or more functional domains. In some embodiments, the first and second capture probes further include a cleavage domain, one or more functional domains, a unique molecular identifier, or a combination thereof.

In some embodiments, the first capture domain of the first capture probe and the second capture domain of the second capture probe are identical. In some embodiments, the capture domain of the first capture probe and the capture domain of the second capture probe are different. In some embodiments, the capture domain of the first capture probe is a defined non-homopolymeric sequence or a homopolymeric sequence. The homopolymeric sequence can include a polyT sequence and/or the non-homopolymeric sequence include a fixed sequence or a degenerate sequence. In some embodiments, the second capture domain of the second capture probe binds to the nucleic acid comprising a sequence encoding at least a portion of the ABM or a reverse complement thereof, wherein the ABM is an antibody or TCR. In some embodiments, the second capture domain of the second capture probe binds a poly(A) sequence of the nucleic acid comprising a sequence encoding at least a portion of the antibody or TCR or a reverse complement thereof. In some embodiments, the second capture domain of the second capture probe binds to the nucleic acid in a region encoding a constant region of the antibody or TCR.

In some embodiments, generating a second barcoded polynucleotide in step (e) comprises extending the capture probe using the nucleic acid comprising a sequence encoding at least a portion of the antibody or TCR or a reverse complement thereof as a template, thereby generating an extended capture probe; and optionally generating a complement of the extended capture probe. The methods can further include amplifying the second barcoded polynucleotide with a first primer that specifically binds to a functional sequence of the capture probe or reverse complement thereof and a second primer that binds to a nucleic acid sequence encoding a variable region of the antibody or TCR cell or reverse complement thereof, wherein the first primer and the second primer flank the spatial barcode of the second barcoded polynucleotide.

In some embodiments, the nucleic acid comprising a sequence encoding at least a portion of the antibody or TCR or a reverse complement thereof comprises a sequence encoding the variable region and constant region of the antibody or TCR. In some embodiments, generating a second barcoded polynucleotide in step (e) comprises extending the capture probe using the nucleic acid comprising the sequence encoding the variable region and constant region of the antibody or TCR as a template, thereby generating an extended capture probe; and amplifying the extended capture probe to provide a nucleic acid library. The methods can further include circularizing a member of the nucleic acid library to generate a circularized nucleic acid, and amplifying the circularized nucleic acid using a first primer and a second primer to generate a double-stranded member of the nucleic acid library lacking all, or a portion of, the sequence encoding a constant region of the antibody or TCR.

In some embodiments, said generating a second barcoded polynucleotide in step (e) comprises (i) contacting the biological sample with a first primer that hybridizes to the nucleic acid comprising a sequence encoding at least a portion of the ABM or a reverse complement thereof, wherein the first primer comprises a functional domain; (ii) extending the first primer using the nucleic acid comprising a sequence encoding at least a portion of the ABM or a reverse complement thereof as a template to generate an extension product; (iii) adding a polynucleotide sequence comprising at least three nucleotides to the 3′ end of the extension product; (iv) hybridizing a second primer to the polynucleotide sequence comprising at least three nucleotides of the extension product of (iii), wherein the second primer comprises a capture sequence; (v) extending the extension product using the second primer as a template, thereby incorporating a complement of the capture sequence into the extension product; (vi) hybridizing the complement of the capture sequence of the extension product to the second capture domain of the second capture probe; and (vii) extending the 3′ end of the extension product of (v) using the capture probe as a template, thereby generating an extended capture product.

In some embodiments, the first primer hybridizes to a region of the nucleic acid encoding a constant region of the ABM.

In some embodiments, said generating a first barcoded polynucleotide in step (d) comprises extending the first reporter oligonucleotide using the first capture probe as a template, thereby providing an extended first reporter oligonucleotide. In some embodiments, the methods further include amplifying the extended first reporter oligonucleotide.

In some embodiments, the methods further include generating a third barcoded polynucleotide or a plurality of third barcoded polynucleotides comprising (i) the second reporter barcode sequence or reverse complement thereof, and (ii) the spatial barcode or reverse complement thereof, and optionally using the third barcoded polynucleotide or plurality of third barcoded polynucleotides to identify the ABM as having bound to the non-target antigen coupled to the second reporter oligonucleotide. In some embodiments, the determining and/or identifying in step (f) comprises determining sequences of the first barcoded polynucleotide and the second barcoded polynucleotide, and optionally determining a sequence of the third barcoded polynucleotide. In any of the foregoing, the determining can be performed by sequencing.

In some embodiments, the capture sequence (or capture domain) of a capture probe on the array is configured to couple to the capture handle sequence of the reporter oligonucleotide by complementarity base pairing. In some embodiments, the capture sequence is configured to couple to an mRNA analyte, and wherein the capture sequence configured to couple to the mRNA analyte includes a polyT sequence.

In some embodiments, the capture sequence is configured to couple to the mRNA analyte, and wherein the capture sequence configured to couple to the mRNA analyte comprises a targeted priming sequence, optionally wherein the targeted priming sequence targets an antibody or BCR region of the mRNA analyte, e.g., a constant sequence of said antibody or BCR region of the mRNA analyte. In some embodiments, a capture probe of the array comprises a capture sequence configured to couple to the capture handle sequence, and wherein a second nucleic acid barcode molecule of the plurality of nucleic acid barcode molecules further comprises a capture sequence configured to couple to non-templated nucleotides appended to a cDNA reverse transcribed from an mRNA analyte. In some embodiments, the mRNA analyte is reverse transcribed to the cDNA utilizing a primer comprising a polyT sequence. In some embodiments, the non-templated nucleotides appended to the cDNA comprise a cytosine. In some embodiments, the capture sequence configured to couple to the cDNA comprise a guanine. In some embodiments, the coupling of the capture sequence to the non-templated cytosine extends reverse transcription of the cDNA into the second nucleic acid barcode to generate the second barcoded nucleic molecule. In some embodiments, the second nucleic acid barcode molecule further comprises a template switch oligonucleotide (TSO).

In some embodiments, the method includes identifying the ABM based on the determined sequence of the second barcoded polynucleotide. The determined sequence can include a nucleotide sequence. In some embodiments, the determined sequence can include an amino acid sequence encoded by the nucleotide sequence.

In some embodiments, the binding affinity of the ABM to the target antigen is assessed based on the determined sequence of the first barcoded nucleic acid molecule.

In some embodiments, the nucleic acid comprising a sequence encoding at least a portion of the ABM or a reverse complement thereof is RNA or DNA. In some embodiments, the RNA is mRNA and the DNA is genomic DNA or cDNA.

The methods further can further involve fixing the biological sample. Fixing the biological sample can include the use of a fixative selected from the group consisting of: ethanol, methanol, acetone, formaldehyde, paraformaldehyde-Triton, glutaraldehyde, and combinations thereof.

In some embodiments, the methods further comprise staining the biological sample. The staining can include use of eosin and/or hematoxylin. In some embodiments, the staining comprises the use of a detectable label selected from the group consisting of a radioisotope, a fluorophore, a chemiluminescent compound, a bioluminescent compound, or a combination thereof.

In some embodiments, the method further includes imaging the biological sample. The imaging can involve one or more of expansion microscopy, bright field microscopy, dark field microscopy, phase contrast microscopy, electron microscopy, fluorescence microscopy, reflection microscopy, interference microscopy and confocal microscopy.

The methods can further include a step of permeabilizing the biological sample. The permeabilizing can involve the use of an organic solvent, a detergent, an enzyme, or a combination thereof. In some embodiments, the permeabilizing comprises the use of an endopeptidase, wherein the endopeptidase is pepsin or proteinase K, a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof.

### Target and Non-Target Antigens

A target antigen may be any antigen of interest. As used herein, an “antigen” is not limited to proteins, fats, and/or sugars that is foreign to the subject but may include self-antigens, e.g., amyloid or tau protein. The target antigen may be any antigen for which the identification and/or characterization of antigen-binding molecule such as an antibody or antigen-binding fragment thereof, or T-cell receptor (TCR) or antigen-binding fragment thereof, capable of binding or having an affinity thereto is desirable. In some embodiments, the target antigen is a peptide. In some embodiments, the target antigen may be a protein or peptide as expressed by a cell, e.g., full-length target antigen that may or may not include its leader sequence and may or may not have undergone a similar cell processing step.

In some embodiments, the target antigen is associated with a disease or disorder, such as, but not limited to, cancer, an autoimmune disease, a neurodegenerative disease, an infectious disease, or an inflammatory disease. For example, the target antigen can be a cancer antigen or a coronavirus derived antigen.

The target antigen may be an antigen associated with an infectious agent, such as a viral, bacterial, parasitic, protozoal or prion agent. Exemplary infectious agents include a human immunodeficiency virus (HIV), an influenza virus, a coronavirus, a retrovirus, a rhinovirus, or a sarcoma virus. The viral agent may be severe acute respiratory syndrome coronavirus 1 (SARS-CoV-1), a SARS-CoV-2, a Middle East respiratory syndrome coronavirus (MERS-CoV), influenza, respiratory syncytial virus, or Ebola virus. Other exemplary viral agents include corona virus spike (S) protein, an influenza hemagglutinin protein, an HIV envelope protein or any other a viral glycoprotein.

In some embodiments, the target antigen is associated with a tumor or a cancer. Exemplary cancer antigens include, without limitation, epidermal growth factor receptor (EGFR), CD38, platelet-derived growth factor receptor (PDGFR) alpha, insulin growth factor receptor (IGFR), CD20, CD19, CD47, or human epidermal growth factor receptor 2 (HER2). In addition, the target antigen may be an immune checkpoint molecule that may or may not be associated with tumors or cancers (e.g., CD38, PD-1, CTLA-4, TIGIT, LAG-3, VISTA, TIM-3), or it may be a cytokine, a GPCR, a cell-based co-stimulatory molecule, a cell-based co-inhibitory molecule, an ion channel, or a growth factor. Further still, the target antigen may be associated with a degenerative condition or disease (e.g., an amyloid protein).

In some embodiments, the target antigen is derived from a biological sample from a subject previously exposed to the target antigen or suspected of having been exposed to the target antigen, or an evolutionarily related antigen. The evolutionarily related antigen can belong to the same family, the same sub-family, the same genus. The evolutionarily related antigen can be a variant of the target antigen and be the same species. The evolutionarily related antigen can have at least 75% identity as the target antigen. The evolutionarily related antigen can be a functional variant. For example, in cases where the target antigen is a disease-associated antigen, the subject can be previously known or suspected of having the disease. In some embodiments, the subject can have been immunized with the target antigen. In some embodiments, the subject can have recovered from the disease.

In some embodiments, the non-target antigen may be any antigen to which the ABMs or fragments thereof would not be expected to bind. In some embodiments, the non-target antigen does not significantly bind the ABMs or fragments thereof.

In some embodiments, the non-target antigen has been selected such that it is not expected to bind the ABM or antigen-binding fragment thereof. In some embodiments, the non-target antigen has been selected such that it is not expected to significantly bind the ABM or antigen-binding fragment thereof. In some embodiments, the non-target antigen is an off-target antigen for which binding to an ABM or antigen-binding fragment is undesirable. By way of example, the non-target antigen may be any antigen for which a subject, e.g., a human would not be expected to develop an immune response to or to have antibodies with a specificity for. Such a non-target antigen may be an antigen endogenous to and abundantly expressed in a subject, e.g., a human subject, e.g., human serum albumin. Other suitable non-target antigens (which can be negative control antigens) are further described herein.

The number of control antigens can vary dependent on specific experimental parameters and can be about 1 to about 100, for example can be 2, 3, 4, 5, 6, 7, 8, 9, or 10. In some embodiments, the plurality of antigens including from about 5 to about 100, from about 10 to about 50, from about 15 to about 70, from about 20 to about 80, from about 30 to about 90, or from about 40 to about 100 non-target antigens. In some embodiments, the plurality of antigens including from about 5 to about 50, from about 10 to about 30, from about 20 to about 40, from about 40 to about 100, from about 15 to about 60, from about 20 to about 80, from about 50 to about 100, from about 30 to about 80, from about 1 to about 10, or from about 5 to about 20 non-target antigens.

Generally, two conceptually different classes of control antigens can be included: “in-line controls” and “process-reassurance controls.” In-line control antigens can be sample-dependent, which subsequently can aid in data analysis and successful interpretation of data. Alternatively or in addition, “process-reassurance control” antigens can also be included. This type of control antigens (e.g., positive control antigens) can be dependent on sample antigen specificities in order to confirm that experiments protocols work properly in experimenters' hands. Inclusion of process-reassurance controls can be optional.

In some embodiments, negative control antigens can include a reporter oligonucleotide associated with any one or more of: a detectable label, a support, and a ligand with binding affinity for a binding region of the support. Exemplary detectable labels (e.g., fluorophores) are further disclosed herein. Exemplary supports (e.g., streptavidin, avidin) and their ligands (e.g., biotin) are also further disclosed herein. For example, a negative control antigen can be or include a complex comprising a detectable label, support, and/or ligand with binding affinity for a binding region of the support. For example, a negative control antigen can be or can include a biotin-saturated streptavidin comprising a detectable label and/or a reporter oligonucleotide. Such negative control antigens can be used to distinguish antibodies that specifically bind a target antigen from antibodies that non-specifically bind to any one or more of the detectable label (e.g., fluorophore), support (e.g., streptavidin, avidin), and ligand (e.g., biotin).

In some embodiments of the methods disclosed herein, negative control antigens can include a streptavidin molecule saturated with biotin, wherein the streptavidin is coupled to a reporter oligonucleotide. In some embodiments, negative control antigens can include (i) biotinylated human serum albumin complexed with a streptavidin, wherein the streptavidin is coupled to a reporter oligonucleotide.

### Biological Samples

In principle, there are no particular restrictions in regard to the types of biological samples suitable for use in the methods described herein. For example, the biological sample may be obtained by biopsy or core biopsy.

A sample can be derived from any useful source including any subject, such as a human subject. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, can be obtained for analysis as described herein. For example, a first sample can be obtained from a subject at a first time and a second sample can be obtained from the subject at a second time later than the first time. The first time can be before a subject undergoes a treatment regimen or procedure (e.g., to address a disease or condition), and the second time can be during or after the subject undergoes the treatment regimen or procedure. In another example, a first sample can be obtained from a first bodily location or system of a subject (e.g., using a first collection technique) and a second sample can be obtained from a second bodily location or system of the subject (e.g., using a second collection technique), which second bodily location or system can be different than the first bodily location or system. In another example, multiple samples can be obtained from a subject at a same time from the same or different bodily locations. Different samples, such as different samples collected from different bodily locations of a same subject, at different times, from multiple different subjects, and/or using different collection techniques, can undergo the same or different processing (e.g., as described herein). For example, a first sample can undergo a first processing protocol and a second sample can undergo a second processing protocol.

A sample can be a biological sample, such as a tissue sample. The tissue sample can be obtained from any suitable tissue, e.g., spleen, tonsils, lung, lymph node, bone, brain, etc. The sample can be a lymph node sample. The sample can be a lymphoid tissue (e.g. tonsil, mucosal-associated lymphoid tissue) sample. In some embodiments, the biological sample can be a skin sample.

A sample can include one or more analytes or analyte carriers, such as one or more cells and/or cellular constituents, such as one or more cell nuclei. For example, a sample can include a plurality of cells and/or cellular constituents. Components (e.g., cells or cellular constituents, such as cell nuclei) of a sample can be of a single type or a plurality of different types. For example, cells of a sample can include one or more immune cells. In some embodiments, the biological sample includes one or more cells, such as immune cells. For example, the biological sample can include B-cells and/or T-cells.

A sample may undergo one or more processes in preparation for analysis including, but not limited to, isolation, agitation, fixation, permeabilization, heating, and/or other processes.

In some embodiments, the biological sample is from a vertebrate subject. For example, the vertebrate subject can be a mammalian subject, such as a human. In some embodiments, the biological sample is a tissue sample. Non-limiting examples of tissue samples include a fixed tissue sample, such as a formalin-fixed paraffin embedded tissue sample, a paraformaldehyde fixed tissue sample, a methanol fixed tissue sample, or an acetone fixed tissue sample. In some embodiments, the tissue sample is a fresh frozen tissue sample.

In some embodiments, the biological sample is a tissue section. The tissue section can be a fixed tissue section, such as a formalin-fixed paraffin embedded tissue section, a paraformaldehyde fixed tissue section, a methanol fixed tissue section, or an acetone fixed tissue section.

In some embodiments, the biological sample is a diseased tissue sample and/or a tissue sample derived from a subject having a disease or disorder. The disease or disorder can be cancer, an autoimmune disease, a neurodegenerative disease, an infectious disease, or an inflammatory disease. Biological samples can include one or more diseased cells. A diseased cell can have altered metabolic properties, gene expression, protein expression, and/or morphologic features. Cancer cells can be derived from solid tumors, hematological malignancies, cell lines, or obtained as circulating tumor cells.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, the biological sample, e.g., the tissue sample, is fixed in a fixative including alcohol, for example methanol. In some embodiments, instead of methanol, acetone, or an acetone-methanol mixture can be used. In some embodiments, the fixation is performed after sectioning

In some embodiments, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, acid and a soluble organic compound that preserves morphology and bio-molecules. It is a two-reagent fixative system in which tissue is firstly fixed in a solution containing methanol and acetic acid then stabilized in a solution containing ethanol. See, Ergin B. et al., J Proteome Res. 2010 Oct. 1; 9(10):5188-96; Kap M. et al., PLoS One.; 6(11):e27704 (2011); and Mathieson W. et al., Am J Clin Pathol.; 146(1):25-40 (2016), each of which are hereby incorporated by reference in their entirety, for a description and evaluation of PAXgene for tissue fixation. Thus, in some embodiments, when the biological sample, e.g., the tissue sample, is fixed in a fixative including alcohol, the fixative is PAXgene.

In some embodiments, the tissue sample can be derived from skin, brain, breast, lung, liver, kidney, prostate, tonsil, thymus, testes, bone, lymph node, ovary, eye, heart, femur, tibia, or spleen.

In some embodiments, the biological sample (e.g., tissue sample) is a tissue microarray (TMA). A tissue microarray contains multiple representative tissue samples—which can be from different tissues or organisms—assembled on a single histologic slide. The TMA can therefore allow for high throughput analysis of multiple specimens at the same time. Tissue microarrays are paraffin blocks produced by extracting cylindrical tissue cores from different paraffin donor blocks and re-embedding these into a single recipient (microarray) block at defined array coordinates.

### Labeling Agents & Multiplexing Methods

in some embodiments of the disclosure, the methods described herein are performed in multiplex format. For example, a plurality of target antigens can be used to identify and/or characterize ABMs within a biological sample that bind thereto.

Accordingly, in some embodiments, the present disclosure provides methods and systems for multiplexing, and otherwise increasing throughput of samples for analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cells or cell features (e.g., within a tissue sample) can be used to characterize cells and/or cell features. In some instances, cell features include cell surface features. Cell surface features can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features can include intracellular analytes, such as proteins, protein modifications (e.g., phosphorylation status or other post-translational modifications), nuclear proteins, nuclear membrane proteins, or any combination thereof.

A labelling agent can include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide can include a barcode sequence that permits identification of the labelling agent. For example, a labelling agent that is specific to one type of cell feature (e.g., a first cell surface feature) can have a first reporter oligonucleotide coupled thereto, while a labelling agent that is specific to a different cell feature (e.g., a second cell surface feature) can have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969.

In a particular example, a library of potential cell feature labelling agents can be provided, where the respective cell feature labelling agents are associated with nucleic acid reporter molecules, such that a different reporter oligonucleotide sequence is associated with each labelling agent capable of binding to a specific cell feature. In other aspects, different members of the library can be characterized by the presence of a different oligonucleotide sequence label. For example, a target antigen capable of binding to a first ABM can have associated with it a first reporter oligonucleotide sequence, while another target antigen capable of binding to a second ABM can have a different reporter oligonucleotide sequence associated with it. The presence of the particular oligonucleotide sequence can be indicative of the presence of a particular feature which can recognize or bind a given target antigen.

In some embodiments, the reporter oligonucleotides can include a barcode sequence that permits identification of a pretreatment condition to which the biological sample (or subject from whom the biological sample is obtained) is subjected. In some embodiments, the pretreatment is performed prior to the step of contacting the B cells with the antigens.

For example, a reporter oligonucleotide can be linked to an antibody or an epitope binding fragment thereof, and labeling a cell can include subjecting the antibody-linked barcode molecule or the epitope binding fragment-linked barcode molecule to conditions suitable for binding the antibody to a molecule present on a surface of the cell. The binding affinity between the antibody or the epitope-binding fragment thereof and the molecule present on the surface can be within a desired range to ensure that the antibody or the epitope binding fragment thereof remains bound to the molecule. For example, the binding affinity can be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds can be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant can be less than about 10 μM. In some embodiments, the antibody or antigen-binding fragment thereof has a desired dissociation rate constant (k_{off}), such that the antibody or antigen binding fragment thereof remains bound to the target antigen or antigen fragment during various sample processing steps.

In another example, a reporter oligonucleotide can be coupled to a cell-penetrating peptide (CPP), and labeling cells can include delivering the CPP coupled reporter oligonucleotide into an analyte carrier. Labeling analyte carriers can include delivering the CPP conjugated oligonucleotide into a cell and/or cell bead by the cell-penetrating peptide. A CPP that can be used in the methods provided herein can include at least one non-functional cysteine residue, which can be either free or derivatized to form a disulfide link with an oligonucleotide that has been modified for such linkage. Non-limiting examples of CPPs that can be used in embodiments herein include penetratin, transportan, plsl, TAT(48-60), pVEC, MITS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The CPP can be an arginine-rich peptide transporter. The CPP can be Penetratin or the Tat peptide. In another example, a reporter oligonucleotide can be coupled to a fluorophore or dye, and labeling cells can include subjecting the fluorophore-linked barcode molecule to conditions suitable for binding the fluorophore to the surface of the cell. In some instances, fluorophores can interact strongly with lipid bilayers and labeling cells can include subjecting the fluorophore-linked barcode molecule to conditions such that the fluorophore binds to or is inserted into a membrane of the cell. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, for a description of organic fluorophores.

A reporter oligonucleotide can be coupled to a lipophilic molecule, and labeling cells can include delivering the nucleic acid barcode molecule to a membrane of a cell or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and the cell or nuclear membrane can be such that the membrane retains the lipophilic molecule (e.g., and associated components, such as nucleic acid barcode molecules, thereof) during subsequent processing (e.g., partitioning, cell permeabilization, amplification, pooling, etc.). The reporter nucleotide can enter into the intracellular space and/or a cell nucleus. In some embodiments, a reporter oligonucleotide coupled to a lipophilic molecule will remain associated with and/or inserted into lipid membrane (as described herein) via the lipophilic molecule until lysis of the cell occurs, e.g., inside a partition. Exemplary embodiments of lipophilic molecules coupled to reporter oligonucleotides are described in PCT/US2018/064600.

A reporter oligonucleotide can be part of a nucleic acid molecule including any number of functional sequences, as described elsewhere herein, such as a target capture sequence, a random primer sequence, and the like, and coupled to another nucleic acid molecule that is, or is derived from, the analyte.

Prior to partitioning, the biological sample can be incubated with the library of labelling agents, that can be labelling agents to a broad panel of different cell features, e.g., receptors, proteins, etc., and which include their associated reporter oligonucleotides. Unbound labelling agents can be washed from the sample.

In other instances, e.g., to facilitate sample multiplexing, a labelling agent that is specific to a particular cell feature can have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent can interact with different cells in a tissue, allowing a particular report oligonucleotide to indicate a particular cell population in the tissue and cell feature.

In some instances, these reporter oligonucleotides can include nucleic acid barcode sequences that permit identification of the labelling agent which the reporter oligonucleotide is coupled to. The use of oligonucleotides as the reporter can provide advantages of being able to generate significant diversity in terms of sequence, while also being readily attachable to most biomolecules, e.g., antigens, etc., as well as being readily detected, e.g., using sequencing or array technologies.

Attachment (coupling) of the reporter oligonucleotides to the labelling agents can be achieved through any of a variety of direct or indirect, covalent or non-covalent associations or attachments. For example, oligonucleotides can be covalently attached to a portion of a labelling agent (such a protein, e.g., an antibody or antibody fragment), e.g., via a linker, using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715. Likewise, protein and peptide biotinylation techniques have been developed and are readily available. See, e.g., U.S. Pat. No. 6,265,552. Furthermore, click reaction chemistry such as 5′ Azide oligos and Alkyne-NHS for click chemistry, 4′-Amino oligos for HyNic-4B chemistry, a Methyltetrazine-PEG5-NHS Ester reaction, a TCO-PEG4-NHS Ester reaction, strain-promoted alkyne-azide cycloaddition (SPAAC), or the like, can be used to couple reporter oligonucleotides to labelling agents. Commercially available kits, such as those from Thunderlink and Abcam, and techniques common in the art can be used to couple reporter oligonucleotides to labelling agents as appropriate. In another example, a labelling agent is indirectly (e.g., via hybridization) coupled to a reporter oligonucleotide including a barcode sequence that identifies the label agent. For instance, the labelling agent can be directly coupled (e.g., covalently bound) to a hybridization oligonucleotide that includes a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide can be attached to the labeling agent through a labile bond (e.g., chemically labile, photolabile, thermally labile, etc.) as generally described for releasing molecules from supports elsewhere herein. In some instances, the reporter oligonucleotides described herein can include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent (e.g., an antigen, an antigen fragment, an antibody, an antibody fragment) is presented as a monomer. In some cases, the labelling agent is presented as a multimer. In some cases, a labelling agent (e.g., an antigen, an antigen fragment, an antibody, an antibody fragment) is presented as a dimer. In some cases, a labelling agent (e.g., an antigen, an antigen fragment, an antibody, an antibody fragment) is presented as a trimer. In some cases, a labelling agent (e.g., an antigen, an antigen fragment, an antibody, an antibody fragment) is presented as a tetramer. In some cases, a labelling agent (e.g., an antigen, an antigen fragment, an antibody, an antibody fragment) is presented as a pentamer. In some cases, a labelling agent (e.g., an antigen, an antigen fragment, an antibody, an antibody fragment) is presented as a hexamer. In some cases, a labelling agent (e.g., an antigen, an antigen fragment, an antibody, an antibody fragment) is presented as a heptamer. In some cases, a labelling agent (e.g., an antigen, an antigen fragment, an antibody, an antibody fragment) is presented as an octamer. In some cases, a labelling agent (e.g., an antigen, an antigen fragment, an antibody, an antibody fragment) is presented as a nonamer. In some cases, a labelling agent (e.g., an antigen, an antigen fragment, an antibody, an antibody fragment) is presented as a decamer. In some cases, a labelling agent (e.g., an antigen, an antigen fragment, an antibody, an antibody fragment) is presented as a 10+-mer.

In some cases, the labelling agent can include a reporter oligonucleotide and a label (e.g., detectable label). A label (e.g., detectable label) can be a fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide can be allowed to hybridize to the reporter oligonucleotide.

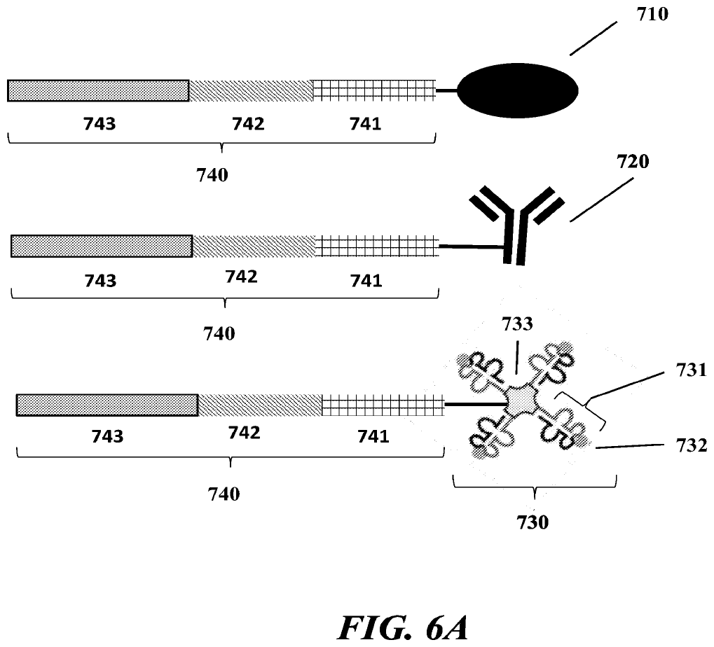

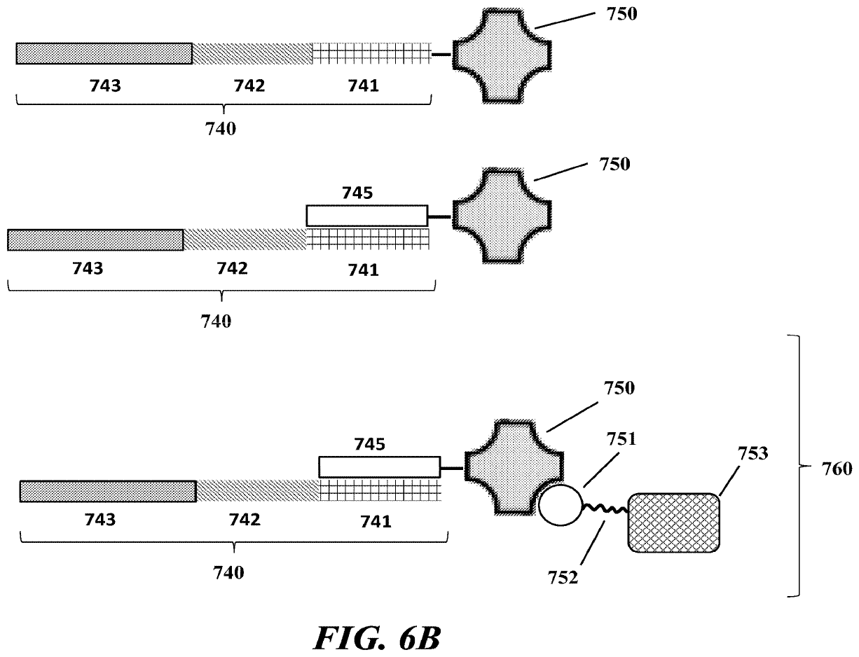

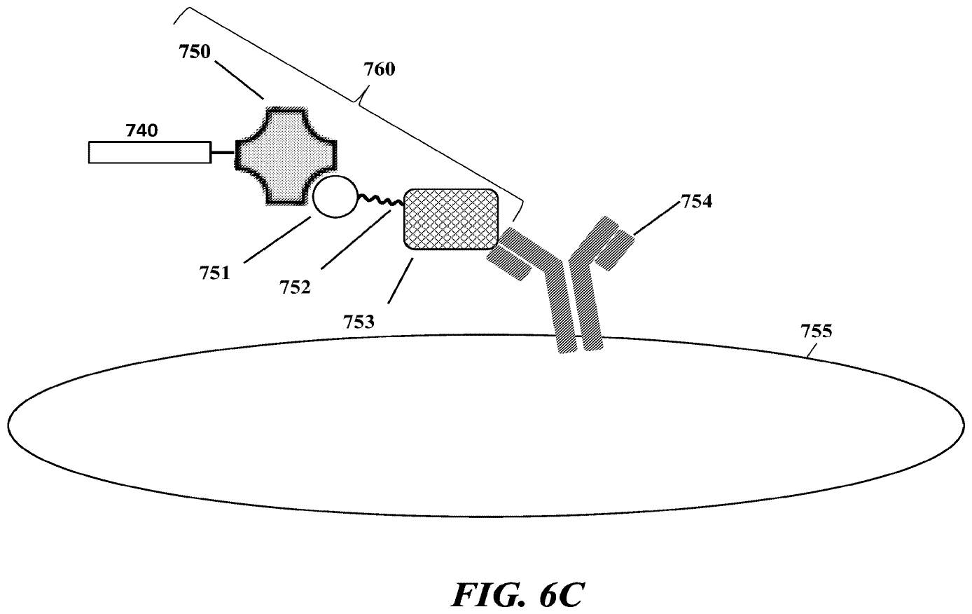

FIG. 6A describes exemplary labelling agents (710, 720, 730) including reporter oligonucleotides (740) attached thereto. Labelling agent 710 (e.g., any of the labelling agents described herein) is attached (either directly, e.g., covalently attached, or indirectly) to reporter oligonucleotide 740. Reporter oligonucleotide 740 can include barcode sequence 742 that identifies labelling agent 710. Reporter oligonucleotide 740 can also include one or more functional sequences 741 that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 6A, in some instances, reporter oligonucleotide 740 conjugated to a labelling agent (e.g., 710, 720, 730) includes a functional sequence 741, a reporter barcode sequence 742 that identifies the labelling agent (e.g., 710, 720, 730), and reporter capture handle 743. Reporter capture handle sequence 743 can be configured to hybridize to a complementary sequence, such as a complementary sequence present on a capture probe on a spatial array, such as those described elsewhere herein. In some instances, reporter oligonucleotide 740 includes one or more additional functional sequences, such as those described above.

In some instances, the labelling agent 710 is a protein or polypeptide (e.g., an antigen or prospective antigen) including reporter oligonucleotide 740. Reporter oligonucleotide 740 includes reporter barcode sequence 742 that identifies polypeptide 710 and can be used to infer the presence of an analyte, e.g., a binding partner of polypeptide 710 (i.e., a molecule or compound to which polypeptide 710 can bind, such as an ABM). In some instances, the labelling agent 710 is a lipophilic moiety (e.g., cholesterol) including reporter oligonucleotide 740, where the lipophilic moiety is selected such that labelling agent 710 integrates into a membrane of a cell or nucleus. In some instances, the labelling agent is an antibody 720 (or an epitope binding fragment thereof) including reporter oligonucleotide 740. Reporter oligonucleotide 740 includes reporter barcode sequence 742 that identifies antibody 720 and can be used to infer the presence of, e.g., a target of antibody 720 (i.e., a molecule or compound to which antibody 720 binds).

In other embodiments, labelling agent 730 includes an MHC molecule 731 including peptide 732 and reporter oligonucleotide 740 that identifies peptide 732. In some instances, the MHC molecule is coupled to a support 733. In some instances, support 733 can be a polypeptide, such as streptavidin, or a polysaccharide, such as dextran. In some instances, reporter oligonucleotide 740 can be directly or indirectly coupled to MHC labelling agent 730 in any suitable manner. For example, reporter oligonucleotide 740 can be coupled to MHC molecule 731, support 733, or peptide 732. In some embodiments, labelling agent 730 includes a plurality of MHC molecules, (e.g. is an MHC multimer, which can be coupled to a support (e.g., 733)). In some embodiments, the reporter oligonucleotide and MHC molecule are attached to the polypeptide or polysaccharide support. In some embodiments, the reporter oligonucleotide and MHC molecule are attached to the detectable label of the support. In some embodiments, the reporter oligonucleotide and an antigen (e.g., protein, polypeptide) are attached to the polypeptide or polysaccharide of the support. In some embodiments, the reporter oligonucleotide and an antigen (e.g., protein, polypeptide) are attached to the detectable label of the support. There are many possible configurations of Class I and/or Class II MHC multimers that can be utilized with the methods and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969.

Referring to FIG. 6B, in some instances, reporter oligonucleotide 740 is conjugated to a support 750 that can be used to complex with or bind to an antigen (e.g., an antigen of interest or a non-target antigen). Reporter oligonucleotide 740 includes a functional sequence 741, a reporter barcode sequence 742 that identifies the antigen of interest, and reporter capture handle 743. Reporter capture handle sequence 743 can be configured to hybridize to a complementary sequence, such as a complementary sequence present on a capture domain of a capture probe on a spatial array (not shown), such as those described elsewhere herein. In some instances, reporter oligonucleotide 740 includes one or more additional functional sequences, such as those described above. In one other embodiment, support 750 comprises an anchor sequence 745 that is complementary to functional sequence 741. The reporter oligonucleotide 740 may be attached to support 750 via hybridization to anchor sequence 745. The anchor sequence 745 may further comprise (or may be) a functional sequence (similar to or equivalent to functional sequence 741) as described herein. In some embodiments, the anchor sequence 745 does not comprise a functional sequence. In some embodiments, reporter oligonucleotide 740 includes a functional sequence (not shown). A support 750 may comprise a binding region that can be used to complex with (or bind to) an antigen of interest. In one embodiment, the antigen of interest comprises a ligand that can be bound by the binding region of support 750.

Referring to FIG. 6B again, labeling agents for antigen receptor analysis are provided. In one embodiment, labelling agent 760 comprises a support 750 that includes an antigen of interest 753 and reporter oligonucleotide 740 that identifies the antigen 753 (e.g., via reporter barcode sequence 742). In some embodiments, the support 750 is coupled to, complexed with, or bound to a ligand 751. In some embodiments, support 750 can be a polypeptide. In some embodiments, the polypeptide can be streptavidin. In some embodiments, the polypeptide can be avidin. In some embodiments, support 750 can be a polysaccharide. In some embodiments, the polysaccharide can be dextran. In some embodiments, the polysaccharide can be a dextran. The ligand 751 can be a molecule with affinity for the binding region of the support 750. For example, the ligand 751 may be biotin and the support 750 may be a streptavidin support. In other embodiments, the ligand 751 is coupled to or conjugated to antigen 753 via a linker 752. Accordingly, in some embodiments of the disclosure, the partitioned cells are contacted with one or more biotinylated antigens. In some embodiments, the antigens can include Avitag biotinylation site and/or a His tag. Protein biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, and U.S. Pat. No. 6,265,552. In some embodiments, the partitioned cells are contacted with full-length coronavirus spike proteins comprising a trimerization domain. In some embodiments, reporter oligonucleotide 740 can be directly or indirectly coupled to labelling agent 760 in any suitable manner. For example, reporter oligonucleotide 740 can be coupled to the antigen 753, support 750, anchor sequence 745, or ligand 751.

Referring to FIG. 6C, a labelled cell 755 (e.g., within a tissue sample) comprising an antigen receptor of interest 754 is depicted. The labelling agent 760 can be contacted with a plurality of cells comprising antigen receptors of interest. In one example, an antigen receptor of interest 754 is bound by or labeled with the labelling agent 760 via an interaction between the antigen receptor of interest 754 and the antigen 753. Further downstream processing of the labelled sample 755 can be performed in according to the spatial analysis methods and system described herein.

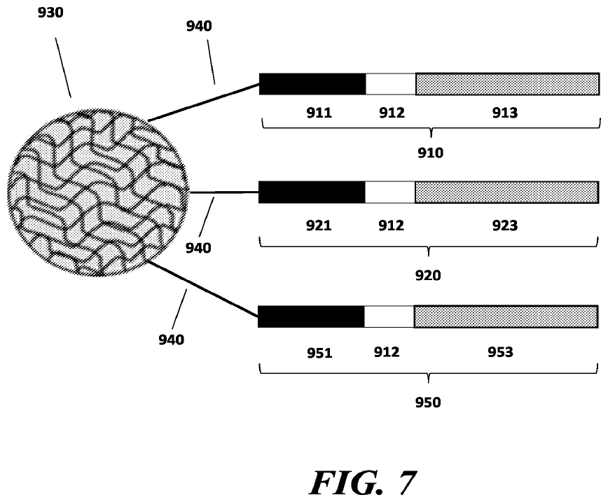

Exemplary capture probes attached to a support (e.g., a feature on a spatial array) are shown in FIG. 7. In some embodiments, analysis of multiple analytes (e.g., RNA and one or more analytes using labelling agents described herein) can include use of barcoded molecules as generally depicted in FIG. 7. In some embodiments, capture probes 910 and 920 are attached to support 930 via a releasable linkage 940 (e.g., including a labile bond) as described elsewhere herein. Capture probe 910 can include functional sequence 911, barcode sequence 912 and capture sequence (also referred to as “capture domain”) 913. Capture probe 920 can include adapter sequence 921, barcode sequence 912, and adapter sequence 923, wherein adapter sequence 923 includes a different sequence than adapter sequence 913. In some instances, adapter 911 and adapter 921 include the same sequence. In some instances, adapter 911 and adapter 921 include different sequences. Although support 930 is shown including capture probes 910 and 920, any suitable number of capture probes including common barcode sequence 912 are contemplated herein. For example, in some embodiments, support 930 further includes capture probe 950. Capture probe 950 can include adapter sequence 951, barcode sequence 912 and adapter sequence 953, wherein adapter sequence 953 includes a different sequence than adapter sequence 913 and 923. In some instances, capture probes (e.g., 910, 920, 950) include one or more additional functional sequences, such as a UMI or other sequences described herein. The capture probes 910, 920 or 950 can interact with analytes as described elsewhere herein, for example, as depicted in FIGS. 5, 8, 12A-12B, 20A and 22.

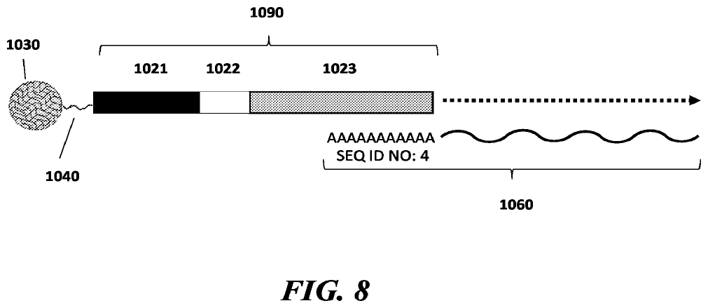

In some instances, analysis of an analyte (e.g. a nucleic acid, a polypeptide, a carbohydrate, a lipid, etc.) includes a workflow as generally depicted in FIG. 8. Referring to FIG. 8, in some embodiments, capture probe 1090 includes capture domain 1023 complementary to a sequence of RNA molecule 1060 from a biological sample, such as a tissue. In some instances, capture domain 1023 includes a sequence specific for an RNA molecule. Capture domain 1023 can include a known or targeted sequence or a random sequence. In some instances, a nucleic acid extension reaction can be performed, thereby generating a barcoded nucleic acid product including capture sequence 1023, the functional sequence 1021, UMI and/or barcode sequence 1022, any other functional sequence, and a sequence corresponding to the RNA molecule 1060.

In another example, capture sequence 1023 can be complementary to an overhang sequence or an adapter sequence that has been appended to an analyte. In one embodiment, capture sequence 1023 is complementary to a sequence that has been appended to a nucleic acid molecule derived from an analyte of interest. In another embodiment, the nucleic acid molecule is a cDNA molecule generated in a reverse transcription reaction using an RNA analyte (e.g., an mRNA analyte) of interest. In an additional embodiment, capture sequence 1023 is complementary to a sequence that has been appended to the cDNA molecule generated from the mRNA analyte of interest. For example, a complementary molecule be cDNA generated in a reverse transcription reaction. In some instances, an additional sequence can be appended to complementary molecule. For example, the reverse transcriptase enzyme can be selected such that several non-templated bases (e.g., a poly-C sequence) are appended to the cDNA. In another example, a terminal transferase can also be used to append the additional sequence. The capture probe includes a sequence complementary to the non-templated bases, and the reverse transcriptase performs a template switching reaction.

In some instances, capture sequence 1023 includes a sequence complementary to a region of an immune molecule, such as the constant region of a BCR sequence. Capture sequence 1023 is hybridized to nucleic acid molecule 1060 and a complementary molecule is generated. For example, the complementary molecule can be generated in a reverse transcription reaction generating a barcoded extension product including spatial barcode sequence 1022 (or a reverse complement thereof) and a sequence of complementary molecule 1070 (or a portion thereof). Additional methods and compositions suitable for barcoding cDNA generated from mRNA transcripts including those encoding V(D)J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, and U.S. Patent Publication No. 2019/0367969 and herein.

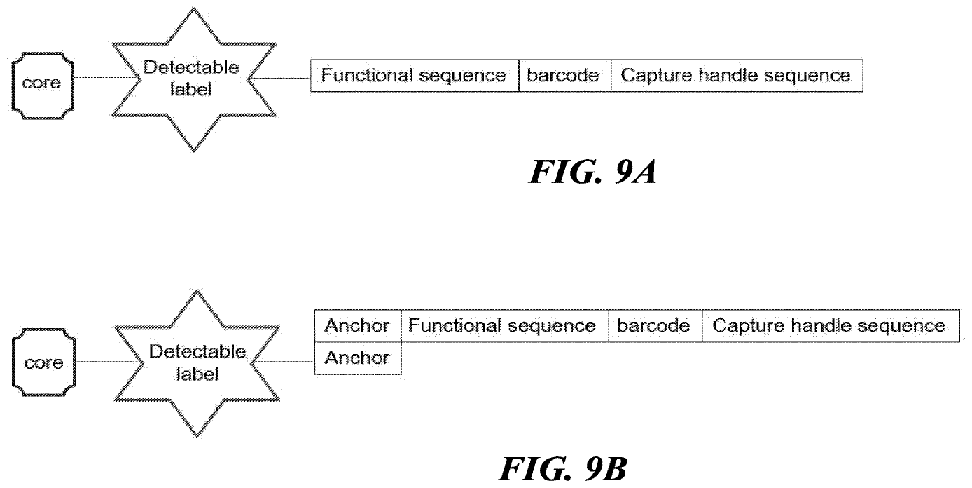

One aspect of the present disclosure relates to labelling compositions that can be useful for, (see, e.g., FIG. 9A), identification and/or characterization of ABMs, e.g., antibodies, BCRs, TCRs, and TCR-like antibodies. In some embodiments, the labelling compositions include a detectable label (e.g., a fluorophore (“FL”), a magnetic particle, or a mass tag; a core support (“core”) attached to the detectable label; and a nucleic acid molecule attached to the detectable label, wherein the nucleic acid molecule includes: (a) a reporter oligonucleotide including (i) a reporter barcode sequence and (ii) a capture handle sequence, or (b) an anchor nucleic acid molecule configured to directly or indirectly attach to the reporter oligonucleotide of (a).

Non-limiting exemplary embodiments of the labelling compositions disclosed herein can include one or more of the following features. In some embodiments, the anchor nucleic acid molecule includes a sequence configured to attach, by complementarity base pairing, to an anchoring sequence present in the reporter oligonucleotide (see, e.g., FIG. 9B).

In some embodiments, the labelling compositions of the disclosure include an anchor nucleic acid that is hybridized to the anchoring sequence of the reporter oligonucleotide.

In some embodiments, the reporter oligonucleotide further includes one or more functional sequences useful in the processing of the reporter oligonucleotide and/or barcoded nucleic acid molecules comprising a sequence of the reporter oligonucleotide. Suitable functional sequences include, but are not limited to, adapter sequences, primer sequences, primer binding sequences, unique molecular identifiers (UMIs), and hybridization or probing sequences, e.g., for identification of presence of the sequences or for pulling down reporter oligonucleotide and barcoded nucleic acids, or any of a number of other potential functional sequences.

In some embodiments, the detectable label includes a fluorophore, a magnetic particle, or a mass tag. In some embodiments, the detectable label includes a fluorophore molecule. In some embodiments, the fluorophore is or includes one or more of the following: phycoerythrin (PE), allophycocyanin (APC), Alexa Fluor 405, Pacific Blue, Alexa Fluor 488, Alexa Fluor 647, Alexa Fluor 700, DyLight 405, DyLight 550, DyLight 650, fluorescein isothiocyanate (FITC), peridinin chlorophyll protein (PerCP), StarBright Violet 440, StarBright Violet 515, StarBright 610, StarBright Violet 670, and StarBright Blue 700.

In some embodiments, the core support includes a biotin-binding agent. In some embodiments, the biotin-binding agent is or includes a biotin-binding protein. Suitable biotin-binding proteins include, but are not limited to streptavidin, avidin, deglycosylated avidin (e.g., NeutrAvidin™), traptavidin, tamavidin, xenavidin, bradavidin, AVR2 (avidin related protein 2), AVR4 (avidin related protein 4), and variants, mutants, derivatives, and homologs of any thereof. In some embodiments, the biotin-binding agent is or includes a biotin-binding protein selected from streptavidin, avidin, deglycosylated avidin (e.g., NeutrAvidin™) traptavidin, tamavidin, xenavidin, bradavidin, AVR2 (avidin related protein 2), and AVR4 (avidin related protein 4).

### MHC Molecule Complexes

The ABM identified or characterized in the methods, as provided herein, may be a TCR. The TCR is a molecule found on the surface of T cells that is generally responsible for recognizing fragments of antigen as peptides bound to major histocompatibility complex (MHC) molecules. The TCR is generally a heterodimer of two chains, each of which is a member of the immunoglobulin superfamily, possessing an N-terminal variable (V) domain, and a C terminal constant domain. In humans, in 95% of T cells the TCR consists of an alpha (α) and beta (β) chain, whereas in 5% of T cells the TCR consists of gamma and delta (γ/δ) chains. This ratio can change during ontogeny and in diseased states as well as in different species. In certain instances, TCR may be a human TCR, or a mouse TCR. In certain instances, the TCR may be a sheep, cow, rabbit or chicken TCR. In some instances, the TCR may be a scFv-like soluble TCR.

The ABM, identified or characterized by the methods provided herein, may be so identified or characterized by its having bound to, or having binding affinity for, a target MHC molecule complex. The target MHC molecule complex may include a target antigenic peptide, bound to an MHC molecule, to which binding by an ABM is desirable. The target antigenic peptide, bound to the MHC molecule of the target MHC molecule complex, may be a peptide or a peptide fragment of a target antigen associated with an infectious agent, such as a viral, bacterial, parasitic, protozoal or prion agent. In some instances, the target antigen, a peptide or peptide fragment of which may be the target antigenic peptide, may be an antigen associated a viral agent. In these instances, the viral agent may be an influenza virus, a coronavirus, a retrovirus, a rhinovirus, or a sarcoma virus. In other instances, the viral agent may be severe acute respiratory syndrome coronavirus 1 (SARS-CoV-1), a SARS-CoV-2, a Middle East respiratory syndrome coronavirus (MERS-CoV), or human immunodeficiency virus (HIV), influenza, respiratory syncytial virus, or Ebola virus. Examples of viral antigens that may be the target antigen, a peptide of which may be the target antigenic peptide bound to the MHC molecule of the target MHC molecule complex, include, but are not limited to, corona virus spike (S) protein, an influenza hemagglutinin protein, an HIV envelope protein or any other a viral glycoprotein. The target antigen, a peptide or peptide fragment of which may be the target antigenic peptide bound to the MHC molecule of the target MHC molecule complex, may alternatively be an antigen associated with a tumor or a cancer. Antigens associated with a tumor or cancer, include any of epidermal growth factor receptor (EGFR), CD38, platelet-derived growth factor receptor (PDGFR) alpha, insulin growth factor receptor (IGFR), CD20, CD19, CD47, ERBB2IP, TP53, KRAS, MAGEA1, LC3A2, KIAA0368, CADPS2, CTSB or human epidermal growth factor receptor 2 (HER2). Further, the target antigen, a peptide of which may be the target antigenic peptide bound to the MHC molecule of the target MHC molecule complex, may be an checkpoint molecule associated with tumors or cancers (e.g., CD38, PD-1, CTLA-4, TIGIT, LAG-3, VISTA, TIM-3), or it may be a cytokine, a GPCR, a cell-based co-stimulatory molecule, a cell-based co-inhibitory molecule, an ion channel, or a growth factor. Further still, the target antigen, a peptide of which may be the target antigenic peptide that binds the MHC molecule of the target MHC molecule complex, may be associated with a degenerative condition or disease. It will be understood that molecules other than antigenic peptides may be bound by the MHC molecule of the target MHC molecule complex, e.g., lipids or small molecule antigens.

The plurality of MHC molecule complexes may include: (i) a target MHC molecule complex and (ii) a non-target MHC molecule complex. The target MHC molecule complex may include a first MHC molecule and the non-target MHC molecule complex may include a second MHC molecule. The first MHC molecule of the target MHC molecule complex and/or the second MHC molecule of the non-target MHC molecule complex may be MHC class I or MHC class II molecules. In instances in which the first MHC molecule of the target MHC molecule complex and/or the second MHC molecule of the non-target MHC molecule complex is an MHC class I molecule, the MHC class I molecule may be a human MHC class I molecule. In instances in which the first MHC molecule of the target MHC molecule complex and/or the second MHC molecule of the non-target MHC molecule complex is a human MHC class I molecule, the human MHC class I molecule may be a human leukocyte antigen (HLA)-A, HLA-B, HLA-C, HLA-E, HLA-F or HLA-G molecule. In instances in which the first MHC molecule of the target MHC molecule complex and/or the second MHC molecule of the non-target MHC molecule complex is an HLA-A molecule, the HLA-A molecule may be of allele A*01:01, A*02:01, A*02:03, A*02:06, A*02:07, A*03:01, A*11:01, A*23:01, A*24:02, A*25:01, A*26:01, A*29:02, A*30:01, A*31:01, A*32:01, A*33:03, A*34:02, A*68:01, A*68:02, or A*74:01. In instances in which the first MHC molecule of the target MHC molecule complex and/or the second MHC molecule of the non-target MHC molecule complex is an HLA-B molecule, the HLA-B molecule may be of allele B*07:02, B*08:01, B*14:02, B*15:01, B*15:02, B*15:03, B*18:01, B*35:01, B*38:02, B*40:01, B*40:02, B*42:01, B*44:02, B*44:03, B*45:01, B*46:01, B*49:01, B*51:01, B*52:01, B*53:01, B*54:01, B*55:02, B*57:01 or B*58:01. In instances in which the first MHC molecule of the target MHC molecule complex and/or the second MHC molecule of the non-target MHC molecule complex is an HLA-C molecule, the HLA-C molecule may be of allele C*01:02, C*02:02, C*03:02, C*03:03, C*03:04, C*04:01, C*05:01, C*06:02, C*07:01, C*07:02, C*08:01, C*08:02, C*12:03, C*14:02, C*16:01, C*17:01 or C*18:01.

In instances in which the first MHC molecule of the target MHC molecule complex and/or the second MHC molecule of the non-target MHC molecule complex is an MHC class II molecule, the MHC class II molecule may be a human MHC class II molecule. In instances in which the first MHC molecule of the target MHC molecule complex and/or the second MHC molecule of the non-target MHC molecule complex is a human MHC class II molecule, the human MHC class II molecule may be a HLA-DP, HLA-DM, HLA-DOA, HLA-DOB, HLA-DQ or HLA-DR molecule. In instances in which the first MHC molecule of the target MHC molecule complex and/or the second MHC molecule of the non-target MHC molecule complex is a HLA-DR molecule, the HLA-DR molecule may be of allele DRB1*0101, DRB1*0301, DRB1*0401, DRB1*0701, DRB1*0801, DRB1*1101, DRB1*1301, DRB1*1501, DRB3*0101, DRB3*0202, DRB4*0101 or DRB5*0101. In instances in which the first MHC molecule of the target MHC molecule complex and/or the second MHC molecule of the non-target MHC molecule complex is a HLA-DP molecule, the HLA-DP molecule may be of allele DPA1*0103, DPA1*0202, DPAB1*0401 or DPAB1*0402. In instances in which the first MHC molecule of the target MHC molecule complex and/or the second MHC molecule of the non-target MHC molecule complex is a HLA-DQ molecule, the HLA-DQ molecule may be of allele DQA1*0101, DQB1*0301 or DQB1*0402.

In some instances, the first MHC molecule of the target MHC molecule complex and/or the second MHC molecule of the non-target MHC molecule complex may be an MHC class I-related protein (MR1) molecule or an antigen-presenting molecule CD1. Examples of human CD1 molecules include CD1a, CD1b, and CD1c molecules. The MR1, CD1, e.g., CD1a, CD1b and CD1c molecules may be useful as MHC molecules in target and/or non-target MHC molecule complexes wherein the MHC molecule is bound a lipid or small molecule.

The first MHC molecule of the target MHC molecule complex and/or the second MHC molecule of the non-target MHC molecule complex may be mouse MHC molecules. In instances in which the first MHC molecule of the target MHC molecule complex and/or the second MHC molecule of the non-target MHC molecule complex is a mouse MHC molecule, the mouse MHC molecule may be a mouse MHC class I molecule, such as a H-2K, H-2D, or H-2L molecule. In some instances, the mouse MHC molecule may be mouse MHC class 1b molecule, such as a Qa-2 or Qa-1 molecule. In other instances, the mouse MHC molecule may be mouse MHC class II molecule, such as a I-A or I-E molecule.

In any of methods, the first MHC molecule of the target MHC molecule complex and the second MHC molecule of the non-target MHC molecule complex may be of the same allele or of different alleles. The first MHC molecule of the target MHC molecule complex and the second MHC molecule of the non-target MHC molecule complex may both be MHC class I molecules. In instances in which both are MHC class I molecules, they may be MHC class I molecules of the same or different alleles. The first MHC molecule of the target MHC molecule complex and the second MHC molecule of the non-target MHC molecule complex may both be MHC class II molecules. In instances in which both are MHC class II molecules, they may be of the same or different alleles. The first MHC molecule of the target MHC molecule complex may be an MHC class I molecule and the second MHC molecule of the non-target MHC molecule complex may be an MHC class II molecule. The first MHC molecule of the target MHC molecule complex may be an MHC class II molecule and the second MHC molecule of the non-target MHC molecule complex may be an MHC class I molecule.

The first MHC molecule of the target MHC molecule complex, included in the plurality of MHC molecule complexes of the reaction mixture, may be bound to a target antigenic peptide. The target antigenic peptide may be a peptide, or peptide fragment, of any target antigen to which binding by an ABM is desirable. As discussed earlier herein, the target antigenic peptide may be a peptide, or peptide fragment of a target antigen that may be associated with an infectious agent, such as a viral, bacterial, parasitic, protozoal, or prion agent. It may be a peptide or peptide fragment of a target antigen associated with a tumor or a cancer, e.g., growth factor receptor or transcription factor. Further, it may be a peptide or peptide fragment of a target antigen associated with a degenerative condition or disease. As discussed herein, in some embodiments, the first MHC molecule of the target MHC molecule complex may be bound to a lipid or a small molecule.

The second MHC molecule of the non-target MHC molecule complex, included in the plurality of MHC molecules complexes, may be bound to a control peptide. In instances in which the non-target MHC complex is bound to a control peptide, the control peptide may be a scrambled peptide, serum albumin peptide, a heteroclitic peptide, or peptide to which immune cells of the sample are naïve. The scrambled peptide may have the same amino acid residue composition as a target antigenic peptide (bound to the first MHC molecule of the target MHC molecule complex), wherein the amino acid residues are presented in a different, e.g., scrambled, order relative to that of the target antigenic peptide. The serum albumin peptide may be a human or mouse serum albumin peptide. The control peptide may be any peptide, e.g., not only a serum albumin peptide, to which the ABMs of the plurality of immune cells would not be expected to bind, e.g., cardiolipin, keyhole limpet hemocyanin, flagellin or insulin. In instances in which the control peptide is a peptide to which ABMs of the plurality of immune cells would not be expected to bind, the control peptide may be a peptide of an abundantly expressed self-antigen of a subject from which the plurality of immune cells had been obtained. In other instances in which the control peptide is a peptide to which ABMs of the plurality of immune cells would not be expected to bind, the control peptide may be a peptide or peptide fragment of an antigen to which the plurality of immune cells are naïve. For example, the control peptide may be a peptide or peptide fragment of an antigen of a virus, e.g. HIV (e.g., TPGPGVRYPL (SEQ ID NO: 16)), if the subject from which the plurality of immune cells have been obtained, has not been exposed to the virus, e.g., HIV. For other example, the control peptide may be a heteroclitic peptide. Heteroclitic peptides may include peptides having valine, or leucine or other suitable residues at positions that anchor the peptide to the second MHC molecule, e.g., position 2 and/or a C-terminal residue, but alanine residues at the remaining amino acid positions (e.g., ALAAAAAAV (SEQ ID NO: 9), ATAAAAAAK (SEQ ID NO: 10), AYAAAAAAL (SEQ ID NO: 11), APAAAAAAV (SEQ ID NO: 12) or RYAAAAALL (SEQ ID NO: 13)). Additional examples of negative control peptides include ASYAAAAV (SEQ ID NO: 14) and vaccinia virus peptide TSYKFESV (SEQ ID NO: 15).

The target antigenic peptide bound to the first MHC molecule (of the target MHC molecule complexes) and/or the control peptide that may be bound to the second MHC molecule (of the non-target MHC molecule complexes), may be of any suitable length. The target antigenic peptide and/or control peptide may be of a length selected for optimal binding to a particular MHC molecule's, e.g., specific allele's, peptide binding groove. The target antigenic and/or control peptide may be at least 5, 6, 7, 8, 9, 10, 11, 12, 13, 14, 15, 16, 17, 18, 19, 20, 21, 22, 23, 24, 25, 26, 27, 28, 29, or 30 amino acids in length. The target antigenic and/or control peptide may be at most about 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, or 5 amino acids in length. The target antigenic and/or control peptide may be between about 5 and 35, between about 6 and 34, between about 7 and 33, between about 8 and 32, between about 9 and 31, between about 10 and 30, between about 11 and 29, between about 12 and 28, between about 13 and 27, between about 14 and 26, between about 15 and 25, between about 16 and 24, between about 17 and 23, or between about 18 and 22 amino acids in length.

A target antigenic and/or control peptide bound to an MHC class I molecule may be between about 6 to 12 amino acids in length, e.g., between about 7 to 11 amino acids in length, or between about 8 to 10 amino acids in length. A target antigenic and/or control peptide bound to an MHC class II molecule may be between about 5 to 35 amino acids in length, between about 10 to 30 amino acids in length, between about 15 to 25 amino acids in length, or between about 13 and 25 amino acids in length.

The target antigenic peptide bound to the first MHC molecule (of the target MHC molecule complexes) and/or the control peptide that may be bound to the second MHC molecule (of the non-target MHC molecule complexes) may be a peptide having a sequence selected/derived from a target or a control antigen by any, e.g., computational prediction, method. A computational prediction method for selection of the antigenic target peptide or control peptide, from the sequence of the target or control antigen, may be one based on an artificial learning system that uses, e.g., motif-based methods, machine learning methods, semi-supervised machine learning methods, or combinations thereof. A motif-based method for target antigenic and/or control peptide selection may be one based on a position weight matrix to model a gapless multiple sequence alignment of peptides. A Machine learning method may be one based on artificial neural networks. Examples of neural networks that may be used to select a peptide, e.g., target antigenic peptide or control peptide, from a target or control antigen include Pepdist, MHCflurry, NetMHC, NetMHCpan, NetMHCpan4.0, MixMHCpred 2.0.1, NetMHCcons 1.1, NetMHCII, NetMHCIIpan and PUFFIN.

### Exemplary Spatial Methodologies for Immune Profiling

In some embodiments of any of the spatial profiling methods described herein, the methods are used to identify and/or characterize antigen-binding molecules, which may be expressed by immune cells in the biological sample. Immune cells express various adaptive immunological receptors relating to immune function, such as T cell receptors (TCRs) and B cell receptors (BCRs). T cell receptors and B cell receptors play a part in the immune response by specifically recognizing and binding to antigens and aiding in their destruction. Various approaches for identifying and/or characterizing antigen-binding molecules using spatial analysis methods on a spatial array are described below.

A fundamental understanding of spatial heterogeneity with respect to T-cell receptor (TCR) and B-cell receptor (BCR) clonotypes within a biological sample is needed to understand multiple facets of their functionality, including, for example, which cells a particular TCR or BCR may be interacting with within the biological sample, the identity of TCR and/or BCR clonotypes in a given biological sample, and/or the identity of TCR and/or BCR clonotypes that are autoreactive in different autoimmune disorders. Numerous single-cell sequencing approaches can identify TCR and BCR clonotypes from a biological sample, however, at present methods are needed to link TCR and BCR sequences to spatial locations within a biological sample. Additionally, identifying the clonal regions, that is, regions defined by the places where variable (V), diverse (D), and joining (J) segments join to form the complementarity determining regions, including CDR1, CDR2, and CDR3, which provide specificity to the TCRs and/or BCRs, would greatly benefit the scientific arts. By coupling clonal information to spatial information, it is possible to understand which T-cell and B-cell clonotypes may be specifically interacting with given antigens or cell types within a biological sample.

However, capturing analytes encoding immune cell receptors can provide unique challenges. For example, spatially capturing the TCR and BCR gene components with sufficient efficiency to profile the majority of clonotypes in a given tissue is difficult. Capturing analytes encoding immune cell receptors with conventional short-read sequencing methods can result in a loss of sequenced regions that are more than about 1 kb away from the point where sequencing starts (e.g., 5′ end proximal regions comprising CDR sequences, such as CDR3). Linking separate TCR or BCR gene components that together form a complete receptor using sequencing data from spots containing multiple different cells are challenges addressed by the methods described herein.

Exemplary methods for the spatial analysis of ABMs (e.g., TCRs and/or BCRs) which can be used in accordance with the methods disclosed herein are described in, for example, PCT Application Publication Nos. WO 2021/247568 A1, WO 2023/086880 A1, and WO 2021/247543 A2, each of which are entirely incorporated herein by reference for all purposes.

### ABM Analysis Using a Spatial Transcriptomics for Antigen Receptors Approach

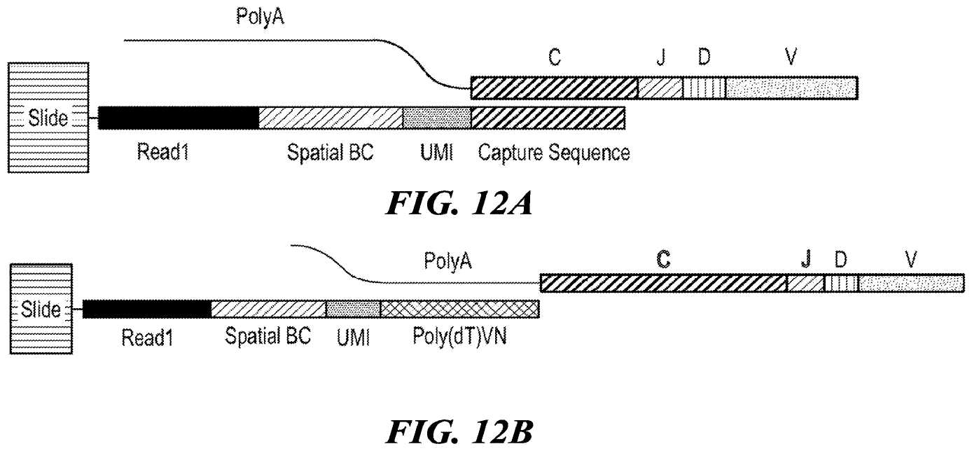

In particular embodiments, the capture sequence or capture domain of a capture probe on a spatial array binds (e.g., hybridizes) specifically to a nucleic acid sequence encoding a region of an ABM. An exemplary capture probe with a capture sequence that specifically binds to a nucleic acid sequence encoding a constant region of an ABM is depicted in FIG. 12A. In some embodiments, the ABM is selected from: a TCR alpha chain, a TCR beta chain, a TCR gamma chain, a TCR delta chain an immunoglobulin kappa light chain, an immunoglobulin lambda light chain, an immunoglobulin heavy chain. In some embodiments, the capture sequence/capture domain binds specifically to a nucleic acid sequence encoding a constant region of the T cell receptor alpha chain. In some embodiments, the capture sequence binds specifically to a nucleic acid sequence encoding a constant region of the T cell receptor beta chain. In some embodiments, the capture sequence binds specifically to a nucleic acid sequence encoding a constant region of the T cell receptor delta chain. In some embodiments, the capture sequence binds specifically to a nucleic acid sequence encoding a constant region of the T cell receptor gamma chain. In some embodiments, the capture sequence binds specifically to a nucleic acid sequence encoding a constant region of the immunoglobulin kappa light chain. In some embodiments, the capture sequence binds specifically to a nucleic acid sequence encoding a constant region of the immunoglobulin lambda light chain. In some embodiments, the capture sequence binds specifically to a nucleic acid sequence encoding a constant region of the immunoglobulin heavy chain.

In other embodiments, the capture sequence is a homopolymeric sequence, e.g., a polyT sequence. FIG. 12B shows an exemplary poly(A) capture with a poly(T) capture domain. A poly(T) capture domain can capture other analytes, such as during global mRNA capture, including analytes encoding ABMs within the tissue sample. For example, in some embodiments, a poly(T) capture domain a hybridizes to a nucleic acid sequence encoding all or a portion of a TCR alpha chain, a TCR beta chain, a TCR gamma chain, a TCR delta chain an immunoglobulin kappa light chain, an immunoglobulin lambda light chain, or an immunoglobulin heavy chain. In some embodiments, following capture of analytes by capture probes, capture probes can be extended, e.g., via reverse transcription. Second strand synthesis can generate double stranded cDNA products that are spatially barcoded. The double stranded cDNA products, which may comprise ABM encoding sequences and non-ABM related analytes, can be enriched for ABM encoding sequences.

An exemplary enrichment workflow may comprise amplifying the cDNA products (or amplicons thereof) with a first primer that specifically binds to a functional sequence of the first capture probe or reverse complement thereof and a second primer that binds to a nucleic acid sequence encoding a variable region of the ABM expressed by the ABM-expressing cell or reverse complement thereof. In some embodiments, the first primer and the second primer flank the spatial barcode of the first spatially barcoded polynucleotide or amplicon thereof. In some embodiments, the first primer and the second primer flank a J junction, a D junction, and/or a V junction.

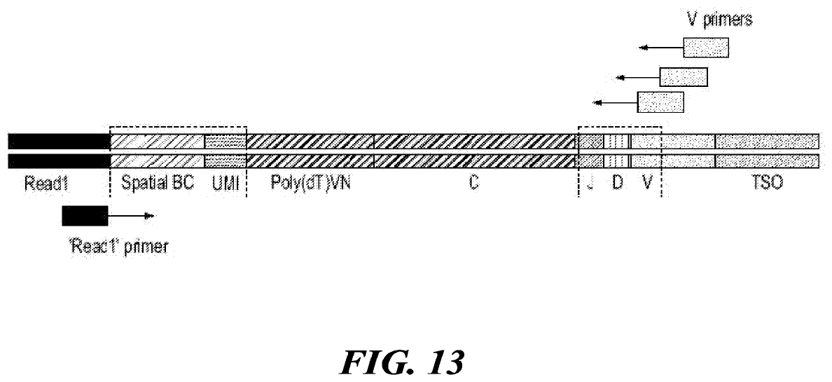

FIG. 13 shows an exemplary analyte enrichment strategy following analyte capture on the array. The portion of the immune cell analyte of interest includes the sequence of the V(D)J region, including CDR sequences. As described herein, a poly(T) capture probe captures an analyte encoding an ABM, an extended capture probe is generated by a reverse transcription reaction, and a second strand is generated. The resulting nucleic acid library can be enriched by the exemplary scheme shown in FIG. 13, where an amplification reaction including a Read 1 primer complementary to the Read 1 sequence of the capture probe and a primer complementary to a portion of the variable region of the immune cell analyte, can enrich the library via PCR. While FIG. 13 depicts a Read 1 primer, it is understood that a primer complementary to other functional sequences, such as other sequencing primer sequences, or sequencer specific flow cell attachment sequences, or portions of such functional sequences, may also be used. While FIG. 13 depicts a polyT capture sequence, it is understood that other capture sequences disclosed herein may be present in library members. The enriched library can be further enriched by nested primers complementary to a portion of the variable region internal (e.g., 5′) to the initial variable region primer for practicing nested PCR.

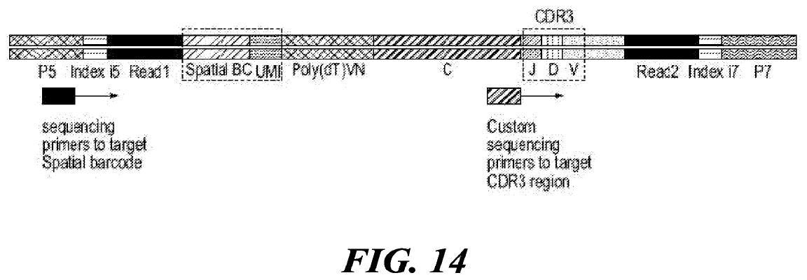

FIG. 14 shows a sequencing strategy with a primer specific complementary to the sequencing flow cell attachment sequence (e.g., P5) and a custom sequencing primer complementary to a portion of the constant region of the analyte. This sequencing strategy targets the constant region to obtain the sequence of the CDR regions, including CDR3, while concurrently or sequentially sequencing the spatial barcode (BC) and/or unique molecular identifier (UMI) of the capture probe. By capturing the sequence of a spatial barcode, UMI and a V(D)J region the receptor is not only determined, but its spatial location and abundance within a cell or tissue is also identified.

In some embodiments, the method includes (a) providing an array comprising a plurality of capture probes, wherein a capture probe of the plurality of capture probes comprises (i) a spatial barcode and (ii) a capture domain that hybridizes to a poly(A) sequence of a nucleic acid encoding an immune cell receptor expressed by an immune cell in the biological sample; (b) hybridizing the capture domain to the nucleic acid encoding the immune cell receptor; (c) extending the capture probe using the nucleic acid encoding the immune cell receptor as a template to generate an extended capture probe comprising a sequence encoding a CDR (e.g., CDR1, 2 and/or 3), or a complement thereof, of the immune cell receptor; (d) hybridizing one or more enrichment probes to the extended capture probe, or a complement thereof, in a portion encoding a constant region of the immune cell receptor, wherein the one or more enrichment probes comprises a binding moiety capable of binding a capture moiety; (e) enriching the extended capture probe or the complement thereof via an interaction between the binding moiety in the one or more enrichment probes and the capture moiety; and (f) determining (i) the sequence of the spatial barcode or a complement thereof, and (ii) all or a portion of the sequence of the nucleic acid encoding the immune cell receptor or a complement thereof, and using the determined sequences of (i) and (ii) to determine the location of the immune cell receptor in the biological sample.

In some embodiments, the one or more enrichment probes hybridizes to a nucleic acid sequence encoding a constant region of the immune cell receptor (e.g., BCR or TCR), or a complement thereof. In some embodiments, step (f) comprises determining a sequence encoding one or more of CDR1, CDR2, and CDR3 of the immune cell receptor, and optionally, determining a sequence encoding a full-length variable domain of the immune cell receptor. In some embodiments, the method further includes generating the complement of the extended capture probe using the extended capture probe as a template, wherein the complement of the extended capture probe comprises (i) a sequence that is complementary to the spatial barcode, and (ii) a sequence that corresponds to all or a portion of the sequence of the nucleic acid encoding the immune cell receptor. In some embodiments, the binding moiety comprises biotin and the capture moiety comprises streptavidin.

In some embodiments, the determining in step (f) comprises sequencing the extended capture probe or the complement thereof to determine (i) the sequence of the spatial barcode, or the complement thereof, and (ii) all or a portion of the sequence of the nucleic acid encoding the immune cell receptor of the immune cell clonotype or the complement thereof. In some embodiments, the sequencing comprises long read sequencing.

In some embodiments, the capture probe further comprises an adaptor domain and the method further comprises after step (e), performing a polymerase chain reaction using i) a first primer complementary to the adaptor domain of the capture probe, and ii) a second primer complementary to a portion of a nucleic acid sequence encoding a variable region of the immune cell receptor. In some embodiments, the second primer is complementary to a nucleic acid sequence 5′ to the sequence encoding a CDR (e.g., CDR1, 2 or 3) of the immune cell receptor. In some embodiments, generating the complement of the extended capture probe comprises use of a template switch oligonucleotide. By using capture domains that hybridize to a poly(A) sequences, the method is advantageous in that it allows for spatial analysis of global mRNA expression as well as a targeted spatial analysis of ABMs at once. In some embodiments, the method includes (a) providing an array comprising a plurality of capture probes, wherein a capture probe of the plurality of capture probes comprises: (i) a spatial barcode and (ii) a capture domain that hybridizes to a nucleic acid encoding the immune cell receptor; (b) hybridizing the capture domain of the capture probe to the nucleic acid encoding the immune cell receptor; (c) extending the capture probe using the nucleic acid encoding the immune cell receptor as a template, thereby generating an extended capture probe; (d) hybridizing one or more enrichment probes to the extended capture probe, or a complement thereof, in a portion encoding a constant region of the immune cell receptor; (e) enriching the extended capture probe, or the complement thereof, via the one or more enrichment probes; and

(f) determining (i) the sequence of the spatial barcode or a complement thereof, and (ii) all or a portion of the sequence of the nucleic acid encoding the immune cell receptor or a complement thereof, and using the determined sequences of (i) and (ii) to determine the location of the immune cell receptor in the biological sample. In some embodiments, the method further includes contacting the biological sample with the array or bringing the biological sample into proximity of the array.

### ABM Analysis Using Nucleic Acid Library Methods to Remove Portion of Analyte Sequences

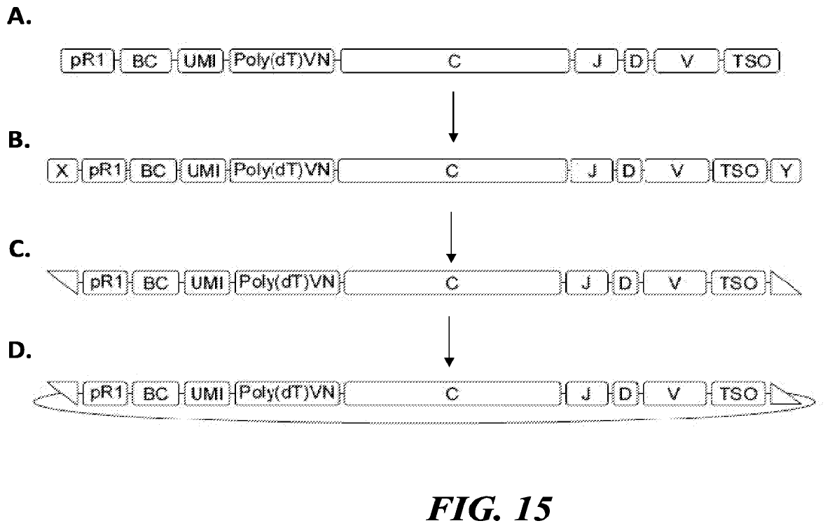

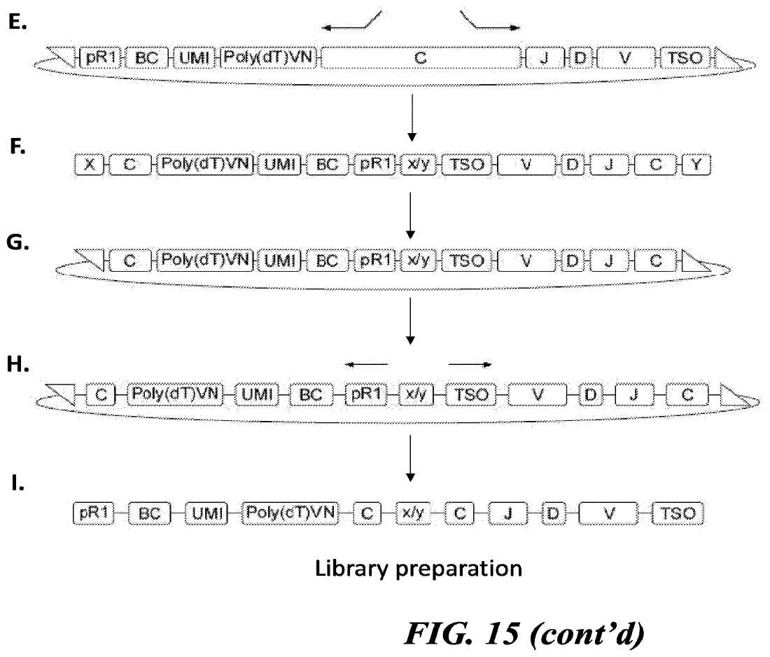

FIG. 15 shows an exemplary nucleic acid library preparation method to remove a portion of an analyte sequence via double circularization of a member of a nucleic acid library. Panel A shows an exemplary member of a nucleic acid library including, in a 5′ to 3′ direction, a first adaptor (e.g., primer sequence R1, pR1 (e.g., Read 1)), a barcode (e.g., a spatial barcode or a cell barcode), a unique molecular identifier (UMI), a capture domain (e.g., poly(T) VN sequence), a sequence complementary to a nucleic acid analyte encoding an ABM (e.g., encoding C, J, D and V regions of a BCR or TCR), and a second adaptor (e.g., template switching oligonucleotide sequence (TSO)). For purposes of this example an analyte including a constant region (C) and V(D)J sequence are shown, however, the methods described herein can be equally applied to other analyte sequences in a nucleic acid library. Panel B shows the exemplary member of a nucleic acid library where additional sequences can be added to both the 3′ and 5′ ends of the nucleic acid member (shown as a X and Y) via a PCR reaction. The additional sequences added can include a recognition sequence for a restriction enzyme (e.g., restriction endonuclease). The restriction recognition sequence can be for a rare restriction enzyme. The exemplary member of the nucleic acid library shown in Panel B can be digested with a restriction enzyme to generate sticky ends shown in Panel C (shown as triangles) and can be intramolecularly circularized by ligation to generate the circularized member of the nucleic acid library shown in Panel D. The ligation can be performed with a DNA ligase. The ligase can be T4 ligase. A primer pair can be hybridized to a circularized nucleic acid member, where a first primer hybridizes to a 3′ portion of a sequence encoding the constant region (C) and includes a second restriction enzyme (e.g., restriction endonuclease) sequence that is non-complementary to the analyte sequence, and where a second primer hybridized to a 5′ portion of a sequence encoding the constant region (C), and where the second primer includes a second restriction enzyme sequence (Panel E). The first primer and the second primer can generate a linear amplification product (e.g., a first double-stranded nucleic acid product) as shown in Panel F, which includes the second restriction enzyme recognition sequences (shown as X and Y end sequences). The linear amplification product (Panel F) can be digested with a second restriction enzyme to generate sticky ends and can be intramolecularly ligated with a ligase (e.g., T4 DNA ligase) to generate a second double-stranded circularized nucleic acid product as shown in Panel G. The second double-stranded circularized nucleic product (Panel G) can be amplified with a third primer, pR1, substantially complementary to the first adaptor (e.g., Read 1) sequence and a fourth primer substantially complementary to the second adapter (e.g., TSO) as shown in Panel H to generate a version of the double-stranded member of the nucleic acid library lacking all, or a portion of, the sequence encoding the constant region (C) of the ABM encoded by the nucleic acid analyte (Panel I). The resulting double-stranded member of the nucleic acid library lacking all or a portion of the analyte sequence encoding the constant region can undergo library preparation methods, such as library preparation methods used in single-cell or spatial analyses. For example, the double-stranded member of the nucleic acid library lacking all, or a portion of, the analyte sequence encoding the constant region of the ABM can be fragmented, followed by end repair, a-tailing, adaptor ligation, and/or additional amplification (e.g., PCR). The fragments can then be sequenced using, for example, paired-end sequencing using TruSeq Read 1 and TruSeq Read 2 as sequencing primer sites or any other sequencing method described herein. As such, sequences can be determined from regions more than about 1 kb away from the end of a nucleic acid analyte (e.g., 3′ end) encoding an ABM and can link such a sequence to a barcode sequence (e.g., a spatial barcode, a cell barcode) in library preparation methods (e.g., sequencing preparation). For purposes of this example a nucleic acid analyte encoding a constant region (C) and V(D)J region of an ABM (e.g., BCR or TCR) are shown, however, the methods described herein can be equally applied to other analyte sequences in a nucleic acid library.

An exemplary member of a nucleic acid library can be prepared as shown in FIG. 15 to generate a first double-stranded circularized nucleic acid product shown in Panel D of FIG. 15 as previously described.

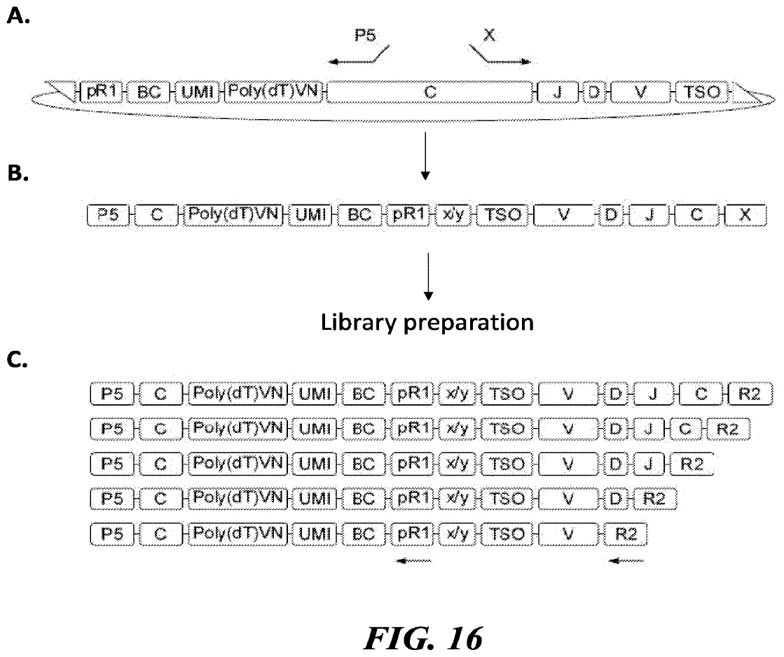

FIG. 16 depicts another exemplary workflow for processing such double-stranded circularized nucleic acid product. A primer pair can be contacted with the double-stranded circularized nucleic acid produce with a first primer that can hybridize to a sequence from a 3′ region of the sequence encoding the constant region of the ABM and a sequence including a first functional domain (e.g., P5). The second primer can hybridize to a sequence from a 5′ region of the analyte sequence encoding the constant region of the analyte, and includes a sequence including a second functional domain (shown as “X”) as shown in Panel A. Amplification of the double-stranded circularized nucleic acid product results in a linear product as shown in Panel B, where all, or a portion of, the constant region (C) is removed. The first functional domain can include a sequencer specific flow cell attachment sequence (e.g., P5). The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the analyte sequence encoding the constant region of the ABM can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites (Panel C, arrows), or any other sequencing method described herein. After library preparation methods described herein, a different sequencing primer for the first adaptor (e.g., Read 1) is used since the orientation of the first adaptor (e.g., Read 1) sequence will be reversed. Accordingly, sequences can be determined from regions more than about 1 kb away from the end of a nucleic acid analyte (e.g., 3′ end) encoding an ABM and can link such a sequence to a barcode sequence (e.g., a spatial barcode, a cell barcode) in further library preparation methods (e.g., sequencing preparation). For purposes of this example, a nucleic acid analyte encoding a constant region (C) and V(D)J region of an ABM are shown, however, the methods described herein can be applied to other analyte sequences in a nucleic acid library as well.

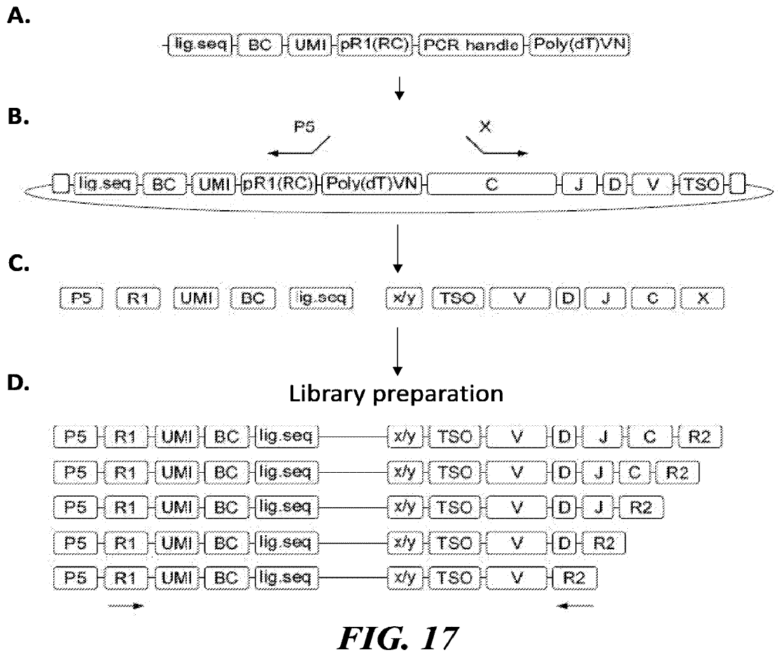

FIG. 17 shows an exemplary nucleic acid library preparation method to remove all or a portion of a constant sequence of a nucleic acid analyte from a member of a nucleic acid library via circularization. Panels A and B shows an exemplary member of a nucleic acid library including, in a 5′ to 3′ direction, a ligation sequence, a barcode sequence, a unique molecular identifier, a reverse complement of a first adaptor (e.g., primer sequence pR1 (e.g., Read 1)), a capture domain, a sequence complementary to the nucleic acid analyte encoding an ABM, and a second adapter (e.g., TSO sequence). The ends of the double-stranded nucleic acid can be ligated together via a ligation reaction where the ligation sequence splints the ligation to generate a circularized double-stranded nucleic acid as shown in Panel B. The circularized double-stranded nucleic acid can be amplified with a pair of primers to generate a linear nucleic acid product lacking all or a portion of the constant region of the analyte sequence encoding the constant region of the ABM (Panels B and C). The first primer can include a sequence substantially complementary to the reverse complement of the first adaptor and a first functional domain. The first functional domain can be a sequencer specific flow cell attachment sequence (e.g., P5). The second primer can include a sequence substantially complementary to a sequence from a 5′ region of the analyte sequence encoding the constant region of the ABM, and a second functional domain. The second functional domain can include an amplification domain such as a primer sequence to amplify the nucleic acid library prior to further sequencing preparation. The resulting double-stranded member of the nucleic acid library lacking all or a portion of the constant region can undergo library preparation methods, such as library preparation methods used in single-cell or spatial analyses. For example, the double-stranded member of the nucleic acid library lacking all, or a portion of, the sequence encoding the constant region of the ABM can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein (Panel D). After library preparation methods (e.g., described herein), sequencing primers can be used since the orientation of Read 1 will be in the proper orientation for sequencing primer pR1. Accordingly, sequences can be determined from regions more than about 1 kb away from the end of a nucleic acid analyte (e.g., 3′ end) encoding an ABM and can link such a sequence to a barcode sequence (e.g., a spatial barcode, a cell barcode) in further library preparation methods (e.g., sequencing preparation). For purposes of this example, a nucleic acid analyte encoding a constant region (C) and V(D)J region are shown, however, the methods described herein can be applied to other analyte sequences in a nucleic acid library as well.

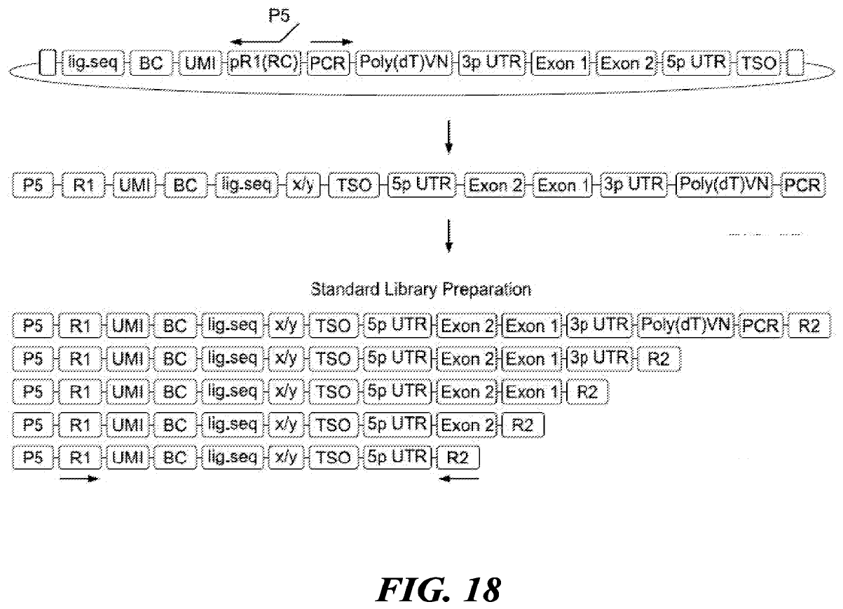

FIG. 18 shows an exemplary nucleic acid library method to reverse the orientation of an analyte sequence in a member of a nucleic acid library. Panel A shows an exemplary member of a nucleic acid library including, in a 5′ to 3′ direction, a ligation sequence, a barcode (e.g., a spatial barcode or a cell barcode), unique molecular identifier, a reverse complement of a first adaptor, an amplification domain, a capture domain, a sequence of a nucleic acid analyte encoding an ABM, and a second adapter. The ends of the double-stranded nucleic acid can be ligated together via a ligation reaction where the ligation sequence splints the ligation to generate a circularized double-stranded nucleic acid also shown in Panel A. The circularized double-stranded nucleic acid can be amplified to generate a linearized double-stranded nucleic acid product, where the orientation of the nucleic acid analyte is reversed such that the 5′ sequence (e.g., 5′ UTR) is brought in closer proximity to the barcode (e.g., a spatial barcode or a cell barcode) (Panel B). The first primer includes a sequence substantially complementary to the reverse complement of the first adaptor and a functional domain. The functional domain can be a sequencer specific flow cell attachment sequence (e.g., P5). The second primer includes a sequence substantially complementary to the amplification domain. The resulting double-stranded member of the nucleic acid library including a reversed analyte sequence (e.g., the 5′ end of the analyte sequence is brought in closer proximity to the barcode) can undergo library preparation methods, such as library preparation methods used in single-cell or spatial analyses. For example, the double-stranded member of the nucleic acid library lacking all, or a portion of, the nucleic acid analyte sequence encoding the constant region of the ABM can be fragmented, followed by end repair, A-tailing, adaptor ligation, and/or amplification (e.g., PCR) (Panel C). The fragments can then be sequenced using, for example, paired-end sequencing using TruSeq Read 1 and TruSeq Read 2 as sequencing primer sites, or any other sequencing method described herein. Accordingly, sequences from the 5′ end of a nucleic acid analyte encoding an ABM will be included in sequencing libraries (e.g., paired end sequencing libraries). Any type of analyte sequence in a nucleic acid library can be prepared by the methods described in this Example (e.g., reversed).

### Spatial Analysis Using Array Features Comprising Poly(T) and Poly(GI) Capture Domains

Provided herein are methods of determining a location of a target nucleic acid encoding an ABM in a biological sample that include: (a) contacting the biological sample with an array comprising a feature, where the feature comprises an attached first and second probe, wherein: a 5′ end of the first probe is attached to the feature; the first probe comprises in a 5′ to a 3′ direction: a spatial barcode and a poly(T) capture domain, where the poly(T) capture domain binds specifically to the target nucleic acid; a 5′ end of the second probe is attached to the feature; a 3′ end of the second probe is reversibly blocked; and the second probe comprises a poly(GI) capture domain; (b) extending a 3′ end of the first probe to add a sequence that is complementary to a portion of the target nucleic acid; (c) ligating an adapter to the 5′ end of the target nucleic acid specifically bound to the first probe; (d) adding a sequence complementary to the adapter to the 3′ end of the first probe; (e) adding non-templated cytosines to the 3′ end of the first probe to generate a poly(C) sequence, where the poly(C) sequence specifically binds to the poly(GI) capture domain of the second probe; (f) unblocking the 3′ end of the second probe and extending the 3′ end of the second probe to add a sequence comprising a sequence in the target nucleic acid and a sequence that is complementary to the spatial barcode; (g) cleaving a region of the second probe at a cleavage site that is 5′ to the poly(GT) capture domain, thereby releasing the second probe from the feature; and (h) determining (i) all or a part of the sequence of the spatial barcode, or a complement thereof, and (ii) all or a part of the sequence of the target nucleic acid, or a complement thereof, and using the sequences of (i) and (ii) to determine the location of the target nucleic acid in the biological sample.

In some embodiments, a feature can include two or more pairs of a first and a second probe (e.g., any of the first and second probes described in this section). A first pair of a first and a second probe at a feature, as compared to a second pair of a first and a second probe at the feature, can have a different first and/or second probe as compared to first and/or second probe of the second pair (e.g., a different capture domain in the first probe and/or a different barcode in the first and/or second probes). In some embodiments, the spatial barcode in the first probe of the first pair and the spatial barcode in the first probe of the second pair are the same. In some embodiments, the spatial barcode in the first probe of the first pair and the spatial barcode in the first probe of the second pair are different. In some embodiments, the capture domain of the first probe of the first pair is the same as the capture domain of the first probe of the second pair. In some embodiments, the capture domain of the first probe of the first pair is different from the capture domain of the first probe of the second pair.

In some embodiments, the capture domain on the first probe has a poly(T) capture domain, where the poly(T) capture domain is configured to interact with the target nucleic acid (e.g., positioned at the 3′ end of the first probe). For example, the poly(T) capture domain specifically binds to a messenger RNA (mRNA), via the poly(A) tail of the mRNA. For example, a poly(T) capture domain can include at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, or at least 30 contiguous thymidines.

In some embodiments, the poly(GI) capture domain of the second probe is configured to interact with a poly(C) tail of an oligonucleotide, e.g., a poly(C) tail added to the 3′ end of the extended first probe. In some embodiments, the poly(C) tail is added to the 3′ end of the first probe after the extension of the first probe to add a sequence that is complementary to a portion of the target nucleic acid. In some embodiments, the poly(GI) capture domain comprises a sequence of at least 5 contiguous guanosine(s) and/or inosine(s). For example, a poly(GI) capture domain comprises a sequence of (GGI)n, wherein n is about 3 to about 20. In some embodiments, the poly(GI) capture domain comprises a sequence of (GGI)n, wherein n is 1, 2, 3, 4, 5, 6, 7, 8, 9, 10, 11, 12, 13, 14, 15, 16, 17, 18, 19, or 20. For example, a poly(GI) capture domain comprises a sequence of (GI)n, wherein n is about 4 to about 30. For example, a poly(GI) capture domain comprises a sequence of (GI)n, wherein n is 4, 5, 6, 7, 8, 9, 10, 11, 12, 13, 14, 15, 16, 17, 18, 19, 20, 21, 22, 23, 24, 25, 26, 27, 28, 29, or 30. For example, a poly(GI) capture domain comprises a sequence of (IG)n, wherein n is about 4 to about 30. For example, a poly(GI) capture domain comprises a sequence of (IG)n, wherein n is 4, 5, 6, 7, 8, 9, 10, 11, 12, 13, 14, 15, 16, 17, 18, 19, 20, 21, 22, 23, 24, 25, 26, 27, 28, 29, or 30.

In some embodiments, the second probe can comprise a spatial barcode, which is positioned 5′ to the poly(GI) capture domain. In some embodiments, the spatial barcode in the first probe is different from the spatial barcode sequence in the second probe. In some embodiments, the spatial barcode in the first probe is the same as the spatial barcode sequence in the second probe.

In some embodiments, both the first and the second probes are cleavable. In some embodiments, the first probe and the second probe have different cleavage sites and are cleavable using different methods. In some embodiments, the first probe and the second probe have the same cleavable site and are cleavable using the same method. In some embodiments, the cleavage domain of the first probe is 5′ to the poly(T) capture domain and/or the cleavage domain of the second probe is 5′ to the poly(GI) capture domain.

In some embodiments, the first probe is not cleavable and the second probe is cleavable. In some embodiments, the cleavage site of the second probe is 5′ to the poly(GI) capture domain of the second probe. In some embodiments, the cleavage site on the second probe is a uracil. In some embodiments, the uracil is cleaved by USER (Uracil-Specific Excision Reagent).

In some embodiments, the first probe further comprises a unique molecular identifier (UMI). In some embodiments, the second probe further comprises a unique molecular identifier (UMI). In some embodiments, the UMI in the first probe and the UMI in the second probe comprise different sequences. In some embodiments, the UMI in the first probe and the UMI in the second probe comprise the same sequence.

In some embodiments, step (h) includes sequencing all or a part of the sequence of the spatial barcode, or a complement thereof, and sequencing all of a part of the sequence of the target nucleic acid, or a complement thereof. The sequencing can be performed using any of the aforementioned methods. In some embodiments, step (h) includes sequencing the full-length sequence of the spatial barcode, or a complement thereof. In some embodiments, step (h) includes sequencing a part of the sequence of the spatial barcode, or a complement thereof. In some embodiments, step (h) includes sequencing the full-length sequence of the target nucleic acid, or a complements thereof. In some embodiments, step (h) includes sequencing a part of the target nucleic acid, or a complement thereof. In some embodiments, the sequencing is performed using high throughput sequencing. In some embodiments, the target nucleic acid is sequenced from the 5′ end of the target nucleic acid. In some embodiments, the target nucleic acid is sequenced from the 3′ end of the target nucleic acid. In some embodiments, the target nucleic acid is sequenced from both the 3′ end and the 5′ end of the target nucleic acid.

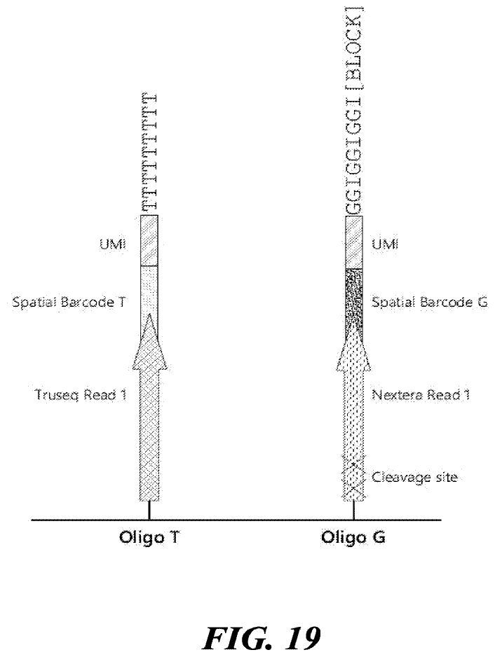

FIG. 19 is a schematic diagram showing an exemplary feature comprising an attached first and second probe. The first probe comprises in a 5′ to 3′ direction: a functional domain comprising a Truseq Read 1 primer, a spatial barcode, a UMI, and a poly(T) capture domain, where the poly(T) capture domain binds specifically to the target nucleic acid. The 5′ end of the first probe is attached to the feature.

The second probe comprises in a 5′ to 3′ direction: a cleavage domain, a functional domain comprising a Nextera Read 1 primer, a spatial barcode, a UMI, and a poly(GI) capture domain. The 5′ end of the second probe is attached to the feature. In some embodiments, the poly(GI) capture domain comprises a sequence of (GGI)n, wherein n is about 3 to about 20. In some embodiments, the poly(GI) capture domain comprises a sequence of (GGI)n, wherein n is 1, 2, 3, 4, 5, 6, 7, 8, 9, 10, 11, 12, 13, 14, 15, 16, 17, 18, 19, or 20. In some embodiments, the 3′ end of the second probe is reversibly blocked.

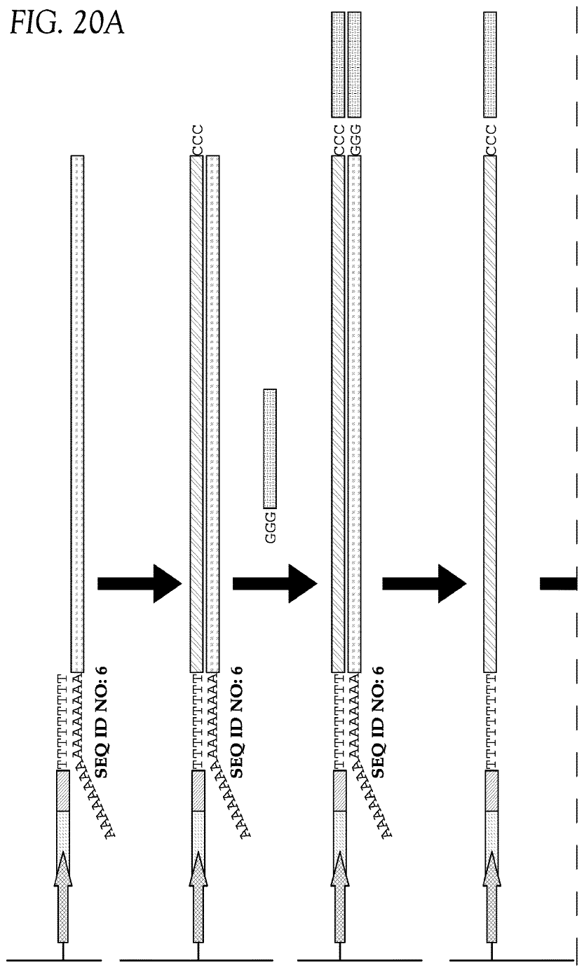

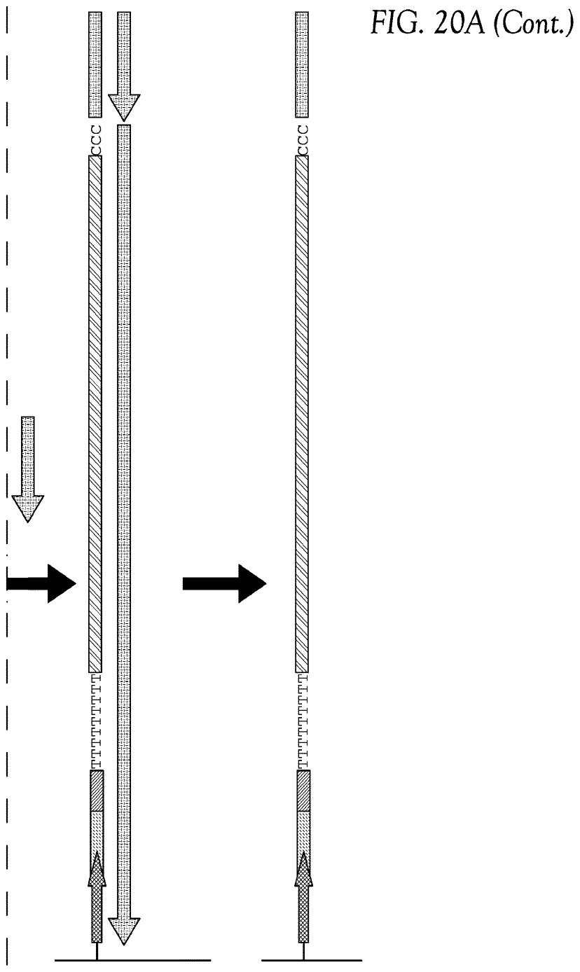

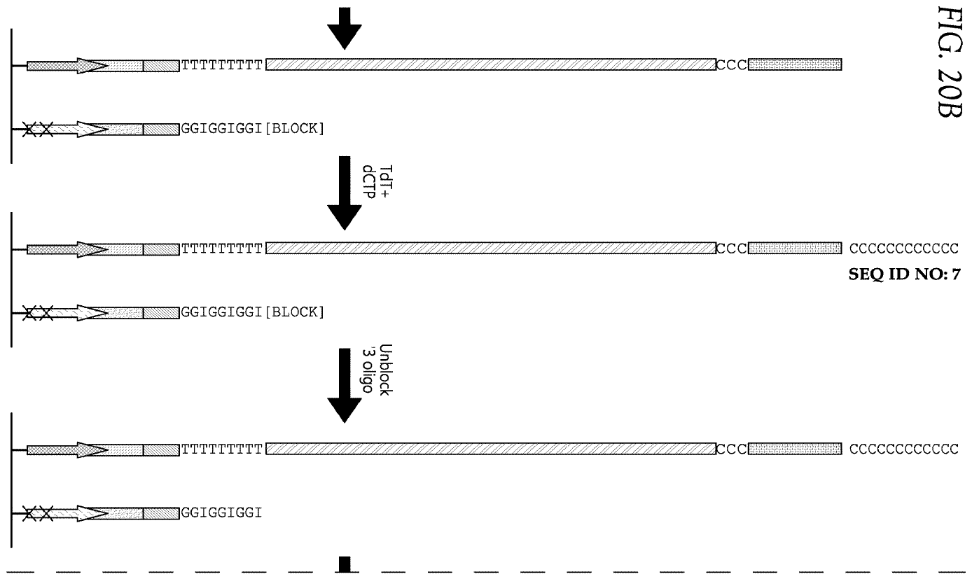

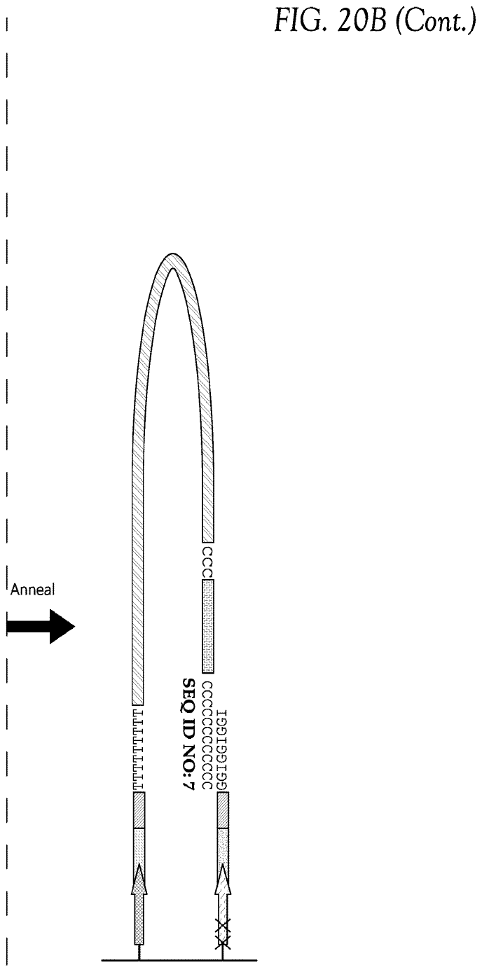

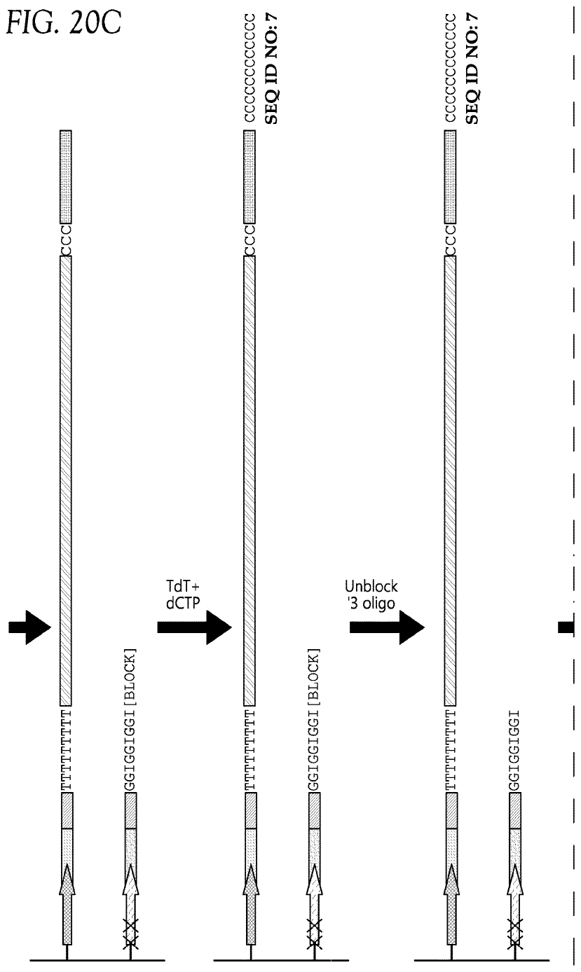

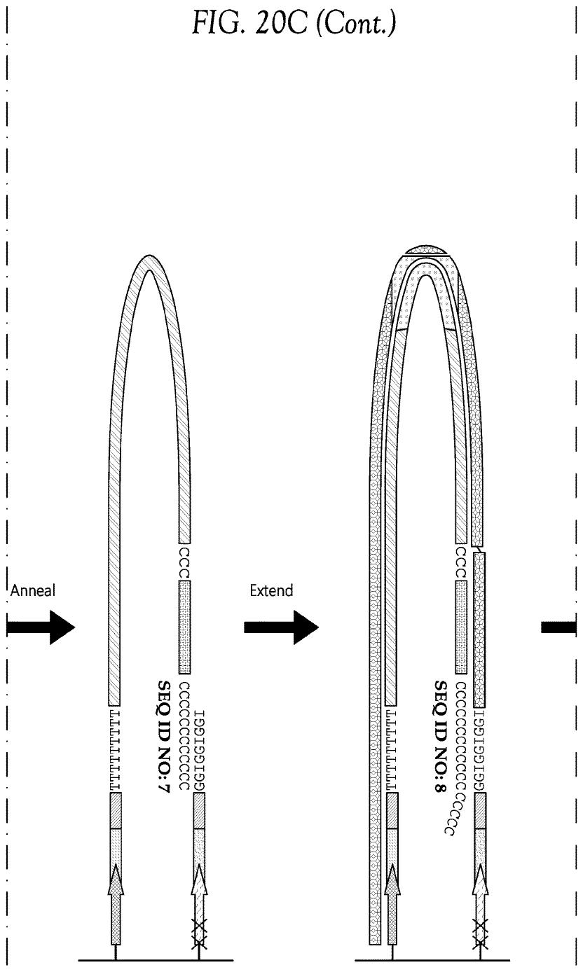

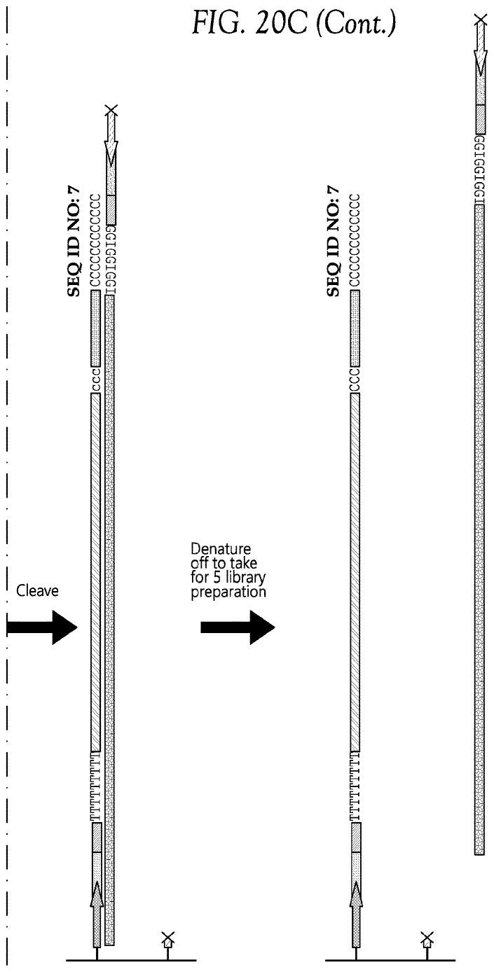

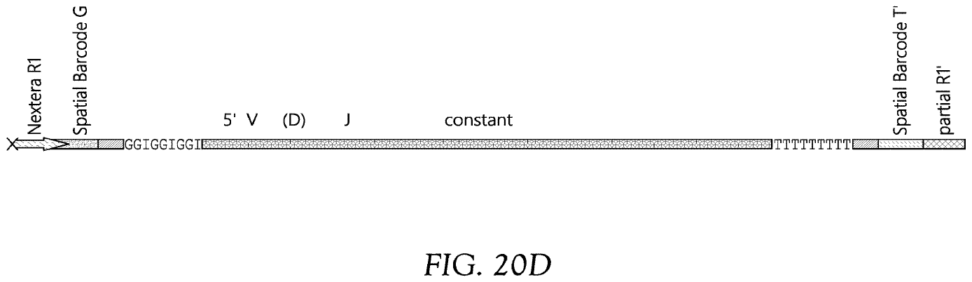

FIG. 20A is an exemplary diagram showing, from left to right, the annealing of the target analyte (e.g., target nucleic acid) to the poly(T) capture domain of the first probe; the extension of the first probe to add a sequence that is complementary to a portion of the target nucleic acid; the ligation of an adaptor to the 5′ end of the target nucleic acid specifically bound to the first probe; the addition of a sequence complementary to the adaptor to the 3′ end of the first probe; the releasing of the target nucleic acid from the first probe; the generation of a complement of the extended first probe; and the releasing of the complement of the extended first probe. In some embodiments, the released target nucleic acid is sequenced. In some embodiments, the released complement of the extended first probe is sequenced.

FIG. 20B is an exemplary diagram showing, from left to right, the addition of non-templated cytosines to the 3′ end of the extended first probe (e.g., extended to include a sequence that is complementary to part of a sequence of a target nucleic acid) to generate a poly(C) sequence, where the poly(C) sequence specifically binds to the poly(GI) capture domain of the second probe; the unblocking of the 3′ end of the second capture probe; and the hybridization of the poly(C) sequence on the 3′ end of the first probe to the poly(GI) capture domain at the 3′ end of the second probe.

In alternative embodiments, the second probe comprises a poly(T) capture domain and a poly(A) sequence is added to the 3′ end of the extended first probe (e.g., extended to add a sequence that is complementary to a portion of the sequence of a target nucleic acid), and the poly(A) sequence hybridizes to the poly(T) capture domain of the second probe.

The method allows for the sequencing of the target nucleic acid from either the 3′ end or the 5′ end, or both the 3′ and the 5′ ends of the target nucleic acid. For target nucleic acids that have large sizes (e.g., larger than 1 kb), the methods allow more accurate spatial sequence information to be obtained.

Also described in this section is an array comprising a feature, where the feature comprises an attached first and second probe, wherein: a 5′ end of the first probe is attached to the feature; the first probe comprises in a 5′ to a 3′ direction: a spatial barcode and a poly(T) capture domain, wherein the poly(T) capture domain binds specifically to the target nucleic acid; a 5′ end of the second probe is attached to the feature; a 3′ end of the second probe is reversibly blocked; and the second probe comprises a poly(GI) capture domain.

In some embodiments of any of the arrays described in this section, a feature can include two or more pairs of a first and a second probe (e.g., any of the first and second probes described in this section). A first pair of a first and a second probe at a feature, as compared to a second pair of a first and a second probe at the feature, can have a different first and/or second probe as compared to first and/or second probe of the second pair (e.g., a different capture domain in the first probe and/or a different barcode in the first and/or second probes). In some embodiments of any of the arrays described in this section, the spatial barcode in the first probe of the first pair and the spatial barcode in the first probe of the second pair are the same. In some embodiments of any of the arrays described in this section, the spatial barcode in the first probe of the first pair and the spatial barcode in the first probe of the second pair are different. In some embodiments of any of the arrays described in this section, the capture domain of the first probe of the first pair is the same as the capture domain of the first probe of the second pair. In some embodiments of any of the arrays described in this section, the capture domain of the first probe of the first pair is different from the capture domain of the first probe of the second pair.

### ABM Analysis Using 5′ Capture of Target Nucleic Acids

In some embodiments of a spatial immune profiling method, the analyte of the ABM-expressing cell is a cDNA of an mRNA transcript encoding the ABM. In some embodiments, the cDNA is generated by in situ reverse transcription of the mRNA encoding the ABM.

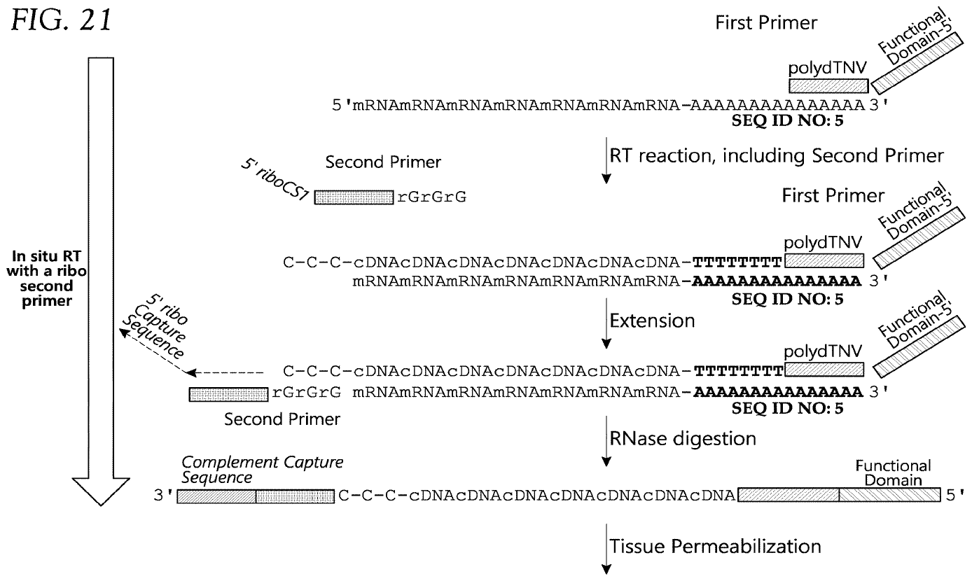

FIG. 21 is a schematic showing generation of a cDNA by in situ reverse transcription of a target nucleic acid (e.g., mRNA) from a first primer including a sequence complementary to the target nucleic acid and a functional domain and a second primer that includes a capture sequence and a sequence complementary a homopolynucleotide sequence. More specifically, target nucleic acids are contacted with a first primer that includes a sequence complementary to the target nucleic acid (e.g., poly(dT) sequence, a poly(dTNV) sequence) and a functional domain. In some examples, the functional domain is a primer binding site. In some examples, the functional domain is a sequencing specific site (e.g., Read2 site). The target nucleic acid is reverse transcribed and a homopolynucleotide sequence is added to the 3′ end of the cDNA.

A second primer is added where the second primer includes a sequence complementary to the homopolynucleotide sequence added to the 3′ end of the cDNA and a capture sequence. In some examples, the second primer includes an RNA sequence (e.g., a ribo-functional sequence such as a linker sequence, a primer binding sequence, a sequence for use in next generation sequencing, etc.). After reverse transcription and extension of the 3′ end of the cDNA using the second primer as an extension template, an RNase (e.g., RNase H) is contacted with the biological sample (e.g., a tissue section). The RNase degrades the RNA strand of the RNA/cDNA duplex, leaving a single-stranded cDNA product (e.g., an extension product) that includes the first primer at its 5′ end and a capture sequence capable of hybridizing a capture domain of a capture probe.

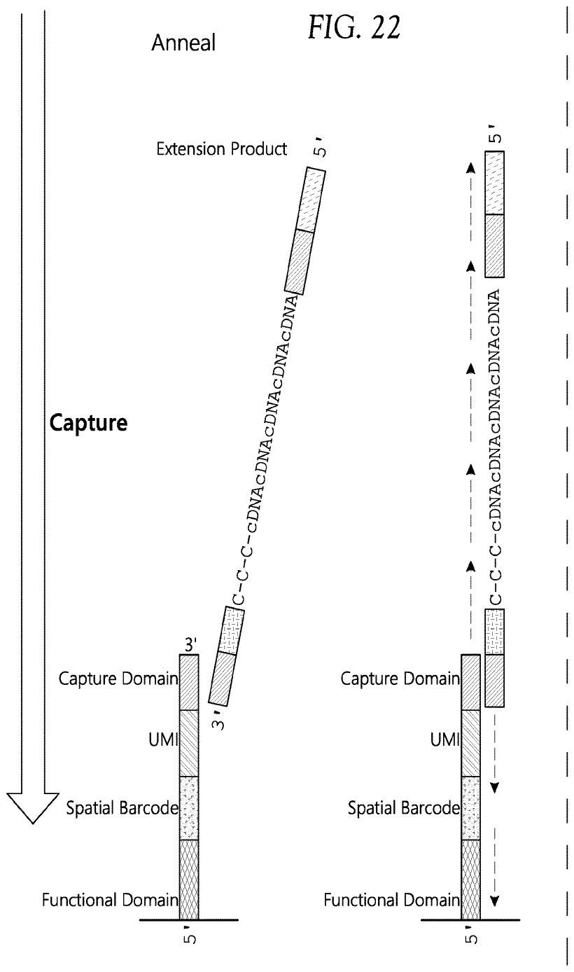

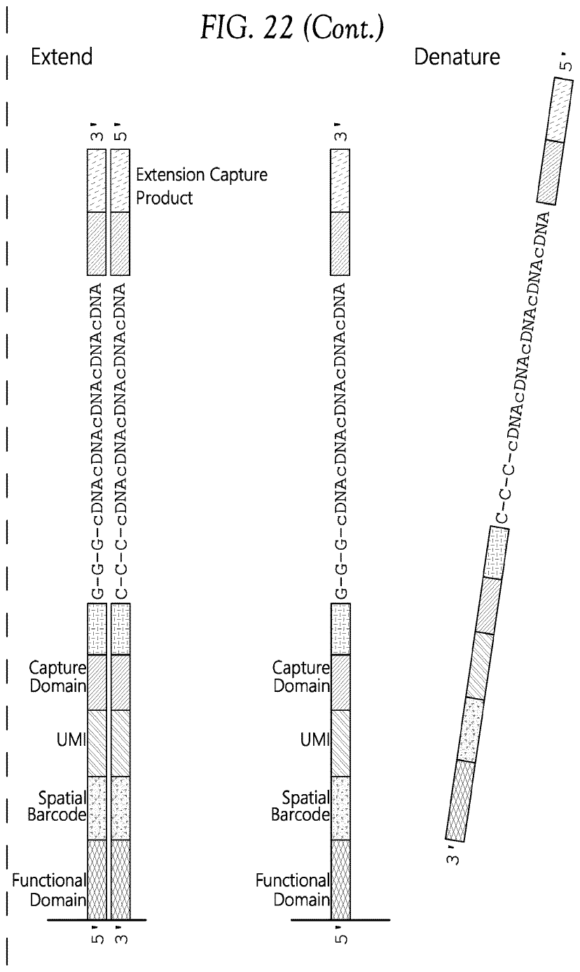

FIG. 22 is a schematic showing capture of the extension product (e.g., the single-stranded cDNA product shown in FIG. 21) by a capture probe on the substrate. The capture probe is attached to the substrate via its 5′ end and can include one or more functional domains, a spatial barcode, a unique molecular identifier, and a capture domain. In some examples, the capture probe also includes a cleavage domain. The capture domain hybridizes to the capture sequence on the extension product (e.g., single-stranded cDNA product) from FIG. 21. In some examples, the 3′ end of the capture probe is extended using the extension product as a template. In some examples, the 3′ end of the extension product (e.g., single-stranded cDNA product) is extended using the capture probe as a template thereby generating an extended capture product. In some examples, the 3′ end of the capture probe is extended using the extension product as a template and the 3′ end of the extension product is simultaneously extended using the capture probe as a template (e.g., generating an extended capture product). In some examples, the extended capture product is released from the capture probe. In some examples, the extended capture product is released via heat. In some examples, the extended capture product is denatured from the capture probe. In some examples, the extended capture product is denatured from the capture probe with KOH.

The released, extended captured products can be prepared for downstream applications, such as generation of a sequencing library and next-generation sequencing.

### 5′ Capture of Target Nucleic Acids

Since capture on a spatial array generally biases the 3′ end of nucleic acid analytes, methods are needed to determine the sequence of the 5′ end of nucleic acid analytes (e.g., by capturing the 5′ end of the nucleic acid analyte), or a complement thereof (e.g., a proxy of the analyte). For example, target nucleic acid analytes (e.g., RNA) can be reverse transcribed with a first primer including a sequence complementary to the target nucleic acid and a functional domain, such as a primer binding site or a sequencing specific site to generate an RNA/DNA (e.g., cDNA) duplex. An enzyme such as a reverse transcriptase or terminal transferase can add non-templated nucleotides to the 3′ end of the cDNA. For example, a reverse transcriptase or terminal transferase enzyme can add at least 3 nucleotides (e.g., a polynucleotide sequence (e.g., a heteropolynucleotide sequence (e.g., CGC), a homopolynucleotide sequence (e.g., CCC))) to the 3′ end of the cDNA. A second primer that includes a sequence complementary to the non-templated nucleotides (e.g., the polynucleotide sequence) and a capture sequence can hybridize to the non-templated nucleotides (e.g., the polynucleotide sequence) added to the end of the cDNA. In some embodiments, the second primer includes an RNA sequence (e.g., one or more ribonucleotides). The cDNA is extended using the second primer as a template thereby incorporating the complement of the capture sequence into the cDNA. The complement of the capture sequence can hybridize to the capture domain of the capture probe on the substrate. The target nucleic acid with the ribo-second primer can be removed (e.g., digested, denatured, etc.) resulting in a single-stranded DNA product. The single-stranded DNA product can include the functional domain at its 5′ end, a copy of the target analyte (e.g., cDNA), and a complement of the capture sequence that is capable of binding (e.g., hybridizing) to a capture domain of a capture probe on the array at its 3′ end.

Target nucleic acid analytes (also referred to herein as analytes or target nucleic acids) can include a nucleic acid molecule with a nucleic acid sequence encoding at least a portion of a V-J sequence or a V(D)J sequence of an immune cell receptor (e.g., a T cell receptor or a B cell receptor). Target nucleic acids can include a nucleic acid molecule with a nucleic acid sequence encoding an antibody. In some embodiments, the target nucleic acid is RNA. In some embodiments, the RNA is mRNA. In some embodiments, the target nucleic acids are nucleic acids encoding immune cell receptors. In some embodiments, target nucleic acids encoding immune cell receptors identify clonotype populations from a biological sample. In some embodiments, target nucleic acids include a constant region, such as a sequence encoding a constant region of an immune cell receptor (e.g., antibody). In some embodiments, target nucleic acids include a variable region, such as a sequence encoding a variable region of an immune cell receptor (e.g., antibody).

In some embodiments, the target nucleic acid encodes an immune cell receptor. In some embodiments, the immune cell receptor is a B cell receptor. In some embodiments, the B cell receptor includes an immunoglobulin kappa light chain. In some embodiments, the target nucleic acid includes a sequence encoding a CDR3 region of the immunoglobulin kappa light chain. In some embodiments, the target nucleic acid includes a sequence encoding one or both of CDR1 and CDR2 of the immunoglobulin kappa light chain. In some embodiments, the target nucleic acid includes a sequence encoding a full-length variable domain of the immunoglobulin kappa light chain.

In some embodiments, the B cell receptor includes an immunoglobulin lambda light chain. In some embodiments, the target nucleic acid includes a sequence encoding a CDR3 of the immunoglobulin lambda light chain. In some embodiments, the target nucleic acid includes a sequence encoding one or both of CDR1 and CDR2 of the immunoglobulin lambda light chain. In some embodiments, the target nucleic acid includes a sequence encoding a full-length variable domain of the immunoglobulin lambda light chain.

In some embodiments, the B cell receptor includes an immunoglobulin heavy chain. In some embodiments, the target nucleic acid includes a sequence encoding a CDR3 of the immunoglobulin heavy chain. In some embodiments, the target nucleic acid includes a sequence encoding one or both of CDR1 and CDR2 of the immunoglobulin heavy chain. In some embodiments, the target nucleic acid includes a sequence encoding a full-length variable domain of the immunoglobulin heavy chain.

In some embodiments, the immune cell receptor is a T cell receptor. In some embodiments, the T cell receptor includes a T cell receptor alpha chain. In some embodiments, the target nucleic acid includes a sequence encoding a CDR3 of the T cell receptor alpha chain. In some embodiments, the target nucleic acid includes a sequence encoding one or both of CDR1 and CDR2 of the T cell receptor alpha chain. In some embodiments, the target nucleic acid includes a sequence encoding a full-length variable domain of the T cell receptor alpha chain.

In some embodiments, the T cell receptor includes a T cell receptor beta chain. In some embodiments, the target nucleic acid includes a sequence encoding a CDR3 of the T cell receptor beta chain. In some embodiments, the target nucleic acid includes one or both of CDR1 and CDR2 of the T cell receptor beta chain. In some embodiments, the target nucleic acid further includes a full-length variable domain of the T cell receptor beta chain.

Provided herein are methods for determining a location of a target nucleic acid in a biological sample, the method including: (a) contacting the biological sample with a first primer including a nucleic acid sequence that hybridizes to a complementary sequence in the target nucleic acid and a functional domain; (b) hybridizing the first primer to the target nucleic acid and extending the first primer using the target nucleic acid as a template to generate an extension product; (c) adding a non-templated sequence (e.g., a polynucleotide sequence including at least three nucleotides) to the 3′ end of the extension product; (d) hybridizing a second primer to the non-templated sequence (e.g., polynucleotide sequence comprising at least three nucleotides of the extension product of (c)), where the second primer comprises a capture sequence; (e) extending the extension product using the second primer as a template, thereby incorporating a complement of the capture sequence into the extension product; (f) hybridizing the complement of the capture sequence of the extension product to a capture domain on an array, wherein the array includes a plurality of capture probes, and wherein a capture probe of the plurality of capture probes comprises a spatial barcode and the capture domain; and (g) determining (i) the sequence of the spatial barcode, or a complement thereof, and (ii) all or a portion of the sequence of the target nucleic acid, or a complement thereof, and using the determined sequences of (i) and (ii) to determine the location of the target nucleic acid in the biological sample.

The methods described herein can also use a plurality of primers, wherein the first primer is comprised in the plurality of primers. For example, the plurality of primers can hybridize to a target nucleic acid at different locations in the target nucleic acid and subsequently be extended. Extending the plurality of primers generates one or more extension products that include a complement of the capture sequence as described herein. For example, the methods provided herein can include providing a plurality of primers wherein each primer includes a sequence that hybridizes to a complementary sequence in the target nucleic acid and a functional domain, wherein the first primer is comprised in the plurality of primers and (a) hybridizing the plurality of primers to the target nucleic acid and extending one or more primers from the plurality of primers using the target nucleic acid as a template to generate one or more extension products; (b) attaching a non-templated sequence (e.g., a polynucleotide sequence including at least three nucleotides) to the 3′ end of the one or more extension products; (c) hybridizing the second primer to the non-templated sequence (e.g., polynucleotide sequence) of the one or more extension products of (b), where the second primer includes a capture sequence; (d) extending the one or more extension products using the second primer as a template, thereby incorporating a complement of the capture sequence into the one or more extension products; (e) hybridizing the complement of the capture sequence of the one or more extension products to a capture domain on an array, where the array includes a plurality of capture probes, and where a capture probe of the plurality of capture probes comprises a spatial barcode and the capture domain; and (f) determining (i) the sequence of the spatial barcode, or a complement thereof, and (ii) all or a portion of the sequence of the target nucleic acid, or a complement thereof, and using the determined sequences of (i) and (ii) to determine the location of the target nucleic acid in the biological sample.

### Compositions of the Disclosure

One aspect of the present disclosure relates to antigen-binding molecules (e.g., antibodies or TCRs) or antigen-binding fragments thereof that were identified by a method disclosed herein. There are no particular limitations to the types of antigen-binding molecules or antigen-binding fragments thereof that can be suitably identified by the methods disclosed herein. Examples of suitable antigen-binding molecules include, but are not limited to, those capable of binding or as having an affinity for a target antigen associated with an infectious agent, such as a viral agent, bacterial agent, parasitic agent, protozoal agent, or prion agent. Further, the target antigen may be associated with a tumor or a tumor or cancer. In addition, the target antigen may be an immune checkpoint molecule that may or may not be associated with tumors or cancers, or it may be a cytokine, a GPCR, a cell-based co-stimulatory molecule, a cell-based co-inhibitory molecule, an ion channel, or a growth factor. Further still, the target antigen may be associated with a degenerative condition or disease (e.g., an amyloid protein).

Also provided are nucleic acids encoding the antigen-binding molecules (e.g., antibodies or TCRs) and antigen-binding fragments as disclosed herein, recombinant cells and transgenic animals engineered to produce the antibodies and antigen-binding fragments as disclosed herein, and pharmaceutical compositions containing one or more of the nucleic acids, recombinant cells, and antibodies and antigen-binding fragments as disclosed herein.

### Antigen-Binding Molecules

One aspect of the present disclosure relates to antigen-binding polypeptides that were identified by a method disclosed herein, such as antibodies and antigen-binding fragments thereof, e.g., that specifically bind to a target antigen.

An antibody is generally understood by the skilled artisan in the art to refer to immunoglobulin molecules including four polypeptide chains, two heavy chains (HCs) and two light chains (LCs) inter-connected by disulfide bonds (i.e., “full antibody molecules”), as well as multimers thereof (e.g. IgM). Each heavy chain includes a heavy chain variable region (“HCVR” or “VH”) and a heavy chain constant region (which is comprised of domains CH1, CH2 and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FWR). Each VH and VL includes three CDRs and four FWRs, arranged from amino-terminus to carboxy-terminus in the following order: FWR1, CDR1, FWR2, CDR2, FWR3, CDR3, and FWR4. Heavy chain CDRs can also be referred to as HCDRs, and numbered as described above (e.g., HCDR1, HCDR2, and HCDR3). Likewise, light chain CDRs can be referred to as LCDRs, and numbered LCDR1, LCDR2, and LCDR3. In some embodiments of the disclosure, the FRs of the antibodies or antigen binding fragments thereof are identical to the human germline sequences, or are naturally or artificially modified.

In some embodiments of the disclosure, the assignment of amino acids to each domain is in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

The term “antigen-binding fragment” of an antibody or antigen-binding polypeptide, as used herein, include any naturally occurring, enzymatically obtainable, synthetic, or genetically engineered polypeptide or glycoprotein that specifically binds an antigen to form a complex. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody, in some embodiment of the disclosure, include at least one variable domain. The variable domain can be of any size or amino acid composition and will generally include at least one CDR, which is adjacent to or in frame with one or more framework sequences. In antigen-binding fragments having a VH domain associated with a VL domain, the VH and VL domains can be situated relative to one another in any suitable arrangement. For example, the variable region can be dimeric and contain VH-VH, VH-VL or VL-VL dimers. Alternatively, the antigen-binding fragment of an antibody can contain a monomeric VH or VL domain.

In some embodiments, an antigen-binding fragment of an antibody can contain at least one variable domain covalently linked to at least one constant domain. Non-limiting, exemplary configurations of variable and constant domains that can be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains can be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may include a homo-dimer or hetero-dimer (or other multimer) of any of the variable and constant domain configurations listed above in non-covalent association with one another and/or with one or more monomeric VH or VL domain (e.g., by disulfide bond(s)). Antigen-binding proteins (e.g., antibodies and antigen-binding fragments) can be mono-specific or multi-specific (e.g., bi-specific).

In some embodiments, the antibody or antigen-binding fragment of the disclosure further includes a constant region. In some embodiments, the constant region is an IgA, IgD, IgE, IgG, or IgM heavy chain constant region. In some embodiments, the antibody or antigen-binding fragment of the disclosure includes a constant region of the type IgA (e.g., IgA1 or IgA2), IgD, IgE, IgG (e.g., IgG1, IgG2, IgG3 and IgG4) or IgM. In some embodiments, the constant region is an IgG constant region.

In some embodiments, the antibody or antigen-binding fragment of the disclosure further includes a kappa type light chain constant region. In some embodiments, the antibody or antigen-binding fragment of the disclosure further includes a lambda type light chain constant region.

In some embodiments, the antibody or antigen-binding fragment of the disclosure is a human antibody or antigen-binding fragment. One of ordinary skill in the art will understand that the term “human” antibody includes antibodies having variable and constant regions derived from human germline immunoglobulin sequences whether in a human cell or grafted into a non-human cell, e.g., a mouse cell. The human antibodies of the disclosure can include amino acid residues not encoded by human germline immunoglobulin sequences (e.g., mutations introduced by random or site-specific mutagenesis in vitro or by somatic mutation in vivo), for example in the CDRs, such as CDR3. However, the term “human antibody,” as used herein, is not intended to include antibodies in which CDR sequences derived from the germline of another mammalian species (e.g., mouse) have been grafted onto human FWR sequences. The term includes antibodies recombinantly produced in a non-human mammal or in cells of a non-human mammal.

In some embodiments, the antibody or antigen-binding fragment is a humanized antibody, a chimeric antibody, or a hybrid antibody. The term “humanized antibody” as used herein encompasses antibodies comprising heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody in which human CDR sequences are introduced into non-human VH and VL sequences to replace the corresponding non-human CDR sequences. Another type of humanized antibody is a FWR-grafted antibody in which human FWR sequences are introduced into non-human VH and VL sequences to replace corresponding non-human FWR sequences. In some embodiments, the antibodies or antigen-binding fragments of the disclosure include a murine antibody, phage display antibody, or nanobody/VHH containing the frameworks and/or CDRs described in this disclosure. As used herein, the term “chimeric antibody” encompasses antibodies having the variable domain from a first antibody and the constant domain from a second antibody, wherein the first and second antibodies are from different species. As used herein, the term “hybrid antibody” encompasses antibodies having the variable domain from a first antibody and the constant domain from a second antibody, wherein the first and second antibodies are from different animals, or wherein the variable domain, but not the constant region, is from a first animal. For example, a variable domain can be taken from an antibody isolated from a human and expressed with a fixed constant region not isolated from that antibody. In some embodiments, hybrid antibodies can be synthetic and/or non-naturally occurring because the variable and constant regions they contain are not isolated from a single natural source. In some embodiments, the hybrid antibodies of the disclosure includes a light chain from a first antibody and a heavy chain from a second antibody, wherein the first and second antibodies are from different species. In some embodiments, the chimeric antibodies of the disclosure includes a non-human light chain which is combined with a heavy chain or set of heavy chain CDRs disclosed in this application.

In some embodiments, the antibody is a monoclonal antibody. In some embodiments, the antibody or antigen-binding fragment is a single-chain antibody fragment (scFv), a Fab, a Fab′, a Fab′-SH, a F(ab′)2, or a Fv fragment.

In some embodiments, the antibodies and antigen-binding fragments of the disclosure have a neutralizing activity (e.g., antagonistic activity) against the target antigen (e.g., SARS-CoV-2), e.g., able to bind to and neutralize the activity of the antigen (e.g., SARS-CoV-S), as determined by in vitro or in vivo assays. The ability of the antibodies of the disclosure to bind to, block and/or neutralize the activity of the target antigen (e.g., SARS-CoV-2) may be measured using any standard method known to those skilled in the art, including binding assays, or activity assays, as described herein.

### Nucleic Acids

As discussed above, one aspect of the disclosure relates to recombinant nucleic acids including a nucleic acid sequence that encodes an ABM (e.g., antibody or TCR) of the disclosure or an antigen-binding fragment thereof. In some embodiments, the recombinant nucleic acids of the disclosure can be configured as expression cassettes or vectors containing these nucleic acid molecules operably linked to heterologous nucleic acid sequences such as, for example, regulatory sequences which allow in vivo expression of the receptor in a host cell.

Accordingly, in some embodiments, provided herein is a nucleic acid molecule including a nucleotide sequence encoding an ABM (e.g., antibody, TCR) of the disclosure or an antigen-binding fragment thereof. In some embodiments, the nucleotide sequence is incorporated into an expression cassette or an expression vector. It will be understood by the skilled artisan that an expression cassette generally includes a construct of genetic material that contains coding sequences of the antibody or antigen-binding fragment thereof and enough regulatory information to direct proper transcription and/or translation of the coding sequences in a recipient cell, in vivo and/or ex vivo. Generally, the expression cassette can be inserted into a vector for targeting to a desired host cell and/or into an individual. As such, in some embodiments, an expression cassette of the disclosure include a coding sequence for an antibody of the disclosure or an antigen-binding fragment thereof, which is operably linked to expression control elements, such as a promoter, and optionally, any or a combination of other nucleic acid sequences that affect the transcription or translation of the coding sequence.

An expression cassette can be inserted into a plasmid, cosmid, virus, autonomously replicating polynucleotide molecule, phage, as a linear or circular, single-stranded or double-stranded, DNA or RNA polynucleotide molecule, derived from any source, capable of genomic integration or autonomous replication, including a nucleic acid molecule where one or more nucleic acid sequences has been linked in a functionally operative manner, e.g., operably linked.

The nucleic acid sequences encoding the antibodies and antigen-binding fragments as disclosed herein can be optimized for expression in the host cell of interest. For example, the G-C content of the sequence can be adjusted to average levels for a given cellular host, as calculated by reference to known genes expressed in the host cell. Methods for codon usage optimization are known in the art.

Also provided herein are vectors, plasmids, or viruses containing one or more of the nucleic acid molecules encoding any antibody or an antigen-binding fragment thereof as disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan. See for example, Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference).

### Recombinant Cells

The nucleic acid of the present disclosure can be introduced into a host cell, such as, for example, a Chinese hamster ovary (CHO) cell, to produce a recombinant cell containing the nucleic acid molecule. Introduction of the nucleic acid molecules (e.g., DNA or RNA, including mRNA) or vectors of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery. Accordingly, in some embodiments, the nucleic acid molecules can be delivered by viral or non-viral delivery vehicles known in the art.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the animal cell is a mammalian cell. In some embodiments, the animal cell is a human cell. In some embodiments, the animal cell is a non-human animal cell. In some embodiments, the cell is a non-human primate cell. In some embodiments, the recombinant cell is selected from the group consisting of a baby hamster kidney (BHK) cell, a Chinese hamster ovary cell (CHO cell), an African green monkey kidney cell (Vero cell), a human A549 cell, a human cervix cell, a human CHME5 cell, a human PER.C6 cell, a NS0 murine myeloma cell, a human epidermoid larynx cell, a human fibroblast cell, a human HEK-293 cell, a human HeLa cell, a human HepG2 cell, a human HUH-7 cell, a human MRC-5 cell, a human muscle cell, a mouse 3T3 cell, a mouse connective tissue cell, a mouse muscle cell, and a rabbit kidney cell. In some embodiments, the recombinant cell is a Pichia pastoris cell or a Saccharomyces cerevisiae cell, both of which are also suitable for production of scFv, scFv-Fe, Fab, and F(ab′)2.

Also provided, in another aspect, are animals including a recombinant nucleic acid or a vector as disclosed herein. In some embodiments, the disclosure provides a transgenic animal that is a non-human animal. In some embodiments, the transgenic animal produces an antibody or antigen-binding fragment as disclosed herein. In some embodiments, the animal is a vertebrate animal or an invertebrate animal. In some embodiments, the animal is a mammalian subject.

### Pharmaceutical Compositions

The ABMs, such as TCRs, antibodies and/or antigen-binding fragments thereof, nucleic acids, recombinant cells, and/or cell cultures of the disclosure can be incorporated into compositions, including pharmaceutical compositions.

Exemplary compositions of the disclosure include pharmaceutical compositions which generally include one or more of the antibodies, TCRs, or antigen-binding fragments thereof, nucleic acids, recombinant cells, and/or cell cultures as described herein and a pharmaceutically acceptable excipient, e.g., carrier. In some embodiments, the composition is a sterile composition. In some embodiments, the composition is formulated as a vaccine. In some embodiments, the composition further includes an adjuvant.

The pharmaceutical compositions provided herein can be in any form that allows for the composition to be administered to an individual. In some specific embodiments, the pharmaceutical compositions are suitable for human administration. The scope of the present disclosure includes desiccated, e.g., freeze-dried, compositions comprising an anti-CoV-S antigen-binding polypeptides, e.g., antibody or antigen-binding fragment thereof, or a pharmaceutical composition thereof that includes a pharmaceutically acceptable carrier but substantially lacks water. As used herein, the term “pharmaceutically acceptable” means approved by a regulatory agency of the Federal or a state government or listed in the U.S. Pharmacopeia or other generally recognized pharmacopeiae for use in animals, and more particularly in humans. The carrier can be a diluent, adjuvant, excipient, or vehicle with which the pharmaceutical composition is administered. Saline solutions and aqueous dextrose and glycerol solutions can also be employed as liquid carriers, including injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. In some embodiments, the pharmaceutical composition is sterilely formulated for administration into an individual or an animal (some non-limiting examples include a human, or a mammal). In some embodiments, the individual is a human.

In some embodiments, the pharmaceutical compositions of the present disclosure are formulated to be suitable for the intended route of administration to an individual. For example, the pharmaceutical composition can be formulated to be suitable for parenteral, intraperitoneal, colorectal, intraperitoneal, and intratumoral administration. In some embodiments, the pharmaceutical composition can be formulated for oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal or intra-arterial administration. One of ordinary skilled in the art will appreciate that the formulation should suit the mode of administration.

### Kits

Further provided herein are kits for the practice of a method described herein. In some embodiments, provided herein are kits for identification or characterization of antibodies, TCRs, and antigen-binding fragments thereof having binding affinity for an antigen. Such kits can include a plurality of target antigens and non-target antigens, and instructions for performing a method of the disclosure. The kits can also include a spatial array containing capture probes.

In particular embodiments, a kit for identifying and/or characterizing an ABM or fragment thereof having binding affinity for an antigen includes: (a) a plurality of target antigens and non-target antigens, wherein each of the target antigens and non-target antigens comprise a reporter oligonucleotide comprising (i) a barcode sequence that identifies the antigen or non-antigen, and (ii) a capture handle sequence; (b) a spatial array comprising a first capture probe comprising (i) a barcode sequence, and (ii) a first capture domain, and a second capture probe comprising (i) a barcode sequence, and (ii) a second capture domain. In some embodiments, the kit further includes instructions for performing a method disclosed herein.

Also provided, in some embodiments of the disclosure, are kits for producing an ABM or antigen-binding fragment thereof, (ii) detecting the presence of an antigen in a biological sample, or (iii) treating, preventing, or ameliorating a health condition associated with an antigen in a subject.

A kit can include instructions for use thereof and one or more of the ABMs or antigen-binding fragments thereof, recombinant nucleic acids, recombinant cells, and pharmaceutical compositions as described and provided herein. For example, some embodiments of the disclosure provide kits that include one or more of the antibodies described herein and/or antigen-binding fragments thereof, and instructions for use. In some embodiments, provided herein are kits that include one or more recombinant nucleic acids, recombinant cells, and pharmaceutical compositions as described herein and instructions for use thereof.

In some embodiments, the components of a kit can be in separate containers. In some other embodiments, the components of a kit can be combined in a single container.

In some embodiments, a kit can further include instructions for using the components of the kit to practice a method described herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

In some embodiments, a kit can further include reagents and instructions for preparing target antigens for use in accordance with the disclosed methods. In some embodiments, a kit can include reagents and instructions for performing spatial analysis. In some embodiments, a kit can include reagents and instructions for identifying and/or characterizing an ABM or fragment thereof having binding affinity for a target antigen.

The instructions for practicing the method are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

Additional embodiments are disclosed in further detail in the following examples, which are provided by way of illustration and are not in any way intended to limit the scope of this disclosure or the claims.

### EXAMPLES

### Example 1: Determination of the Identity and Spatial Location of an Antigen Specific Antibody in a Tissue Using Barcode-Enabled Antigen Mapping (BEAM-Ab)

This example provides an exemplary method of using barcoded target and non-target antigens to identify and determine the spatial location of an antibody that potentially binds the target antigen.

In a non-limiting example, a target antigen (e.g., a cancer antigen, a SARS-CoV-2 spike protein) and non-target antigen (e.g., Human Serum Albumin: HSA) are biotinylated. In some embodiments, the biotinylation can be performed using Avitag™ technology. For example, the antigen and non-antigen can include a polyhistidine tag at their C-termini followed by an Avi tag, and a single lysine residue in the Avitag is enzymatically labeled with biotin.

The biotinylated antigens (target and non-target) are each solubilized in sterile deionized water (e.g., for 30-60 minutes at room temperature with occasional gentle mixing for a final concentration of 100 microgram/mL (for HSA) or 200 microgram/mL for target antigen). Solubilized, biotinylated antigens are each conjugated with, e.g., allowed to form a complex with (or bind to) a streptavidin-barcoded DNA conjugate. This can be, for example, one of the following TotalSeqC reagents, supplied by BioLegend, which each contain a unique barcoded DNA oligonucleotide (i.e., a reporter oligonucleotide) used as follows:
1) TotalSeq-C0951 PE Streptavidin is conjugated to biotinylated target antigen.
2) TotalSeq-C0952 PE Streptavidin is conjugated to biotinylated human serum albumin.

The final conjugated antigen probes (barcoded streptavidin-biotinylated antigen complexes) are then used for biological sample labeling, e.g., at a dilution of 1:50.

A tissue sample is obtained (e.g., a fresh frozen tissue or fixed tissue, e.g., from a subject having a tumor). The tissue sample can be from a tissue known to be infiltrated by or have high numbers of B-cells, such as, lymph nodes, spleen, femur and tibia. The tissue sample is processed for spatial analysis according to a method disclosed herein. For example, A substrate having a spatial array of capture probes, where each probe includes a spatial barcode and a capture domain is provided (e.g., a Visium Spatial Gene Expression slide by 10× Genomics, as described in the Visium Spatial Gene Expression Reagent Kits User Guide (e.g., Rev D, dated October 2020)). The final conjugated antigen probes are applied to the tissue sample allowing the antigens to bind to a corresponding ABM in the tissue sample. The tissue sample is permeabilized and nucleic acid analytes from cells (e.g., an ABM-expressing cell) within the tissue, as well as the reporter oligonucleotides associated with the target and non-target antigens, are attached (e.g., hybridized) to capture probes, e.g., according to spatial analysis methods known in the art or disclosed herein (for example, the tissue sample can be contacted with or brought into proximity to the array of capture probes and the analytes/barcode reporter oligonucleotides can be released to the capture probes). The captured nucleic acid analytes and barcode reporter oligonucleotides are used in extension reactions to produce spatially barcoded extension products including sequences corresponding to the captured nucleic acid analytes and/or reporter oligonucleotides, respectively. The spatially barcoded extension products are used to produce gene expression and reporter oligonucleotide libraries.

V(D)J, barcoded antigen, and optionally standard gene expression libraries, are then constructed using e.g., spatial analysis methods known in the art or disclosed herein. The barcoded antigen library can include spatially barcoded polynucleotides or amplicons or library members thereof including (i) the barcode sequence of the reporter oligonucleotide or reverse complement thereof and (ii) a spatial barcode sequence or reverse complement thereof. The V(D)J library can include native sequences of antibodies or fragments thereof in the tissue or a reverse complement thereof and spatial barcode sequence or reverse complement thereof.

The generated libraries are sequenced. Sequencing is performed by various available systems, such as, without limitation, a sequencing system by Illumina®, Pacific Biosciences (PacBio®), Oxford Nanopore®, or Life Technologies (Ion Torrent®). Sequence analysis is used to identify antibodies expressed in the tissue as well as to determine the spatial location of the antibodies in the tissue. Analysis of the barcode antigen library also indicates spatial locations where the target and/or non-target antigens bind to the tissue.

In some embodiments, the binding affinity of an ABM (e.g., antibody) to a target antigen (e.g., cancer antigen) is determined based on quantity/numbers of unique molecular identifiers (UMIs) associated with the target antigen bound by or proximal to the ABM or associated with the ABM bound or proximal to the target antigen. Generally, an identified antibody displaying high target antigen counts and low non-target antigen counts is predicted to have specific binding to/affinity for the target antigen and is distinguishable from non-specific binders.

### Example 2: Determination of the Identity and Spatial Location of an Antigen Specific TCR in a Tissue Using Barcode-Enabled Antigen Mapping (BEAM-T)

This example provides an exemplary method of using barcoded MHC-target (and non-target) antigen multimers to identify and determine the spatial location of a T-cell receptor that potentially binds the target antigen.

Many TCRs can bind a particular antigen (with varying affinity) and identifying individual clonotypes specific to a particular antigen is difficult. While flow cytometry and bead-based enrichment schemes allow physical sorting of antigen-binding cells, when cells are rare or samples are limited, cell losses associated with traditional methodologies can be unacceptable. Moreover, traditional approaches based on fluorescent detection have important limitations with regard to multiplexing (the ability to simultaneously assay the binding properties of multiple independent antigens/ligands in single experiment) due to the small number of spectrally distinguishable fluorescent labels that can be effectively used in combination. Furthermore, multiple antigen-binding clonotypes may be present in a heterogeneous sample, which makes identifying specific antigen-binding TCR complexes difficult, even when the cells expressing antigen-binding clonotypes are physically sorted.

The compositions, methods, and systems described herein allow functionalization of MHC-peptide multimers with an oligonucleotide (DNA or RNA) that includes a unique peptide barcode sequence specific to the MHC-peptide identity (e.g., Barcode 1 associated with peptide EGALIYWPN, Barcode 2 associated with peptide AHMRDSQQ, etc.). A single peptide-MHC complex or peptide-MHC library can be exposed to a biological sample such as a tissue sample to produce the sample tagged with barcoded MHC multimers. These sample can then be partitioned and processed as described herein to assemble TCR sequences and quantify the number of MHC-peptide barcodes associated therewith. Clonotypes with low levels of MHC-peptide derived UMIs have a low affinity for the MHC-peptide while clonotypes with high levels of the MHC-peptide UMIs have a high affinity for the antigen.

Barcoded, peptide-bound MHC tetramers bound to a streptavidin core are generated as described below. Although Class I MHC-tetramers are utilized in the following experiments, there are many possible configurations of Class I and/or Class II MHC-antigen multimers that can be utilized with the compositions, methods, and systems disclosed herein, e.g., MHC pentamers (MHC assembled via a coiled-coil domain, e.g., Pro5® MHC Class I Pentamers, (ProImmune, Ltd.), MHC decorated dextran molecules (e.g., MHC Dextramer® (Immudex)), etc.

Streptavidin molecules are conjugated to a hybridization oligonucleotide e.g., using general lysine chemistry (streptavidin modified via lysine residues with NHS-DBCO; subsequently an azide-modified oligonucleotide is attached via the DBCO functional group) to produce streptavidin-conjugated oligonucleotides. Streptavidin-conjugated oligonucleotides (can be analyzed on a TBE-urea denaturing agarose gel. Relative to unmodified streptavidin or oligonucleotides, streptavidin-conjugated oligonucleotides exhibit a molecular weight shift indicative of streptavidin conjugated with 0, 1, 2, 3, 4 (or more) oligonucleotides.

Barcode reporter oligonucleotides are hybridized to the streptavidin-conjugated oligonucleotides based on sequence complementarity. In some embodiments, the barcode reporter oligonucleotides include a sequence that is the reverse complement of the hybridization oligo sequence (sometimes referred to as “anchor” sequence), a barcode sequence, a capture handle sequence, and optionally, a functional sequence (e.g., a TruSeq R2 sequencing primer sequence, or adapter sequence). Alternatively, the barcode reporter oligonucleotide can is directly conjugated to the streptavidin.

The barcoded streptavidin is added to a pool of biotinylated HLA-A-02:01 MHC monomers displaying a target peptide antigen (e.g., a cancer antigen, a SARS-CoV-2 spike protein) or non-target peptide to produce barcoded MHC tetramers. The barcoded streptavidin is added until a 1:1 ratio of biotinylated peptide-MHC monomers to biotin binding sites is achieved (4 biotinylated peptide-MHC monomers/streptavidin complex).

Barcoded MHC tetramers are then incubated with a tissue sample allowing the peptide antigen-MHC complexes to bind to a corresponding ABM (e.g., TCR) in the tissue. The tissue sample can be from a tissue known to be infiltrated by or have high numbers of T-cells, such as, infected tissue, cancer tissue. The tissue sample can be contacted with a substrate having a spatial array of capture probes, where each probe includes a spatial barcode and a capture domain (e.g., a Visium Spatial Gene Expression slide by 10× Genomics, as described in the Visium Spatial Gene Expression Reagent Kits User Guide (e.g., Rev D, dated October 2020)). The tissue sample is permeabilized and nucleic acid analytes from cells (e.g., a T-cell) within the tissue, as well as the barcode reporter oligonucleotides associated with the target and non-target peptide-MHC complexes, are attached (e.g., hybridized) to capture probes, e.g., according to spatial analysis methods known in the art or disclosed herein. The captured nucleic acid analytes and barcode reporter oligonucleotides are used in extension reactions to produce spatially barcoded extension products including sequences corresponding to the captured nucleic acid analytes and/or reporter oligonucleotides, respectively. The spatially barcoded extension products are used to produce gene expression and reporter oligonucleotide libraries.

For example, V(D)J, barcoded antigen, and optionally standard gene expression libraries, are then constructed using e.g., spatial analysis methods known in the art or disclosed herein. The barcoded antigen library can include spatially barcoded polynucleotides or amplicons or library members thereof including (i) the barcode sequence of the reporter oligonucleotide or reverse complement thereof and (ii) a spatial barcode sequence or reverse complement thereof. The V(D)J library can include native sequences of T-cell receptors or fragments thereof in the tissue or a reverse complement thereof and spatial barcode sequence or reverse complement thereof.

The generated libraries are sequenced. Sequencing is performed by various available systems, such as, without limitation, a sequencing system by Illumina®, Pacific Biosciences (PacBio®), Oxford Nanopore®, or Life Technologies (Ion Torrent®). Sequence analysis is used to identify T-cell receptors (TCR) expressed in the tissue, as well as to determine the spatial location of the TCR in the tissue. Analysis of the barcode antigen library also indicates spatial locations where the target and/or non-target antigens bind to the tissue. T-cell receptor clonotypes are assembled bioinformatically and the number of UMI counts from barcoded MHC tetramers are quantified per clonotype.

In some embodiments, the binding affinity of an ABM (e.g., TCR) to a target antigen (e.g., cancer antigen) is determined based on quantity/numbers of unique molecular identifiers (UMIs) associated with the target antigen bound by or proximal to the ABM or associated with the ABM bound or proximal to the target antigen. Generally, an identified TCR displaying high target antigen counts and low non-target antigen counts is predicted to have specific binding to/affinity for the target antigen and is distinguishable from non-specific binders.

### Example 3: Combinatorial Profiling of Antigen Panels and Immune Cell Features

A tissue sample is stained with a barcoded antigen panel (e.g., see Example 1) and a panel of barcoded antibodies for profiling immune cells, including antibodies from a “T and B Natural Killer” (TBNK) panel with binding affinity for individual immune cell features such as CD3, CD4 and CD8 (for T-cells), CD56 (for NK cells), and CD19 (for B-cells). In this experiment, each barcoded antibody of the panel of barcoded antibodies includes a reporter oligonucleotide that identifies the immune cell feature. Separate sequencing handles are deployed for the TBNK panel and the antigen panel to facilitate various downstream applications, including identification and/or isolation of antibodies that specifically bind a target antigen.

### Example 4: Recombinant Antibody or TCR Synthesis, Cloning, Expression, and Purification

Using the BCR sequences identified from Example 1, nucleotide sequences encoding variable heavy chain and light chain domains of antibodies are reformatted to IgG1 and synthesized and cloned into a mammalian expression vector. Exemplary mammalian expression vectors are commercially available, e.g., pTwist CMV BG WPRE Neo (Twist Bioscience eCommerce portal), AddGene, InvivoGen, and Human IgG Vector Set from SigmaAldrich. Light chain variable domains are reformatted into kappa and lambda frameworks accordingly. Clonal genes are delivered as purified plasmid DNA ready for transient transfection into suitable cells (e.g., human embryonic kidney (HEK) Expi293 cells (Thermo Scientific), ExpiCHO cells). Cultures in a volume of 1.2 ml are grown to four days, harvested and purified using Protein A resin (PhyNexus) on the Hamilton Microlab STAR platform into 43 mM Citrate 148 mM HEPES, pH 6 to produce a recombinant antibody.

Alternatively, using the TCR sequences identified from Example 2, nucleotide sequences encoding TCR alpha and TCR beta chains are synthesized and cloned into a mammalian expression vector. Clonal genes are delivered as purified plasmid DNA ready for introduction in cultured cells, e.g., Jurkat cells. Such constructs may be introduced via classical transformation techniques, e.g., transfection, transduction, or more precise techniques such as guide RNA (gRNA)-directed CRISPR/Cas genome editing, DNA-guided endonuclease genome editing with NgAgo (Natronobacterium gregoryi Argonaute), or TALENs genome editing (transcription activator-like effector nucleases). Cultures are grown, harvested, and purified to produce a recombinant TCR. A TCR generally includes two polypeptides (e.g., polypeptide chains), such as a α-chain of a TCR, a β-chain of a TCR, a γ-chain of a TCR, a δ-chain of a TCR, or a combination thereof. Several approaches, techniques, and associated reagents for construction of recombinant TCR are known in the art. In some embodiments, the TCR constant region may be further altered to remove one or more domains thereof, which can be achieved by a known genome editing technique (e.g., CRISPR/Cas or TALENs discussed herein), via either homology directed repair, non-homologous end joining (NHEJ), and/or or microhomology-mediated end joining.

In some embodiments, a reporter oligonucleotide containing a reporter barcode sequence is coupled to the recombinant antibody or TCR according to available methods. The reporter barcode sequence is used as an identifier sequence for the antibody or TCR coupled thereto. The recombinant antibody or TCR is used in various downstream analyses.

### Example 5: Further Characterization of Binding Affinity

This Example describes experiments to further characterize binding affinity of select antibodies.

Generally, antibodies predicted to have binding affinity based on antigen UMI count profiles are selected for further screening and analysis. Surface plasmon resonance (SPR) analyses are performed on the selected antibodies by using a Carterra LSA SPR biosensor equipped with a HC30M chip at 25° C. in HBS-TE (10 mM HEPES pH 7.4, 150 mM NaCl, 3 mM EDTA, 0.05% Tween-20). In these experiments, antibodies are diluted to 5 μg/ml in sodium acetate buffer, pH 4.5, and amine-coupled to the sensor chip by EDC/NHS activation, followed by ethanolamine HCl quenching. Increasing concentrations of ligand is flowed over the sensor chip in HBSTE (10 mM HEPES pH 7.4, 150 mM NaCl, 3 mM EDTA, 0.05% Tween-20) with 0.5 mg/ml BSA with 5 minute association and 15 minute dissociation. Following each injection cycle, the surface is regenerated with 2×30 second injections of IgG elution buffer (Thermo).

Traces are analyzed and fit using Carterra's Kinetics Tool software, fit to a 1:1 receptor-ligand binding model. It is observed that the majority of antibodies tested in this study bind to the target antigen used to identify them, as described in Example 1.

To further characterize the antibodies and antigen-binding fragments identified in Example 1, ligand-blocking assays are performed, from which a relative K_{D} value for each mAb can be generated.

In addition, neutralizing activity (e.g., antagonistic activity) of the identified antibodies and antigen-binding fragments is assessed e.g., using live virus or pseudovirus neutralization assays. The assays are performed using the antibodies in a dose-dependent manner to generate an IC₅₀ of neutralization activity. In some experiments, a neutralization activity IC₅₀ value for each antibody is determined in a quantitative focus reduction neutralization test (FRNT) described previously by Zost et al. (Nature, 584:443-449, 2020). In some experiments, neutralization assays are used to determine infectivity of SARS-CoV-2 S protein-containing virus-like particles. In these experiments, a neutralizing or antagonistic CoV-S antibody or antigen-binding fragment can be identified based on its ability to inhibit an activity of CoV-S to any detectable degree, e.g., inhibits or reduces the ability of CoV-S protein to bind to a receptor such as ACE2, to be cleaved by a protease such as TMPRSS2, or to mediate viral entry into a host cell or mediate viral reproduction in a host cell.

### Example 6: Analysis of Antibodies for Binding One or More Antigens Under a Variety of Treatment Conditions

A set of reporter oligonucleotide-associated antigens comprises a first antigen (or epitope) coupled to a first reporter oligonucleotide comprising a first barcode sequence (BC1), and the same first antigen (or epitope) coupled to a second reporter oligonucleotide comprising a second barcode sequence (BC2). In some embodiments, the set of reporter oligonucleotide-associated antigens further includes a second antigen (or epitope) coupled to a third reporter oligonucleotide comprising a third barcode sequence (BC3) and the same second antigen (or epitope) coupled to a fourth reporter oligonucleotide comprising a fourth barcode sequence (BC4). The first antigen may be a target antigen and the second antigen may be a negative control antigen (e.g., as described herein). The BC1-associated first antigen (and optionally the BC3-associated second antigen) is subjected to a first treatment condition (e.g., fixation) and the BC2-associated first antigen (and optionally the BC4-associated second antigen is subjected to a second treatment condition (e.g., no fixation). Thus, the reporter barcode sequences (e.g., BC1-BC4) are used to identify both the antigen (or epitope) and the treatment condition that the antigen (or epitope) is subjected to.

Biological samples (e.g., tissue sample) are contacted with the set of reporter oligonucleotide-associated antigens. Standard gene expression, V(D)J, and barcoded antigen libraries are prepared from the tissue sample as described in Example 1. Sequence analysis of the libraries is used to identify antigen-binding molecules (e.g., antibodies) that bind to antigens subjected to various treatment conditions.

### Example 7: 5′ Capture of Target Nucleic Acids

A fresh frozen mouse brain sample was sectioned and placed on an array slide containing capture probes having a blocked capture domain. The tissue sections were fixed 5 minutes in 4% formaldehyde, followed by 5 minutes of decrosslinking in 0.1N HCl.

The sections were washed in 1×PBS and reverse transcription (RT) was performed using a polydT30NV primer and an in-house reverse transcriptase (RT) enzyme at 42° C. overnight. Fluorescently labeled Cy3-dCTPs were spiked into the RT buffer to permit visualization of the synthesized cDNA. Additionally, a template switching ribonucleotide (rTSO) having a capture sequence as a handle was spiked in to allow the incorporation of the handle into the cDNA.

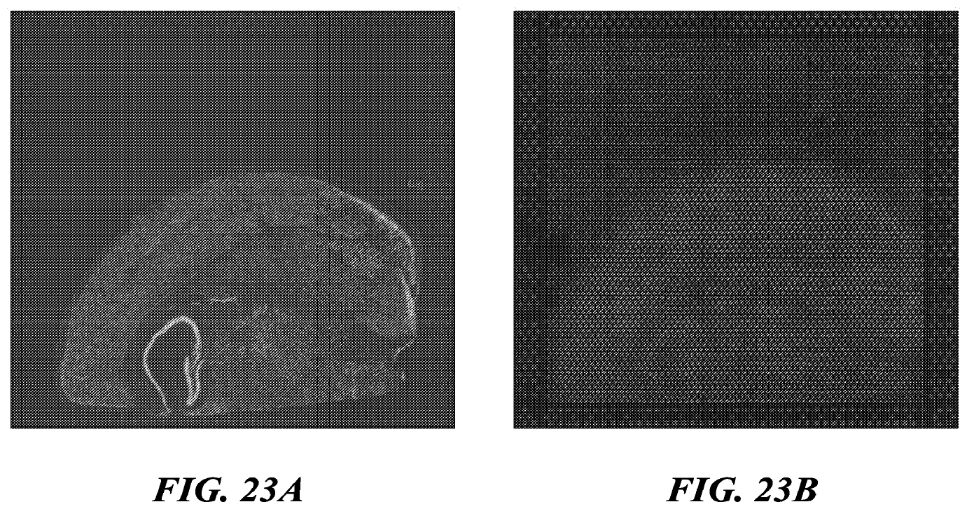

The next day, the sections were washed in 0.2×SSC/20% Ethylene Carbonate at 50° C. to remove any non-specific signal for the array. Afterwards the sections were imaged under the microscope (Cy3 channel, FIG. 23A). Post imaging, the RNA was digested using RNaseH, followed by tissue permeabilization.

Post permeabilization, the bound cDNA was extended using a polymerase and Cy3 was spiked into the mixture. Post extension, the slides were washed in 2×SSC followed by imaging (FIG. 23B).

FIGS. 23A-23B are mouse brain images showing fluorescently labeled cDNA post reverse transcription (FIG. 23A) in situ where the reverse transcription reaction was performed overnight at 42° C. with Cy3 labeled dCTP and results in a tissue “footprint” (e.g., the fluorescently labeled cDNA reproduces the morphological characteristics of the tissue section). FIG. 23B shows fluorescently labeled extended cDNA post permeabilization and cDNA extension which also results in a tissue footprint.

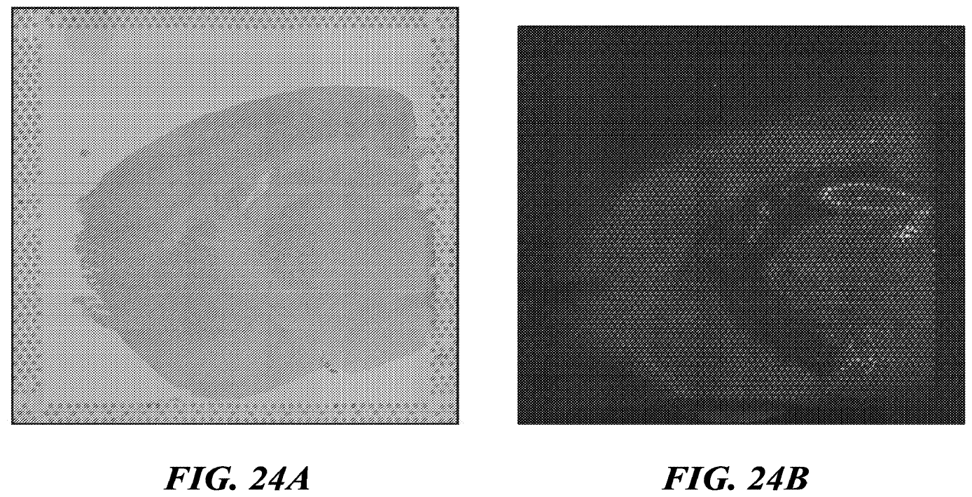

FIGS. 24A-24B show mouse brain images from experiments similar to that described in FIGS. 23A-23B, but the RT reaction was performed without Cy3-dCTP spike in. FIG. 24A shows a brightfield image of a mouse brain tissue section and FIG. 24B shows fluorescently labeled extended cDNA where the capture domain of the capture probe is blocked. During extension, Cy3-dCTPs were spiked in to permit visualization of the captured cDNA.

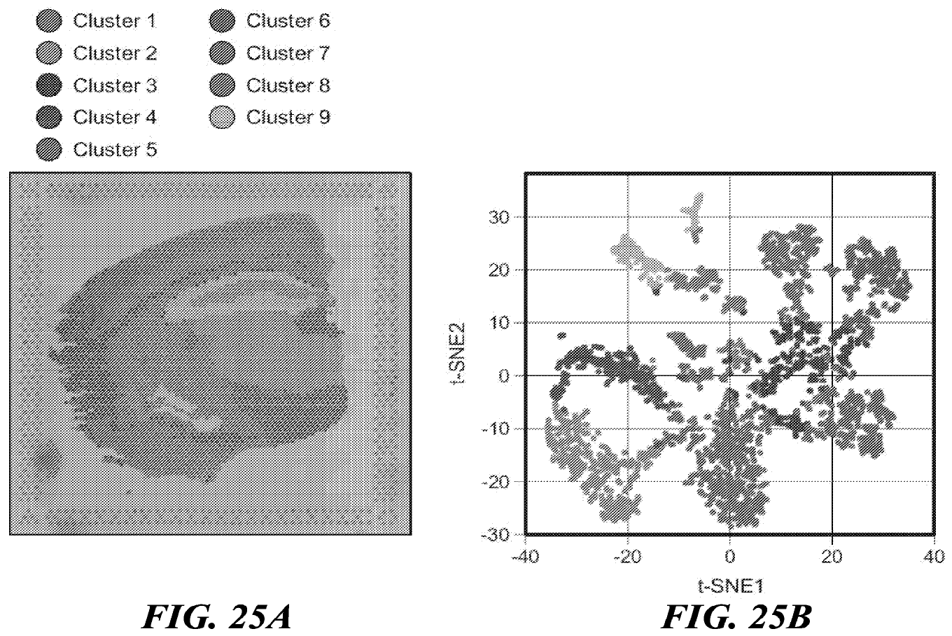

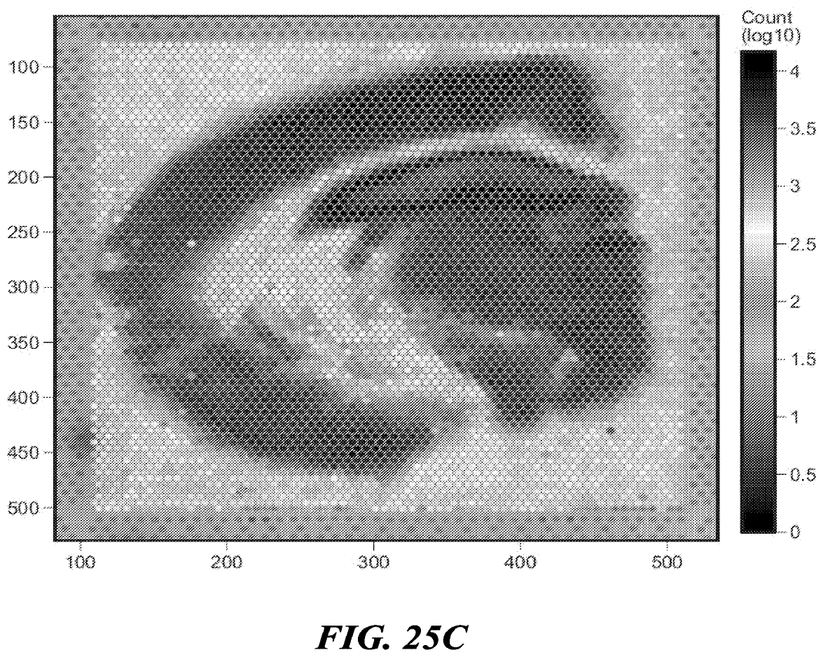

FIG. 25A shows spatial gene expression clusters, the corresponding t-SNE plot (FIG. 25B), and spatial gene expression heat map (FIG. 25C) from capture of extension products generated from experiments as described for FIGS. 23A-23B, except that RT and extension were performed without any Cy3-dCTP spike-in. The captured and extended cDNA was released using 0.08N KOH, followed by standard library preparation for next generation sequencing.

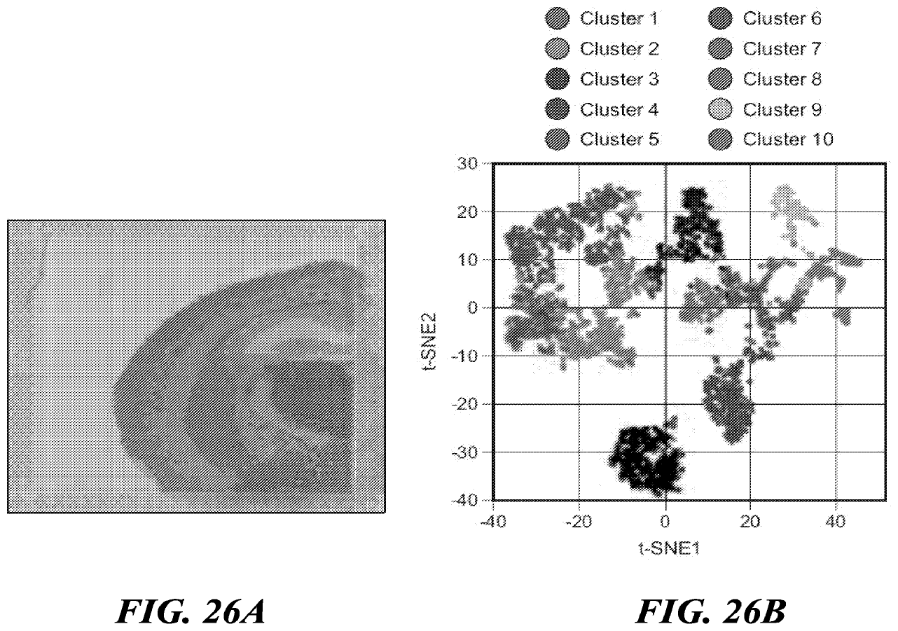

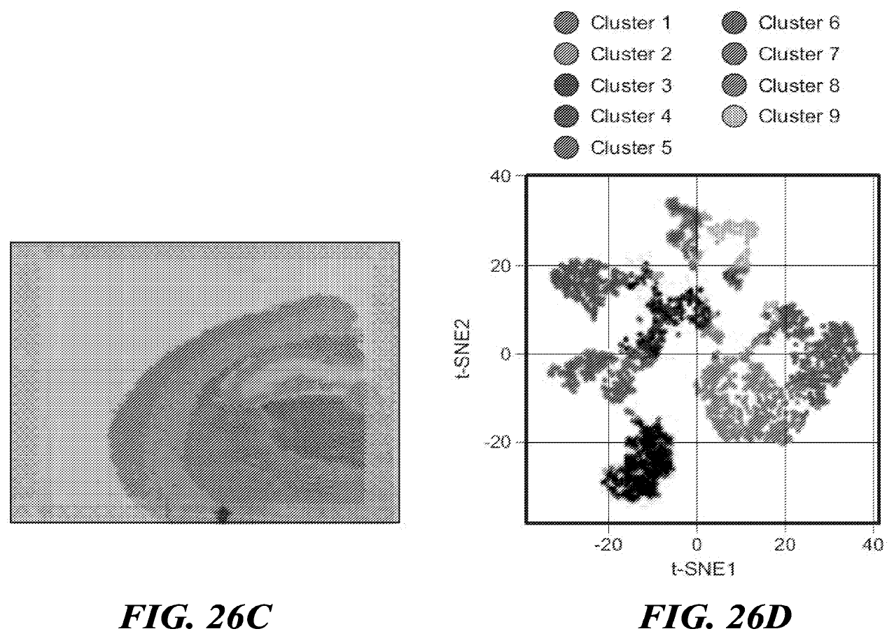

FIGS. 26A-26D show spatial gene expression clustering with a first primer including a poly(T) sequence (e.g., poly(T)30NV) (FIG. 26A) and the corresponding t-SNE plot (FIG. 26B) and spatial gene expression clustering with a first primer including a random decamer (FIG. 26C) and the corresponding t-SNE plot (FIG. 26D) demonstrating that spatial gene expression information can be captured with in situ amplification with a first primer including either a poly(T) sequence or a random decamer sequence and where a complement of a capture sequence is incorporated into extension product(s) described herein.

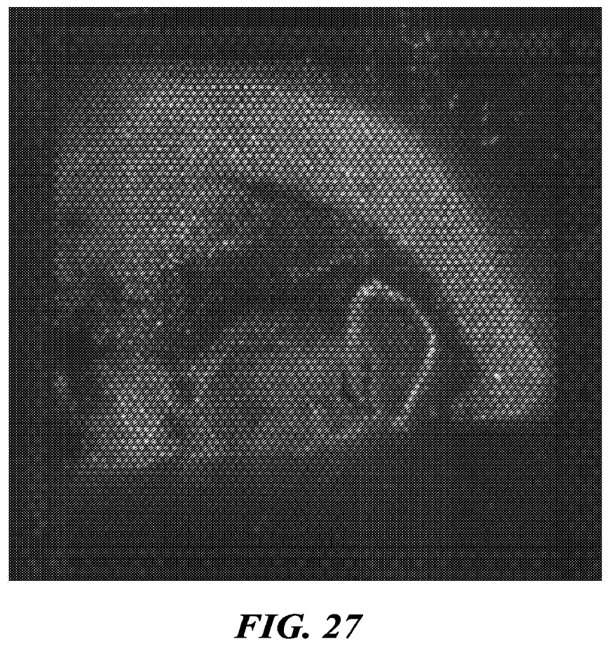

FIG. 27 shows fluorescently labeled extended probes captured in mouse brain tissue using an alternative capture sequence as the handle of the TSO, thereby demonstrating that in situ template switching functions with various sequence handles.

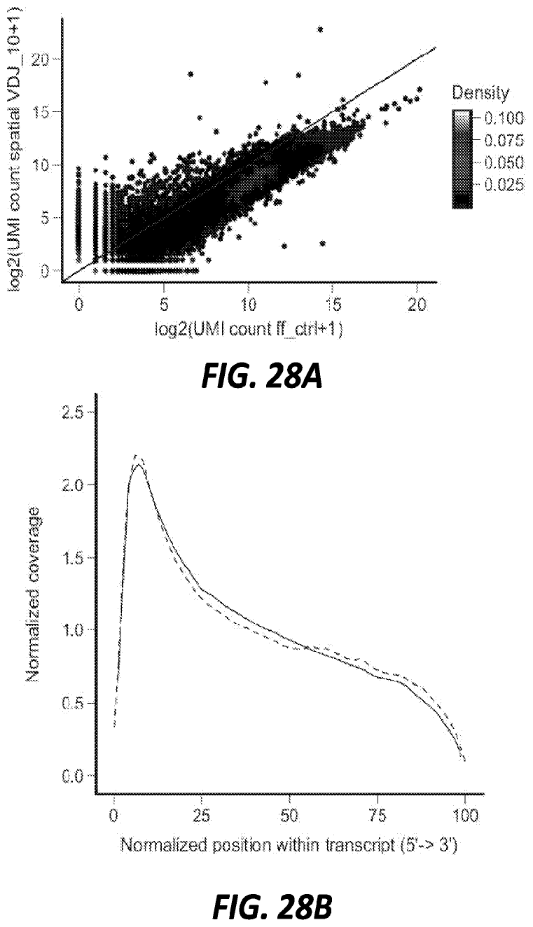

FIGS. 28A-28B are graphs showing correlation between fresh frozen capture using standard Visium spatial gene expression (10× Genomics) and spatial 5′ end capture using the methods disclosed herein (FIG. 28A). Each dot represents the UMI counts for a single gene. FIG. 28B is a graph showing the normalized position of each mapped read within the full-length transcript. The data shown in the graph confirms successful 5′ capture of transcripts.

The methods described herein are also able to identify sequences encoding for a complementarity determining region (“CDR”) e.g., CDR1, CDR2, and/or CDR3 sequences. Determining the sequence of CDRs can also identify clonotypes within a biological sample. Table 1 below shows CDR3 sequences identified from a biological sample. The sequences were obtained from an experiment using fresh frozen Jurkat cell pellets. Jurkat cells express a defined T cell receptor alpha chain (TRA) and T cell receptor beta chain (TRB). The 5′ capture assay was performed with downstream enrichment of VDJ sequences. For this enrichment, primers from single cell 5′GEX Immunoprofiling solution from 10× Genomics were used, followed by library preparation. To analyze the TRA and TRB sequences, the single cell VDJ pipeline (Cell Ranger VDJ) from 10× Genomics was used. The identified TRA and TRB sequences match the expected sequence from the Jurkat reference.

TABLE 1

| Clonotype | | | | | |
|---|---|---|---|---|---|
| ID | SEQ ID NO. | CDR3 | Sequence | Frequency | Proportion |
| 1 | SEQ ID NO: 1 | TRB | CASSFSTCSANYGYTF | 478 | 95.98% |
| | SEQ ID NO: 2 | TRA | CAVSDLEPNSSASKIIF | | |
| 2 | SEQ ID NO: 1 | TRB | CASSFSTCSANYGYTF | 3 | 0.60% |
| 3 | SEQ ID NO: 1 | TRB | CASSFSTCSANYGYTF | 2 | 0.40% |
| 4 | SEQ ID NO: 3 | TRB | CASFSTCSANYGYTF | 1 | 0.20% |
| 5 | SEQ ID NO: 1 | TRB | CASSFSTCSANYGYTF | 1 | 0.20% |
| 6 | SEQ ID NO: 1 | TRB | CASSFSTCSANYGYTF | 1 | 0.20% |
| 7 | SEQ ID NO: 1 | TRB | CASSFSTCSANYGYTF | 1 | 0.20% |
| 8 | SEQ ID NO: 1 | TRB | CASSFSTCSANYGYTF | 1 | 0.20% |
| 9 | SEQ ID NO: 1 | TRB | CASSFSTCSANYGYTF | 1 | 0.20% |
| 10 | SEQ ID NO: 1 | TRB | CASSFSTCSANYGYTF | 1 | 0.20% |

Collectively, the data demonstrate the efficiency of the methods described in Example 7 as a method to enrich target nucleic acids in situ, including target nucleic acids encoding for antibodies and immune cell receptors (e.g., B cell receptors, T cell receptors) and to enrich for 5′ capture of target nucleic acids.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A method for identifying and/or characterizing an antigen-binding molecule (ABM) in a biological sample comprising:
a) contacting the biological sample with a plurality of antigens, wherein the plurality of antigens comprises a target antigen coupled to a first reporter oligonucleotide comprising (i) a first reporter barcode sequence that identifies the target antigen and (ii) a capture handle sequence, and wherein the contacting provides the ABM bound to the target antigen;
b) providing an array of capture probes, the array comprising a first capture probe comprising a spatial first barcode and a first capture domain, and a second capture probe comprising a second spatial barcode and a second capture domain;
c) hybridizing (i) the capture handle sequence of the first reporter oligonucleotide to the first capture domain of the first capture probe and (ii) a nucleic acid comprising a sequence encoding at least a portion of the ABM or a reverse complement thereof to the second capture domain of the second capture probe;
d) generating a first barcoded polynucleotide comprising (i) the first reporter barcode sequence or a reverse complement thereof and (ii) the first spatial barcode or a reverse complement thereof; and
e) generating a second barcoded polynucleotide comprising (i) the nucleic acid encoding at least a portion of the ABM or a reverse complement thereof and (ii) the spatial barcode or a reverse complement thereof.

2. The method of claim 1, further comprising f) determining the identity and spatial location of the ABM in the biological sample and/or identifying the ABM as having bound to the target antigen using the first and second barcoded polynucleotides.

3. The method of claim 1 or 2, wherein the plurality of antigens further comprises a non-target antigen coupled to a second reporter oligonucleotide, wherein the second reporter oligonucleotide comprises (i) a second reporter barcode sequence that identifies the non-target antigen, and (ii) the capture handle sequence.

4. The method of claim 3, further comprising assessing the binding specificity of the ABM; and (i) identifying the ABM as having a binding specificity for the target antigen if the ABM binds to the target antigen and does not significantly bind the non-target antigen, or (ii) identifying the ABM as non-specific for the target antigen if the ABM significantly binds to the non-target antigen.

5. The method of claim 3 or 4, wherein the non-target antigen is an antigen to which the ABM is not expected to bind.

6. The method of any one of claims 1-5, wherein the array comprises a plurality of the first capture probe and a plurality of the second capture probe.

7. The method of any one of claims 1-6, wherein the capture handle sequence of the first reporter oligonucleotide is partially or fully complementary to the first capture domain of the first capture probe.

8. The method of any one of claims 1-7, wherein the first spatial barcode of the first capture probe is identical to or different from the second spatial barcode of the second capture probe, preferably wherein the first spatial barcode is identical to the second spatial barcode.

9. The method of any one of claims 1-8, wherein the plurality of antigens comprises two or more distinct target antigens, wherein each distinct target antigen is coupled to a reporter oligonucleotide comprising (i) a reporter barcode sequence that identifies the target antigen and (ii) the capture handle sequence.

10. The method of any one of claims 1-9, wherein the target antigen and first reporter oligonucleotide are indirectly coupled,
wherein the target antigen is comprised in a labeling agent, wherein the labeling agent further comprises a support, and wherein
(i) the target antigen is coupled to the support via a ligand, and
(ii) the first reporter oligonucleotide is coupled to the support.

11. The method of claim 10, wherein the target antigen is covalently conjugated to the ligand.

12. The method of any one of claims 1-9, wherein the target antigen comprises a target MHC molecule complex, the target MHC molecule complex comprising a MHC molecule bound to a target antigenic molecule,
wherein the target MHC molecule complex is further coupled to a support via a ligand, and wherein the first reporter oligonucleotide is coupled to the support.

13. The method of claim 12, wherein the target antigenic molecule is an antigenic peptide, a lipid, or a small molecule.

14. The method of claim 12 or 13, wherein the target antigen comprises a plurality of target MHC molecule complexes, optionally wherein the target MHC molecule complexes are covalently conjugated to the ligand.

15. The method of any one of claims 12-14, wherein the target antigen comprises four target MHC molecule complexes.

16. The method of any one of claims 10-15, wherein the support comprises a component selected from avidin, streptavidin, deglycosylated avidin (e.g., NeutrAvidin™), traptavidin, tamavidin, xenavidin, bradavidin, AVR2 (avidin related protein 2), AVR4 (avidin related protein 4), and variants, mutants, derivatives, and homologs of any thereof; and/or wherein the ligand comprises biotin.

17. The method of any one of claims 10-16, wherein the labeling agent or target MHC molecule complex further comprises a fluorescent agent.

18. The method of any one of claims 1-17, wherein the ABM is expressed by a cell comprised within the biological sample, optionally wherein the cell is an immune cell.

19. The method of claim 18, wherein the immune cell is a B-cell or T-cell.

20. The method of claim 18 or 19, wherein the ABM is an antibody or fragment thereof, or a T-cell receptor or fragment thereof.

21. The method of claim 20, wherein the antibody is a secreted antibody, optionally wherein the secreted antibody is in proximity to the cell.

22. The method of any one of claims 18-21, wherein step (a) further comprises contacting the biological sample with a plurality of labeling agents, wherein the labeling agents are configured to bind or otherwise couple to one or more surface features of the cell.

23. The method of any one of claims 1-22, wherein the biological sample is disposed (mounted) on the array.

24. The method of any one of claims 1-22, wherein the biological sample is disposed on a first substrate and the array is attached to a second substrate, wherein the method further comprises aligning the first substrate with the second substrate such that at least a portion of the biological sample is aligned with at least a portion of the array.

25. The method of any one of claims 1-22, wherein the biological sample is disposed on a first substrate during (b) and the array is attached to a second substrate, and wherein the method comprises, following (b):
mounting the first substrate on a first member of a support device, the first member configured to retain the first substrate;
mounting the second substrate on a second member of the support device, the second member configured to retain the second substrate,
applying a reagent medium to the first substrate and/or the second substrate, the reagent medium comprising a permeabilization agent,
operating an alignment mechanism of the support device to move the first member and/or the second member such that a portion of the biological sample comprising the ABM is aligned with a portion of the array of capture probes and within a threshold distance of the array of capture probes, and such that the portion of the biological sample and the capture probes contact the reagent medium,
wherein the permeabilization agent releases the nucleic acid comprising a sequence encoding at least a portion of the ABM or a reverse complement thereof from the biological sample.

26. The method of any one of claims 23-25, wherein the method further comprises prior to the hybridizing in step (c), releasing the first reporter oligonucleotide and migrating the first reporter oligonucleotide to the array.

27. The method of claim 25, wherein the nucleic acid comprising a sequence encoding at least a portion of the ABM or a reverse complement thereof is comprised in the biological sample, and the method further comprises releasing the nucleic acid comprising a sequence encoding at least a portion of the ABM or a reverse complement thereof from the biological sample, and migrating the nucleic acid comprising a sequence encoding at least a portion of the ABM or a reverse complement thereof to the array.

28. The method of claim 25 or 26, wherein the migrating comprises passive migration or active migration, optionally wherein the active migration comprises electrophoresis.

29. The method of any one of claims 2-28, wherein the first reporter oligonucleotide and second reporter oligonucleotide further comprise one or more functional domains.

30. The method of any one of claims 1-29, wherein the first and/or second capture probes further comprise a cleavage domain, one or more functional domains, a unique molecular identifier, or a combination thereof.

31. The method of any one of claims 1-30, wherein the first capture domain of the first capture probe and the second capture domain of the second capture probe are identical.

32. The method of any one of claims 1-30, wherein the first capture domain of the first capture probe and the second capture domain of the second capture probe are different.

33. The method of any one of claims 1-32, wherein the first capture domain of the first capture probe is a defined non-homopolymeric sequence or a homopolymeric sequence.

34. The method of claim 33, wherein the homopolymeric sequence comprises a polyT sequence and/or the non-homopolymeric sequence comprises a fixed sequence or a degenerate sequence.

35. The method of any one of claims 1-34, wherein the second capture domain of the second capture probe hybridizes to the nucleic acid comprising a sequence encoding at least a portion of the ABM or a reverse complement thereof, wherein the ABM is an antibody or TCR.

36. The method of claim 35, wherein the second capture domain of the second capture probe hybridizes to a poly(A) sequence of the nucleic acid comprising a sequence encoding at least a portion of the antibody or TCR or a reverse complement thereof.

37. The method of claim 35, wherein the second capture domain of the second capture probe hybridizes to the nucleic acid comprising a sequence encoding at least a portion of the antibody or TCR or the reverse complement thereof in a region encoding a constant region of the antibody or TCR.

38. The method of claim 36 or 37, wherein said generating a second barcoded polynucleotide in step (e) comprises extending the capture probe using the nucleic acid comprising a sequence encoding at least a portion of the antibody or TCR or a reverse complement thereof as a template, thereby generating an extended capture probe; and optionally generating a complement of the extended capture probe.

39. The method of claim 38, further comprising amplifying the second barcoded polynucleotide with a first primer that specifically hybridizes to a functional sequence of the capture probe or reverse complement thereof and a second primer that hybridizes to a nucleic acid sequence encoding a variable region of the antibody or TCR cell or reverse complement thereof, wherein the first primer flanks the spatial barcode of the second barcoded polynucleotide.

40. The method of claim 35, wherein the nucleic acid comprising a sequence encoding at least a portion of the antibody or TCR or a reverse complement thereof comprises a sequence encoding the variable region and constant region of the antibody or TCR.

41. The method of claim 40, wherein said generating a second barcoded polynucleotide in step (e) comprises extending the capture probe using the nucleic acid comprising the sequence encoding the variable region and constant region of the antibody or TCR as a template, thereby generating an extended capture probe; and amplifying the extended capture probe to provide a nucleic acid library.

42. The method of claim 41, comprising circularizing a member of the nucleic acid library to generate a circularized nucleic acid, and amplifying the circularized nucleic acid using a first primer and a second primer to generate a double-stranded member of the nucleic acid library lacking all, or a portion of, the sequence encoding a constant region of the antibody or TCR.

43. The method of any one of claims 1-34, wherein said generating a second barcoded polynucleotide in step (e) comprises
(i) contacting the biological sample with a first primer that hybridizes to the nucleic acid comprising a sequence encoding at least a portion of the ABM or a reverse complement thereof, wherein the first primer comprises a functional domain;
(ii) extending the first primer using the nucleic acid comprising a sequence encoding at least a portion of the ABM or a reverse complement thereof as a template to generate an extension product;
(iii) adding a polynucleotide sequence comprising at least three nucleotides to the 3′ end of the extension product;
(iv) hybridizing a second primer to the polynucleotide sequence comprising at least three nucleotides of the extension product of (iii), wherein the second primer comprises a capture sequence;
(v) extending the extension product using the second primer as a template, thereby incorporating a complement of the capture sequence into the extension product;
(vi) hybridizing the complement of the capture sequence of the extension product to the second capture domain of the second capture probe; and
(vii) extending the 3′ end of the extension product of (v) using the capture probe as a template, thereby generating an extended capture product.

44. The method of claim 43, wherein the first primer hybridizes to a region of the nucleic acid encoding a constant region of the ABM.

45. The method of any one of claims 1-44, wherein said generating a first barcoded polynucleotide in step (d) comprises extending the first reporter oligonucleotide using the first capture probe as a template, thereby providing an extended first reporter oligonucleotide.

46. The method of claim 45, further comprising amplifying the extended first reporter oligonucleotide.

47. The method of any one of claims 3-45 further comprising generating a third barcoded polynucleotide or a plurality of third barcoded polynucleotides comprising (i) the second reporter barcode sequence or reverse complement thereof, and (ii) the first or second spatial barcode or reverse complement thereof, and optionally using the third barcoded polynucleotide or plurality of third barcoded polynucleotides to identify the ABM as having bound to the non-target antigen coupled to the second reporter oligonucleotide.

48. The method of 47, wherein the determining and/or identifying in step (1) comprises determining sequences of the first barcoded polynucleotide and the second barcoded polynucleotide, and optionally determining a sequence of the third barcoded polynucleotide, optionally wherein the determining is performed by sequencing.

49. The method of claim 48 further comprising identifying the ABM based on the determined sequence of the second barcoded polynucleotide.

50. The method of claim 49, wherein the determined sequence comprises a nucleotide sequence.

51. The method of claim 50, wherein the determined sequence comprises an amino acid sequence encoded by the nucleotide sequence.

52. The method of any one of claims 49-51, wherein the binding affinity of the ABM to the target antigen is assessed based on the determined sequence of the first barcoded polynucleotide.

53. The method of any one of claims 1-52, wherein the nucleic acid comprising a sequence encoding at least a portion of the ABM or a reverse complement thereof is RNA or DNA.

54. The method of claim 53, wherein the RNA is mRNA and the DNA is genomic DNA or cDNA.

55. The method of any one of claims 1-54, wherein the method further comprises fixing the biological sample.

56. The method of claim 55, wherein fixing the biological sample comprises the use of a fixative selected from the group consisting of: ethanol, methanol, acetone, formaldehyde, paraformaldehyde-Triton, glutaraldehyde, and combinations thereof.

57. The method of any one of claims 1-56, wherein the method further comprises staining the biological sample.

58. The method of claim 57, wherein the staining comprises use of eosin and/or hematoxylin.

59. The method of claim 57, wherein the staining comprises the use of a detectable label selected from the group consisting of a radioisotope, a fluorophore, a chemiluminescent compound, a bioluminescent compound, or a combination thereof.

60. The method of any one of claims 1-59, wherein the method further comprises imaging the biological sample.

61. The method of claim 60, wherein the imaging comprises one or more of expansion microscopy, bright field microscopy, dark field microscopy, phase contrast microscopy, electron microscopy, fluorescence microscopy, reflection microscopy, interference microscopy and confocal microscopy.

62. The method of any one of claims 1-61, wherein the method further comprises a step of permeabilizing the biological sample, optionally wherein the permeabilizing comprises the use of an organic solvent, a detergent, an enzyme, or a combination thereof.

63. The method of claim 62, wherein the permeabilizing comprises the use of an endopeptidase, wherein the endopeptidase is pepsin or proteinase K, a protease, sodium dodecyl sulfate, polyethylene glycol tert-octylphenyl ether, polysorbate 80, polysorbate 20, N-lauroylsarcosine sodium salt solution, saponin, Triton X-100™, Tween-20™, or combinations thereof.

64. The method of any one of claims 1-63, wherein the biological sample is from a vertebrate subject, optionally wherein the vertebrate subject is a mammalian subject, optionally wherein the mammalian subject is a human.

65. The method of any one of claims 1-63, wherein the biological sample is a tissue sample.

66. The method of claim 65, wherein the tissue sample is a fixed tissue sample, optionally wherein the fixed tissue sample is a formalin-fixed paraffin embedded tissue sample, a paraformaldehyde fixed tissue sample, a methanol fixed tissue sample, or an acetone fixed tissue sample.

67. The method of claim 65, wherein the tissue sample is a fresh frozen tissue sample, optionally a fresh frozen tissue section.

68. The method of any one of claims 1-64, wherein the biological sample is a tissue section.

69. The method of claim 68, wherein the tissue section is a fixed tissue section, optionally wherein the fixed tissue section is a formalin-fixed paraffin embedded tissue section, a paraformaldehyde fixed tissue section, a methanol fixed tissue section, or an acetone fixed tissue section.

70. The method of claim 65, wherein the biological sample is a diseased tissue sample and/or a tissue sample derived from a subject having a disease or disorder.

71. The method of claim 70, wherein the disease or disorder is cancer, an autoimmune disease, a neurodegenerative disease, an infectious disease, or an inflammatory disease.

72. The method of claim 70 or 71, wherein the target antigen is associated with the disease or disorder.

73. The method of claim 72, wherein the target antigen is a cancer antigen or a coronavirus derived antigen.

74. The method of any one of claims 1-73, wherein the target antigen is a peptide.

75. An isolated ABM identified by the method according to any one of claims 1-74.

76. A pharmaceutical composition comprising the isolated ABM of claim 75 or a cell expressing the ABM of claim 75 and a pharmaceutically acceptable excipient.

77. A kit for identifying and/or characterizing an ABM or fragment thereof having binding affinity for an antigen, the kit comprising:
(a) a plurality of target antigens and non-target antigens, wherein each of the target antigens and non-target antigens comprise a reporter oligonucleotide comprising (i) a barcode sequence that identifies the antigen or non-antigen, and (ii) a capture handle sequence;
(b) a spatial array comprising a first capture probe comprising (i) a barcode sequence, and (ii) a first capture domain, and a second capture probe comprising (i) a barcode sequence, and (ii) a second capture domain.

78. The kit of claim 77 further comprising instructions for performing the method of any one of claims 1-74.
